(19) United States
(12) Patent Application Publication
(10) Pub. No.: US 2025/0339034 A1
(43) Pub. Date: Nov. 6, 2025

(54) IMAGING SYSTEM FOR CALCULATING FLUID DYNAMICS

(71) Applicant: Gentuity, LLC, Sudbury, MA (US)

(72) Inventors: Giovanni J. Ughi, Arlington, MA (US); Christopher C. Petroff, Groton, MA (US); Zachary Capalbo, Stoneham, MA (US); Paola Tasso, Somerville, MA (US); Yibo Wu, Windham, NH (US); Taylor Braun-Jones, Brookline, MA (US); R. Maxwell Flaherty, Topsfield, MA (US); J. Christopher Flaherty, Nottingham, NH (US)

(21) Appl. No.: 18/724,841

(22) PCT Filed: Jan. 10, 2023
(86) PCT No.: PCT/US2023/010508

§ 371 Date: Jun. 27, 2024

Related U.S. Application Data
(60) Provisional application No. 63/298,086, filed on Jan. 10, 2022.
(60) Provisional application No. 63/416,170, filed on Oct. 14, 2022.

(51) Int. Cl. A61B 5/026; G06T 7/00; G06T 7/11

(52) U.S. Cl. CPC A61B 5/0084; A61B 5/0066; G06T 7/0012; G06T 7/10; A61B 5/026; G06T 2200/24; G06T 2207/10101; G06T 2207/20081; G06T 2207/20084; G06T 2207/30104

(57) ABSTRACT

Provided herein are imaging systems for a patient including an imaging probe and an imaging assembly. The imaging probe includes an elongate shaft with a rotatable optical core positioned within a lumen of the elongate shaft. The imaging probe further includes an optical assembly to direct light to tissue to be imaged and to collect reflected light from the tissue to be imaged. The system further includes an imaging assembly optically coupled to the imaging probe. The system further includes a processing unit with a processor and a memory coupled to the processor, and the memory stores instructions for the processor to perform an algorithm. The system records image data based on the reflected light collected by the optical assembly, such that the image data comprises data collected from a segment of a blood vessel during a pullback procedure. The algorithm can analyze the image data.

### RELATED APPLICATIONS

This application claims benefit of U.S. Provisional Application Ser. No. 63/298,086 (Docket No. GTY-022-PR1), titled “Imaging System for Calculating Fluid Dynamics”, filed Jan. 10, 2022, the content of which is incorporated by reference in its entirety.

This application claims benefit of U.S. Provisional Application Ser. No. 63/416,170 (Docket No. GTY-023-PR1), titled “Imaging System”, filed Oct. 14, 2022, the content of which is incorporated by reference in its entirety.

This application is related to U.S. Provisional Application Ser. No. 62/148,355 (Docket No.: GTY-001-PR1), titled “Micro-Optic Probes for Neurology”, filed Apr. 16, 2015, the content of which is incorporated by reference in its entirety.

This application is related to U.S. Provisional Application Ser. No. 62/322,182 (Docket No. GTY-001-PR2), titled “Micro-Optic Probes for Neurology”, filed Apr. 13, 2016, the content of which is incorporated by reference in its entirety.

This application is related to International PCT Patent Application Serial Number PCT/US2016/027764 (Docket No. GTY-001-PCT), titled “Micro-Optic Probes for Neurology” filed Apr. 15, 2016, Publication Number WO 2016/168605, published Oct. 20, 2016, the content of which is incorporated by reference in its entirety.

This application is related to U.S. patent application Ser. No. 15/566,041 (Docket No. GTY-001-US), titled “Micro-Optic Probes for Neurology”, filed Oct. 12, 2017, U.S. Pat. No. 11,278,206, issued Mar. 22, 2022, the content of which is incorporated by reference in its entirety.

This application is related to U.S. patent application Ser. No. 17/668,757 (Docket No. GTY-001-US-CON1), titled “Micro Optic Probes for Neurology”, filed Feb. 10, 2022, United States Publication Number 2022-0218206, published Jul. 14, 2022, the content of which is incorporated by reference in its entirety.

This application is related to U.S. Provisional Application Ser. No. 62/212,173 (Docket No. GTY-002-PR1), titled “Imaging System Includes Imaging Probe and Delivery Devices”, filed Aug. 31, 2015, the content of which is incorporated by reference in its entirety.

This application is related to U.S. Provisional Application Ser. No. 62/368,387 (Docket No. GTY-002-PR2), titled “Imaging System Includes Imaging Probe and Delivery Devices”, filed Jul. 29, 2016, the content of which is incorporated by reference in its entirety.

This application is related to International PCT Patent Application Serial Number PCT/US2016/049415 (Docket No. GTY-002-PCT), titled “Imaging System Includes Imaging Probe and Delivery Devices”, filed Aug. 30, 2016, Publication Number WO 2017/040484, published Mar. 9, 2017, the content of which is incorporated by reference in its entirety.

This application is related to U.S. patent application Ser. No. 15/751,570 (Docket No. GTY-002-US), titled “Imaging System Includes Imaging Probe and Delivery Devices”, filed Feb. 9, 2018, U.S. Pat. No. 10,631,718, issued Apr. 28, 2020, the content of which is incorporated by reference in its entirety.

This application is related to U.S. patent application Ser. No. 16/820,991 (Docket No. GTY-002-US-CON1), titled “Imaging System Includes Imaging Probe and Delivery Devices”, filed Mar. 17, 2020, U.S. Pat. No. 11,064,873, issued Jul. 20, 2021, the content of which is incorporated by reference in its entirety.

This application is related to U.S. patent application Ser. No. 17/350,021 (Docket No. GTY-002-US-CON2), titled “Imaging System Includes Imaging Probe and Delivery Devices”, filed Jun. 17, 2021, Publication Number 2022-0142464, published May 12, 2022, the content of which is incorporated by reference in its entirety.

This application is related to U.S. Provisional Application Ser. No. 62/591,403 (Docket No. GTY-003-PR1), titled “Imaging System”, filed Nov. 28, 2017, the content of which is incorporated by reference in its entirety.

This application is related to U.S. Provisional Application Ser. No. 62/671,142 (Docket No. GTY-003-PR2), titled “Imaging System”, filed May 14, 2018, the content of which is incorporated by reference in its entirety.

This application is related to International PCT Patent Application Serial Number PCT/US2018/062766 (Docket No. GTY-003-PCT), titled “Imaging System”, filed Nov. 28, 2018, Publication Number WO 2019/108598, published Jun. 6, 2019, the content of which is incorporated by reference in its entirety.

This application is related to U.S. patent application Ser. No. 16/764,087 (Docket No. GTY-003-US), titled “Imaging System”, filed May 14, 2020, Publication Number 2020-0288950, published Sep. 17, 2020, the content of which is incorporated by reference in its entirety.

This application is related to U.S. Provisional Application Ser. No. 62/732,114 (Docket No. GTY-004-PR1), titled “Imaging System with Optical Pathway”, filed Sep. 17, 2018, the content of which is incorporated by reference in its entirety.

This application is related to International PCT Patent Application Serial Number PCT/US2019/051447 (Docket No. GTY-004-PCT), titled “Imaging System with Optical Pathway”, filed Sep. 17, 2019, Publication Number WO 2020/061001, published Mar. 26, 2020, the content of which is incorporated by reference in its entirety.

This application is related to U.S. patent application Ser. No. 17/276,500 (Docket No. GTY-004-US), filed Mar. 16, 2021, titled “Imaging system with Optical Pathway”, Publication Number 2021-0267442, published Sep. 2, 2021, the content of which is incorporated by reference in its entirety.

This application is related to U.S. Provisional Application Ser. No. 63/017,258 (Docket No. GTY-005-PR1), titled “Imaging System”, filed Apr. 29, 2020, the content of which is incorporated by reference in its entirety.

This application is related to International PCT Patent Application Serial Number PCT/US2021/29836 (Docket No. GTY-005-PCT), titled “Imaging System”, filed Apr. 29, 2021, Publication Number WO 2021/222530, published Nov. 4, 2021, the content of which is incorporated by reference in its entirety.

This application is related to U.S. patent application Ser. No. 17/919,809 (Docket No. GTY-005-US), filed Oct. 19, 2022, titled “Imaging System”, Publication Number ______, published ______, the content of which is incorporated by reference in its entirety.

This application is related to U.S. Provisional Application Ser. No. 62/840,450 (Docket No. GTY-011-PR1), titled “Imaging Probe with Fluid Pressurization Element”, filed Apr. 30, 2019, the content of which is incorporated by reference in its entirety.

This application is related to International PCT Patent Application Serial Number PCT/US2020/030616 (Docket No. GTY-011-PCT), titled “Imaging Probe with Fluid Pressurization Element”, filed Apr. 30, 2020, Publication Number WO 2020/223433, published Nov. 5, 2020, the content of which is incorporated by reference in its entirety.

This application is related to U.S. patent application Ser. No. 17/600,212 (Docket No. GTY-011-US), titled “Imaging Probe with Fluid Pressurization Element”, filed Sep. 30, 2021, Publication Number 2022-0142462, published May 12, 2022, the content of which is incorporated by reference in its entirety.

This application is related to U.S. Provisional Application Ser. No. 62/850,945 (Docket No. GTY-013-PR1), titled “OCT-Guided Treatment of a Patient”, filed May 21, 2019, the content of which is incorporated by reference in its entirety.

This application is related to U.S. Provisional Application Ser. No. 62/906,353 (GTY-013-PR2), titled “OCT-Guided Treatment of a Patient”, filed Sep. 26, 2019, the content of which is incorporated by reference in its entirety.

This application is related to International PCT Patent Application Serial Number PCT/US2020/033953 (Docket No. GTY-013-PCT), titled “Systems and Methods for OCT-Guided Treatment of a Patient”, filed May 21, 2020, Publication Number WO 2020/237024, published Nov. 26, 2020, the content of which is incorporated by reference in its entirety.

This application is related to U.S. patent application Ser. No. 17/603,689 (Docket No. GTY-013-US), titled “Systems and Methods for OCT-Guided Treatment of a Patient”, filed Oct. 14, 2021, Publication Number 2022-0061670, published Mar. 3, 2022, the content of which is incorporated by reference in its entirety.

This application is related to U.S. Provisional Application Ser. No. 63/154,934 (Docket No. GTY-021-PR1), titled “Optical Imaging System”, filed Mar. 1, 2021, the content of which is incorporated by reference in its entirety.

This application is related to U.S. patent application Ser. No. 17/682,197 (Docket No. GTY-021-US), titled “Optical Imaging System”, filed Feb. 28, 2022, Publication Number 2023-0000321, published Jan. 5, 2023, the content of which is incorporated by reference in its entirety.

### FIELD OF THE INVENTIVE CONCEPTS

The present invention relates generally to imaging systems, and in particular, intravascular imaging systems including imaging probes and delivery devices.

### BACKGROUND

Imaging probes have been commercialized for imaging various internal locations of a patient, such as an intravascular probe for imaging a patient's heart. Current imaging probes are limited in their ability to reach certain anatomical locations due to their size and rigidity. Current imaging probes are inserted over a guidewire, which can compromise their placement and limit use of one or more delivery catheters through which the imaging probe is inserted. There is a need for imaging systems that include probes with reduced diameter and high flexibility, as well as systems with one or more delivery devices compatible with these improved imaging probes.

### SUMMARY

According to an aspect of the present inventive concepts, an imaging system for a patient comprises an imaging probe comprising an elongate shaft comprising a proximal end, a distal portion, and a lumen extending between the proximal end and the distal portion. The imaging probe further comprises a rotatable optical core comprising a proximal end and a distal end, and at least a portion of the rotatable optical core is positioned within the lumen of the elongate shaft. The imaging probe further comprises an optical assembly positioned proximate the distal end of the rotatable optical core, and the optical assembly is configured to direct light to tissue to be imaged and to collect reflected light from the tissue to be imaged. The system further comprises an imaging assembly constructed and arranged to optically couple to the imaging probe, and the imaging assembly is configured to emit light into the imaging probe and to receive the reflected light collected by the optical assembly. The system further comprises a processing unit comprising a processor and a memory coupled to the processor, and the memory is configured to store instructions for the processor to perform an algorithm. The system can be configured to record image data based on the reflected light collected by the optical assembly, such that the image data comprises data collected from a segment of a blood vessel during a pullback procedure. The algorithm can be configured to analyze the image data.

In some embodiments, the image data comprises OCT image data.

In some embodiments, the algorithm is configured to calculate computational fluid dynamics of the vessel segment.

In some embodiments, the algorithm is configured to segment the image data. The segmentation can be selected from the group consisting of: procedural device segmentation; guide catheter segmentation; guidewire segmentation; implant segmentation; endovascular implant segmentation; flow-diverter segmentation; lumen segmentation; side-branch segmentation, and combinations thereof. The algorithm can comprise a neural network tailored to perform the segmentation.

In some embodiments, the algorithm is configured to produce a confidence metric configured to represent the quality of the results of an image processing step.

In some embodiments, the algorithm comprises an artificial intelligence algorithm. The artificial intelligence algorithm can comprise a machine learning algorithm, a deep learning algorithm, or a neural network. The algorithm can comprise a neural network and can be configured to skip one or more layers of the neural network. The algorithm can comprise a single neural network trained to perform two or more image segmentation processes. The artificial intelligence algorithm can be trained to perform a side-branch segmentation, and the algorithm achieves an average Weighted Dice Score of at least 0.81.

In some embodiments, the algorithm is configured to receive image data in a single image domain, and the algorithm is further configured to convert the image data into one or more additional image domains.

In some embodiments, the algorithm is configured to process the image data in one or more image domains selected from the group consisting of: the polar domain; the cartesian domain; the longitudinal domain; the en-face image domain; a domain generated by calculating image features, such as first and/or second order features, image texture, image entropy, homogeneity, correlation, contrast, energy, and/or any other image feature; and combinations thereof.

In some embodiments, the system further comprises a graphical user interface configured to be displayed to a user. The graphical user interface can be configured to provide an image data quality indicator. The image data quality indicator can be displayed relative to a cross-sectional OCT image. The graphical user interface can be configured to enable a user to review the results of an image processing step. The graphical user interface can be further configured to enable a user to approve the results of the image processing step. The graphical user interface can be further configured to enable a user to edit the results of the image processing step. The algorithm can comprise an artificial intelligence algorithm, and the image processing step can be performed by the artificial intelligence algorithm. The graphical user interface can comprise multiple workspaces, and the data displayed in each workspace can be synchronized. The data can be synchronized by a time index. The data can be synchronized by a location index.

In some embodiments, the system is configured to collect image data prior to an interventional procedure and after the interventional procedure. The algorithm can be configured to compare the pre-intervention image data and the post-intervention image data and to quantify the effect of the interventional procedure. The algorithm can comprise an artificial intelligence algorithm.

In some embodiments, the algorithm comprises a bias. The system can comprise a user interface, and the bias can be entered and/or modified via the user interface.

The technology described herein, along with the attributes and attendant advantages thereof, will best be appreciated and understood in view of the following detailed description taken in conjunction with the accompanying drawings in which representative embodiments are described by way of example.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic view of a diagnostic system comprising an imaging probe and one or more algorithms for processing image data, consistent with the present inventive concepts.

FIG. 2 illustrates a graphical representation of a neural network, consistent with the present inventive concepts.

FIG. 3 illustrates an embodiment of a graphical user interface for displaying image data and guiding vascular intervention, consistent with the present inventive concepts.

FIGS. 3A-3C illustrate additional embodiments of a graphical user interface for displaying image data and guiding vascular intervention, consistent with the present inventive concepts.

FIGS. 4A-4D illustrate anatomic views of a vessel showing various levels of atherosclerosis, consistent with the present inventive concepts.

FIG. 5 illustrates a method of procedure planning based on data collected and/or analyzed by the system, consistent with the present inventive concepts.

FIGS. 6A-C illustrate various OCT images of vessels and guide catheters, consistent with the present inventive concepts.

FIGS. 7A-7D illustrate images to be displayed to a user representing OCT image data and image quality, consistent with the present inventive concepts.

FIGS. 8 and 8A illustrate embodiments of a graphical user interface for displaying image data and allowing a user to review information determined by the system based off of the image data, consistent with the present inventive concepts.

FIGS. 9-12B illustrate various representations of data collected by the applicant, consistent with the present inventive concepts.

FIG. 13 illustrates OCT image data showing the results of poor catheter purging and good catheter purging, consistent with the present inventive concepts.

FIG. 14 illustrates another embodiment of a graphical user interface for displaying image data and guiding vascular intervention, consistent with the present inventive concepts.

FIG. 15 illustrates a method of treating a patient including planning and evaluating a treatment plan, consistent with the present inventive concepts.

FIGS. 16A-E illustrate examples of various types of image data, consistent with the present inventive concepts.

FIG. 17 illustrates an embodiment of a graphical user interface for displaying image features automatically identified by an image processing algorithm, consistent with the present inventive concepts.

FIGS. 18A-18C illustrate preprocessed examples of image data with varying levels of blood in each image, consistent with the present inventive concepts.

FIGS. 19A-C illustrate additional OCT images, consistent with the present inventive concepts.

FIG. 20 illustrates results of testing performed by the applicant, consistent with the present inventive concepts.

FIG. 21 illustrates a graphical representation of a neural network, consistent with the present inventive concepts.

FIGS. 21A and 21B illustrate an image frame and longitudinal image data, respectively, consistent with the present inventive concepts.

FIGS. 22A and 22B illustrate a representation of the combined method segmentation and an example of segmented image data, respectively, consistent with the present inventive concepts.

FIGS. 23 and 24 illustrate various representations of data collected by the applicant, consistent with the present inventive concepts.

FIGS. 25A-26B illustrate various representations of data collected by the applicant, consistent with the present inventive concepts.

FIGS. 27A-28 illustrate various representations of data collected by the applicant, consistent with the present inventive concepts.

FIG. 29 illustrates a method of capturing image data, applying AI algorithms on the data to develop improved medical procedures, and obtaining regulatory authority clearance of these procedures, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

As used herein, the terms “operably attached”, “operably connected”, “operatively coupled” and similar terms related to attachment of components shall refer to attachment of two or more components that results in one, two, or more of: electrical attachment; fluid attachment; magnetic attachment; mechanical attachment; optical attachment; sonic attachment; and/or other operable attachment arrangements. The operable attachment of two or more components can facilitate the transmission between the two or more components of: power; signals; electrical energy; fluids or other flowable materials; magnetism; mechanical linkages; light; sound such as ultrasound; and/or other materials and/or components.

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the terms “about” or “approximately” shall refer to ±5% of a stated value.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” where used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross sectional area as the cross section of the component being described.

The terms “major axis” and “minor axis” of a component where used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” where used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems for diagnosing and/or treating a patient, such as to be used in a medical procedure comprising a diagnostic procedure, a therapeutic procedure (also referred to as a “treatment procedure”), or both. The systems of the present inventive concepts comprise an imaging probe and an imaging assembly. The imaging probe can comprise an elongate shaft, a rotatable optical core, and an optical assembly. The shaft can comprise a proximal end, a distal portion, and a lumen extending between the proximal end and the distal portion. The rotatable optical core can comprise a proximal end and a distal end, and at least a portion of the rotatable optical core can be positioned within the lumen of the elongate shaft. The optical assembly can be positioned proximate the distal end of the rotatable optical core, and can be configured to direct light to tissue and collect reflected light from the tissue. The imaging systems can comprise one or more algorithms configured to enhance the performance of the system.

The imaging systems of the present inventive concepts can be used to provide image data representing arteries, veins, and/or other body conduits, and to image one or more devices inserted into those conduits. The imaging system can be used to image tissue and/or other structures outside of the blood vessel and/or other lumen into which the imaging probe is inserted. The imaging systems can provide image data related to healthy tissue, as well as diseased tissue, such as blood vessels including a stenosis, myocardial bridge, and/or other vessel narrowing (“lesion” or “stenosis” herein), and/or blood vessels including an aneurysm. The systems can be configured to provide treatment information (e.g. suggested treatment steps to be performed), such as when the treatment information is used by an operator (e.g. a clinician of the patient) to plan a treatment and/or to predict a treatment outcome.

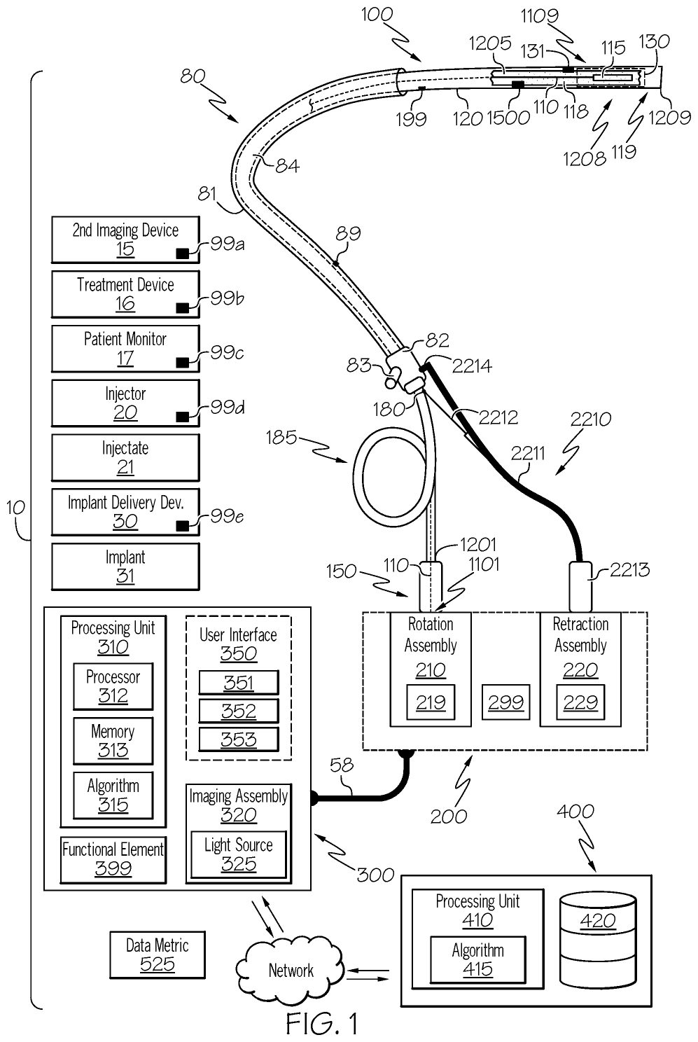

Referring now to FIG. 1, a schematic view of a diagnostic system comprising an imaging probe and one or more algorithms for processing image data is illustrated, consistent with the present inventive concepts. System 10 can be configured as a diagnostic system that is configured to record image data from a patient and produce one or more images based on the recorded data. System 10 can be further configured to analyze the recorded data and/or the produced images (either or both, “image data” herein), such as to provide: diagnostic data relating to a disease or condition of a patient; planning data relating to the planning of a treatment procedure to be performed on a patient; and/or outcome data relating to the efficacy and/or technical outcomes of a treatment procedure. Diagnostic data can include image data.

System 10 can be constructed and arranged to record optical coherence tomography (OCT) data from an imaging location (e.g. OCT data recorded from a segment of a blood vessel during a pullback procedure, as described herein). In some embodiments, the OCT data recorded by system 10 comprises high-frequency OCT (HF-OCT) data. System 10 can comprise a catheter-based probe, imaging probe 100, as well as a probe interface unit, PIU 200, that is configured to operably attach to imaging probe 100. PIU 200 can comprise rotation assembly 210 and/or retraction assembly 220, where each of these assemblies can operably attach to imaging probe 100 to rotate and/or retract, respectively, at least a portion of imaging probe 100. System 10 can comprise console 300 that operably attaches to imaging probe 100, such as via PIU 200. Imaging probe 100 can be introduced into a conduit of the patient, such as a blood vessel or other conduit of the patient, using (e.g. passing through) one or more delivery catheters, delivery catheter 80 shown. Additionally or alternatively, imaging probe 100 can be introduced through an introducer device, such as an endoscope, arthroscope, balloon dilator, or the like. In some embodiments, imaging probe 100 is configured to be introduced into a patient conduit and/or other patient internal site selected from the group consisting of: an artery; a vein; an artery within or proximate the heart; a vein within or proximate the heart; an artery within or proximate the brain; a vein within or proximate the brain; a peripheral artery; a peripheral vein; a patient internal site that is accessed through a natural body orifice, such as the esophagus; a patient internal site that is accessed through a surgically created orifice, such as a conduit or other site within the abdomen; and combinations of one or more of these.

In some embodiments, imaging probe 100 and/or another component of system 10 can be of similar construction and arrangement to the similar components described in applicant's co-pending U.S. patent application Ser. No. 17/668,757 (Docket No. GTY-001-US-CON1), titled “Micro-Optic Probes for Neurology”, filed Feb. 10, 2022. Imaging probe 100 can be constructed and arranged to collect image data from a patient site, such as an intravascular cardiac site, an intracranial site, or other site accessible via the vasculature of the patient. In some embodiments, system 10 can be of similar construction and arrangement to the similar systems and their methods of use described in applicant's co-pending U.S. patent application Ser. No. 17/350,021 (Docket No. GTY-002-US-CON2), titled “Imaging System Includes Imaging Probe and Delivery Devices”, filed Jun. 17, 2021.

Imaging probe 100 can comprise an elongate body comprising one or more elongate shafts and/or tubes, shaft 120 herein. Shaft 120 comprises a proximal end 1201, distal end 1209, and a lumen 1205 extending therebetween. In some embodiments, lumen 1205 can include multiple coaxial lumens within the one or more elongate shafts of shaft 120, such as one or more lumens (e.g. axially aligned lumens) abutting each other to define a single lumen 1205. In some embodiments, at least a portion of shaft 120 comprises a torque shaft. In some embodiments, a portion of shaft 120 comprises a braided construction. In some embodiments, a portion of shaft 120 comprises a spiral cut tube (e.g. shaft 120 includes a spiral cut metal tube). In some embodiments, the pitch of the spiral cut can be varied along the length of the cut, such as to vary the stiffness of shaft 120 along its length. A portion of shaft 120 can comprise a tube constructed of nickel-titanium alloy. Shaft 120 operably surrounds a rotatable optical fiber, optical core 110 (e.g. optical core 110 is positioned within lumen 1205), where core 110 comprises a proximal end 1101 and a distal end 1109. Optical core 110 can comprise a dispersion shifted optical fiber, such as a depressed cladding dispersion shifted fiber (e.g. a Non-Zero Dispersion Shifted, NZDS, fiber). Shaft 120 further comprises a distal portion 1208, including a transparent portion, window 130 (e.g. a window that is relatively transparent to the one or more frequencies of light transmitted through optical core 110). An optical assembly, optical assembly 115, is operably attached to the distal end 1109 of optical core 110. Optical assembly 115 is positioned within window 130 of shaft 120. Optical assembly 115 can comprise a GRIN lens optically coupled to the distal end 1109 of optical core 110. Optical assembly 115 can comprise a construction and arrangement similar to optical assembly 115 as described in applicant's co-pending U.S. patent application Ser. No. 16/764,087 (Docket No. GTY-003-US), titled “Imaging System”, filed May 14, 2020, and applicant's co-pending U.S. patent application Ser. No. 17/276,500 (Docket No. GTY-004-US), titled “Imaging System with Optical Pathway”, filed Mar. 16, 2021. In some embodiments, optical core 110 comprises a single continuous length of optical fiber comprising zero splices along its length. In some embodiments, imaging probe 100 comprises a single optical splice, such as a splice being between optical assembly 115 and distal end 1109 of optical core 110 (e.g. when there are zero splices along the length of optical core 110).

A connector assembly, connector assembly 150, is positioned on the proximal end of shaft 120. Connector assembly 150 operably attaches imaging probe 100 to rotation assembly 210. In some embodiments, connector assembly 150 comprises an optical connector fixedly attached to the proximal end of optical core 110. Imaging probe 100 can comprise a second connector, connector 180, that can be positioned on shaft 120. Connector 180 can be removably attached and/or adjustably positioned along the length of shaft 120. Connector 180 can be positioned along shaft 120, such as by a clinician, technician, and/or other user of system 10 (“user” or “operator” herein), proximate the proximal end of delivery catheter 80 after imaging probe 100 has been inserted into a patient via delivery catheter 80. Shaft 120 can comprise a portion between connector assembly 150 and the placement location of connector 180 that is configured to provide and/or accommodate slack in shaft 120, service loop 185.

In some embodiments, shaft 120 comprises a multi-part construction, such as an assembly of two or more tubes that can be connected in various ways. In some embodiments, one or more tubes of shaft 120 can comprise tubes made of polyethylene terephthalate (PET), such as when a PET tube surrounds the junction between two tubes (e.g. two portions of shaft 120) in an axial arrangement to create a joint between the two tubes. In some embodiments, one or more PET tubes are under tension after assembly (e.g. the tubes are longitudinally stretched when shaft 120 is assembled), such as to prevent or at least reduce the tendency of the PET tube to wrinkle while shaft 120 is advanced through a tortuous path. In some embodiments, one or more portions of shaft 120 include a coating comprising one, two, or more materials and/or surface modifying processes, such as to provide a hydrophilic coating or a lubricious coating. In some embodiments, one or more metal portions of shaft 120 (e.g. nickel-titanium portions) are surrounded by a tube (e.g. a polymer tube), such as to improve the adhesion of a coating to that portion of shaft 120.

Imaging probe 100 can comprise one or more visualizable markers along its length (e.g. along shaft 120), marker 131 shown. Marker 131 can comprise one or more markers selected from the group consisting of: radiopaque markers; ultrasonically reflective markers; magnetic markers; ferrous material; and combinations of one or more of these. In some embodiments, marker 131 is positioned at a location along imaging probe 100 selected to assist an operator of system 10 in performing a pullback procedure (“pullback procedure” or “pullback” herein). For example, marker 131 can be positioned approximately one pullback length from distal end 1209 of shaft 120, such that following a pullback, distal end 1209 will be no more proximal than the starting position of marker 131. In some embodiments, prior to a pullback, the operator can position marker 131 at a location distal to the proximal end of an implant, such that after the pullback is completed access into the implant is maintained (e.g. such that imaging probe 100 can be safely advanced through the implant after the pullback).

In some embodiments, imaging probe 100 includes a viscous dampening material, gel 118, positioned within shaft 120 and surrounding optical assembly 115 and a distal portion of optical core 110 (e.g. a gel injected or otherwise installed in a manufacturing process). Gel 118 can comprise a non-Newtonian fluid, for example a shear-thinning fluid. In some embodiments, gel 118 comprises a static viscosity of at least 500 centipoise, and a shear viscosity that is less than the static viscosity. In these embodiments, the ratio of static viscosity to shear viscosity of gel 118 can be between 1.2:1 and 100:1. In some embodiments, gel 118 is injected from the distal end of window 130 (e.g. in a manufacturing process). In some embodiments, gel 118 comprises a gel which is visualizable (e.g. visualizable under UV light, such as when gel 118 includes one or more materials that fluoresce under UV light). In some embodiments, during a manufacturing process in which gel 118 is injected into shaft 120 via window 130, shaft 120 is monitored while gel 118 is visualized (e.g. being illuminated by UV light) such that the injection process can be controlled (e.g. injection is stopped when gel 118 sufficiently ingresses into shaft 120). Gel 118 can comprise a gel as described in reference to applicant's co-pending U.S. patent application Ser. No. 17/668,757 (Docket No. GTY-001-US-CON1), titled “Micro-Optic Probes for Neurology”, filed Oct. 12, 2017, and applicant's co-pending U.S. patent application Ser. No. 16/764,087 (Docket No. GTY-003-US), titled “Imaging System”, filed May 14, 2020.

Imaging probe 100 can include a distal tip portion, distal tip 119. In some embodiments, distal tip 119 can comprise a spring tip, such as a spring tip configured to improve the “navigability” of imaging probe 100 (e.g. to improve “trackability” and/or “steerability” of imaging probe 100), for example when probe 100 is translated within a tortuous pathway (e.g. within a blood vessel of the brain or heart with a tortuous pathway). In some embodiments, distal tip 119 comprises a length of between 5 mm and 100 mm (e.g. a spring with a length between 5 mm and 100 mm). In some embodiments, distal tip 119 can comprise a user shapeable spring tip (e.g. at least a portion of distal tip 119 is malleable). Imaging probe 100 can be rotated (e.g. via connector 180) to adjust the direction of a non-linear shaped portion of distal tip 119 (e.g. to adjust the trajectory of distal tip 119 in the vasculature of the patient). Alternatively or additionally, distal tip 119 can comprise a cap, plug, and/or other element configured to seal the distal opening of window 130. In some embodiments, distal tip 119 can comprise a radiopaque marker configured to increase the visibility of imaging probe 100 under a fluoroscope or other X-ray device. In some embodiments, distal tip 119 can comprise a relatively short luminal guidewire pathway to allow “rapid exchange” translation of imaging probe 100 over a guidewire of system 10 (guidewire not shown).

In some embodiments, at least the distal portion of imaging probe 100 (e.g. the distal portion of shaft 120 surrounding optical assembly 115) comprises an outer diameter of no more than 0.030″, such as no more than 0.025″, no more than 0.020″, and/or no more than 0.016″.

In some embodiments, imaging probe 100 can be constructed and arranged for use in an intravascular neural procedure (e.g. a procedure in which the blood, vasculature, and other tissue proximate the brain are visualized, and/or devices positioned temporarily or permanently proximate the brain are visualized). An imaging probe 100 configured for use in an intravascular neural procedure (also referred to herein as a “neural procedure”) can comprise an overall length of at least 150 cm, such as a length of approximately 300 cm. Alternatively or additionally, imaging probe 100 can be constructed and arranged for use in an intravascular cardiac procedure (e.g. a procedure in which the blood, vasculature, and other tissue proximate the heart are visualized, and/or devices positioned temporarily or permanently proximate the heart are visualized). An imaging probe 100 configured for use in an intravascular cardiac procedure (e.g. also referred to as a “cardiac procedure” or “cardiovascular procedure” herein) can comprise an overall length of at least 120 cm, such as an overall length of approximately 280 cm (e.g. to allow placement of the proximal end of imaging probe 100 outside of the sterile field). In some embodiments, such as for placement of the proximal end of probe 100 outside of the sterile field, imaging probe 100 can comprise a length greater than 220 cm, such as a length of at least 220 cm but less than 320 cm.

In some embodiments, imaging probe 100 comprises an element, FPE 1500 shown, which can be configured as a fluid propulsion element and/or a fluid pressurization element (“fluid pressurization element” herein). FPE 1500 can be configured to prevent and/or reduce the presence of bubbles within gel 118 proximate optical assembly 115. FPE 1500 can be fixedly attached to optical core 110, wherein rotation of optical core 110 in turn rotates FPE 1500, such as to generate a pressure increase within gel 118 that is configured to reduce presences of bubbles from locations proximate optical assembly 115. Such one or more fluid pressurization elements FPE 1500 can be constructed and arranged to: reduce the likelihood of bubble formation within gel 118, reduce the size of bubbles within gel 118, and/or move any bubbles formed within gel 118 away from a location that would adversely impact the collecting of image data by optical assembly 115 (e.g. move bubbles away from optical assembly 115). In some embodiments, a fluid propulsion element FPE 1500 of imaging probe 100 comprises a similar construction and arrangement to a fluid propulsion element described in applicant's co-pending U.S. patent application Ser. No. 17/600,212 (Docket No. GTY-011-US), titled “Imaging Probe with Fluid Pressurization Element”, filed Sep. 30, 2021.

In some embodiments, delivery catheter 80 comprises an elongate shaft, shaft 81 shown, which includes a lumen 84 therethrough and a connector 82 positioned on its proximal end. Connector 82 can comprise a Touhy or other valved connector, such as a valved connector configured to prevent fluid egress from the associated delivery catheter 80 (with and/or without a separate shaft positioned within the connector 82). Connector 82 can comprise port 83, such as one or more ports constructed and arranged to allow introduction of fluid into delivery catheter 80 and/or for removing fluids from delivery catheter 80. In some embodiments, a flushing fluid, such as is described herein, is introduced via one or more ports 83, such as to remove blood or other undesired material from locations proximate optical assembly 115 (e.g. from a location proximal to optical assembly 115 to a location distal to optical assembly 115). Port 83 can be positioned on a side of connector 82 and can include a luer fitting and a cap and/or valve. Shafts 81, connectors 82, and ports 83 can each comprise standard materials and be of similar construction to commercially available introducers, guide catheters, diagnostic catheters, intermediate catheters and microcatheters used in interventional procedures today. Delivery catheter 80 can comprise a catheter configured to deliver imaging probe 100 to an intracerebral location, an intracardiac location, and/or another location within a patient.

Delivery catheter 80 can comprise two or more delivery catheters, such as three or more delivery catheters. Delivery catheter 80 can comprise at least a vascular introducer, and other delivery catheters that can be inserted into the patient (e.g. through the vascular introducer, after the vascular introducer is positioned through the skin of the patient). Delivery catheter 80 can comprise sets of two or more delivery catheters collectively comprising sets of various inner diameters (IDs) and outer diameters (ODs) such that a first delivery catheter 80 slidingly receives a second delivery catheter 80 (e.g. the second delivery catheter OD is less than or equal to the first delivery catheter ID), and the second delivery catheter 80 slidingly receives a third delivery catheter 80 (e.g. the third delivery catheter OD is less than or equal to the second delivery catheter ID), and so on. In these configurations, the first delivery catheter 80 (e.g. its distal end) can be advanced to a first anatomical location, the second delivery catheter 80 (e.g. its distal end) can be advanced through the first delivery catheter to a second anatomical location distal or otherwise remote (hereinafter “distal”) to the first anatomical location, and so on as appropriate, using sequentially smaller diameter delivery catheters. In some embodiments, delivery catheter 80 can be of similar construction and arrangement to the similar components described in applicant's co-pending U.S. patent application Ser. No. 17/350,021 (Docket No. GTY-002-US-CON2), titled “Imaging System Includes Imaging Probe and Delivery Devices”, filed Jun. 17, 2021.

In some embodiments, delivery catheter 80 comprises a guide extension catheter, such as a catheter including a coil-reinforced hollow shaft, and a push wire attached to the proximal end of the shaft. The shaft can include a skived (partial circumferential) proximal portion for ease of insertion of a separate device (e.g. a treatment device and/or probe 100) through the shaft.

Rotation assembly 210 operably attaches to connector assembly 150 of imaging probe 100. Rotation assembly 210 can comprise one or more rotary joints, optical connectors, rotational actuators (e.g. motors), and/or linkages, configured to operably attach to, allow the rotation of, and/or cause the rotation of optical core 110. Connector assembly 150 can be constructed and arranged to removably attach to rotation assembly 210, and to allow a rotating connection between proximal end 1101 and a rotating fiber optic joint (such as a fiber optic rotary joint or FORJ). Rotation assembly 210 can be of similar construction and arrangement to similar components described in applicant's co-pending U.S. patent application Ser. No. 16/764,087 (Docket No. GTY-003-US), titled “Imaging System”, filed May 14, 2020, and applicant's co-pending U.S. patent application Ser. No. 17/276,500 (Docket No. GTY-004-US), titled “Imaging System with Optical Pathway”, filed Mar. 16, 2021. Rotation assembly 210 can be configured to rotate optical core 110 at speeds of at least 100 rotations per second, such as at least 200 rotations per second or 250 rotations per second, or at speeds between 20 rotations per second and 1000 rotations per second. Rotation assembly 210 can comprise a rotational actuator selected from the group consisting of: a motor; a servo; a stepper motor (e.g. a stepper motor including a gear box); an actuator; a hollow core motor; and combinations thereof. In some embodiments, rotation assembly 210 is configured to rotate optical assembly 115 and optical core 110 in unison.

Retraction assembly 220 operably attaches to imaging probe 100, such as to retract imaging probe 100 relative to a patient access site. A retraction element 2210 can operably attach to retraction assembly 220 and imaging probe 100, such as to transfer a retraction force from retraction assembly 220 to imaging probe 100. Retraction element 2210 can comprise a conduit 2211, surrounding a linkage 2212, slidingly received therein. Retraction element 2210 can comprise a connector 2213 that operably attaches to retraction assembly 220, such that retraction assembly 220 can retract linkage 2212 relative to conduit 2211. In some embodiments, conduit 2211 comprises a connector 2214 that operably attaches to a reference point near the patient access site, for example to connector 82 of delivery catheter 80, such as to establish a reference for retraction of imaging probe 100 relative to the patient. Connector 2214 can attach to a reference point such as by attaching to a patient introduction device, surgical table, and/or another fixed or semi fixed point of reference. Linkage 2212 releasably attaches to connector 180 of imaging probe 100. Retraction assembly 220 retracts at least a portion of imaging probe 100 (e.g. the portion of imaging probe 100 distal to the attached connector 180) relative to the established reference by retracting linkage 2212 relative to conduit 2211 (e.g. retract a portion of linkage 2212 exiting a portion of conduit 2211, as shown). In some embodiments, retraction assembly 220 is configured to retract at least a portion of imaging probe 100 (e.g. at least optical assembly 115 and a portion of shaft 120) at a rate of between 5 mm/sec and 200 mm/sec, or between 5 mm/sec and 100 mm/sec, such as a rate of approximately 60 mm/sec. Additionally or alternatively, a pullback procedure can be performed during a time period of between 0.5 sec and 25 sec, for example approximately 20 sec (e.g. over a distance of 100 mm at 5 mm/sec). Service loop 185 of imaging probe 100 can be positioned between connector 180, and rotation assembly 210, such that imaging probe 100 can be retracted relative to the patient while rotation assembly 210 remains stationary (e.g. attached to the surgical table and/or to a portion of console 300).

Retraction assembly 220 further comprises a motive element configured to retract linkage 2212. In some embodiments, the motive element comprises a linear actuator, a worm drive operably attached to a motor, a pulley system, and/or other linear force transfer mechanisms. Linkage 2212 can be operably attached to the motive element via one or more linkages and/or connectors. Retraction assembly 220 can be of similar construction and arrangement to similar components described in applicant's co-pending U.S. patent application Ser. No. 16/764,087 (Docket No. GTY-003-US), titled “Imaging System”, filed May 14, 2020.

In some embodiments, PIU 200 can comprise a single discrete component (e.g. a single housing) which can contain both rotation assembly 210 and retraction assembly 220. Alternatively or additionally, PIU 200 can comprise two or more discrete components (e.g. two or more housings), such as a separate component for each of rotation assembly 210 and retraction assembly 220. In some embodiments, connector assembly 150, service loop 185, retraction element 2210, and connector 2213 are included in a single discrete component (e.g. housed within a single housing) and configured to operably attach to both rotation assembly 210 and retraction assembly 220 (e.g. such as when rotation assembly 210 and retraction assembly 220 are housed within a single housing or otherwise included in a single discrete component).

In some embodiments, system 10 includes a supplementary imaging device (e.g. in addition to imaging probe 100), second imaging device 15. Second imaging device 15 can comprise an imaging device such as one or more imaging devices selected from the group consisting of: an X-ray; a fluoroscope such as a single plane or biplane fluoroscope; a CT Scanner; an MRI; a PET Scanner; an ultrasound imager; and combinations of one or more of these. In some embodiments, second imaging device 15 comprises a device configured to perform rotational angiography.

In some embodiments, system 10 includes a device configured to treat the patient (e.g. provide one or more therapies to the patient), treatment device 16. Treatment device 16 can comprise an occlusion treatment device and/or other treatment device selected from the group consisting of: a balloon catheter constructed and arranged to dilate a stenosis or other narrowing of a blood vessel; a drug eluting balloon; an aspiration catheter; a sonolysis device; an atherectomy device; a thrombus removal device such as a stent retriever device; a Trevo™ stentriever; a Solitaire™ stentriever; a Revive™ stentriever; an Eric™ stentriever; a Lazarus™ stentriever; a stent delivery catheter; a microbraid implant; an embolization system; a WEB™ embolization system; a Luna™ embolization system; a Medina™ embolization system; and combinations of one or more of these. In some embodiments, imaging probe 100 and/or another component of system 10 is configured to collect data related to treatment device 16 (e.g. treatment device 16 location, orientation and/or other configuration data), after treatment device 16 has been inserted into the patient.

System 10 can further comprise one or more devices that are configured to monitor one, two, or more physiologic and/or other parameters of the patient, such as patient monitor 17 shown. Patient monitor 17 can comprise one or more monitoring devices selected from the group consisting of: an ECG monitor; an EEG monitor; a blood pressure monitor; a blood flow monitor; a respiration monitor; a patient movement monitor; a T-wave trigger monitor; and combinations of these.

System 10 can further comprise one or more fluid injectors, injector 20 shown, each of which can be configured to inject one or more fluids, such as a flushing fluid, an imaging contrast agent (e.g. a radiopaque contrast agent, hereinafter “contrast”) and/or other fluid, such as injectate 21 shown. Injector 20 can comprise a power injector, syringe pump, peristaltic pump or other fluid delivery device configured to inject a contrast agent, such as radiopaque contrast, and/or other fluids. In some embodiments, injector 20 is configured to deliver contrast and/or other fluid (e.g. contrast, saline, and/or dextran). In some embodiments, injector 20 delivers fluid in a flushing procedure, such as is described herein. In some embodiments, injector 20 delivers contrast or other fluid through delivery catheter 80 comprising an ID of between 5 Fr and 9 Fr, a delivery catheter 80 comprising an ID of between 0.53″ to 0.70″, or a delivery catheter 80 comprising an ID between 0.0165″ and 0.027″. In some embodiments, contrast or other fluid is delivered through a delivery catheter as small as 4 Fr (e.g. for distal injections). In some embodiments, injector 20 delivers contrast and/or other fluid through the lumen of delivery catheter 80, while one or more smaller delivery catheters 80 also reside within the lumen of delivery catheter 80. In some embodiments, injector 20 is configured to deliver two dissimilar fluids simultaneously and/or sequentially, such as a first fluid delivered from a first reservoir and comprising a first concentration of contrast, and a second fluid from a second reservoir and comprising less or no contrast.

Injectate 21 can comprise fluid selected from the group consisting of: optically transparent material; saline; visualizable material; contrast; dextran; an ultrasonically reflective material; a magnetic material; and combinations thereof. Injectate 21 can comprise contrast and saline. Injectate 21 can comprise at least 20% contrast. During collection of image data (e.g. during a pullback), a flushing procedure can be performed, such as by delivering one or more fluids, (e.g. injectate 21 as propelled by injector 20 or other fluid delivery device), to remove blood or other somewhat opaque material (hereinafter non-transparent material) proximate optical assembly 115 (e.g. to remove non-transparent material between optical assembly 115 and a delivery catheter and/or non-transparent material between optical assembly 115 and a vessel wall), such as to allow light distributed from optical assembly 115 to reach and reflectively return from all tissue and other objects to be imaged. In these flushing embodiments, injectate 21 can comprise an optically transparent material, such as saline. Injectate 21 can comprise one or more visualizable materials, as described herein.

As an alternative or in addition to its use in a flushing procedure, injectate 21 can comprise material configured to be viewed by second imaging device 15, such as when injectate 21 comprises a contrast material configured to be viewed by a second imaging device 15 comprising a fluoroscope and/or other X-ray device; an ultrasonically reflective material configured to be viewed by a second imaging device 15 comprising an ultrasound imager; and/or a magnetic material configured to be viewed by a second imaging device 15 comprising an MRI.

System 10 can further comprise an implant, such as implant 31, which can be implanted in the patient via a delivery device, such as an implant delivery device 30 and/or delivery catheter 80. Implant 31 can comprise an implant (e.g. a temporary or chronic implant) for treating, for example, a vascular occlusion and/or an aneurysm. In some embodiments, implant 31 comprises one or more implants selected from the group consisting of: a flow diverter; a Pipeline™ flow diverter; a Surpass™ flow diverter; an embolization coil; a stent; a Wingspan™ stent; a covered stent; an aneurysm treatment implant; and combinations of one or more of these.

Implant delivery device 30 can comprise a catheter and/or other tool used to deliver implant 31, such as when implant 31 comprises a self-expanding or balloon expandable portion. In some embodiments, system 10 comprises imaging probe 100, one or more implants 31 and/or one or more implant delivery devices 30. In some embodiments, imaging probe 100 is configured to collect data related to implant 31 and/or implant delivery device 30 (e.g. implant 31 and/or implant delivery device 30 anatomical location, orientation and/or other configuration data), after implant 31 and/or implant delivery device 30 has been inserted into the patient.

In some embodiments, one or more system components, such as second imaging device 15, treatment device 16, patient monitor 17, injector 20, implant delivery device 30, delivery catheter 80, imaging probe 100, PIU 200, rotation assembly 210, retraction assembly 220, and/or console 300, further comprise one or more functional elements (“functional element” herein), such as functional elements 99a, 99b, 99c, 99d, 99e, 89, 199, 299, 219, 229, and/or 399, respectively, each as shown. Each functional element can comprise at least two functional elements. Each functional element can comprise one or more elements selected from the group consisting of: sensor; transducer; and combinations thereof. The functional element can comprise a sensor configured to produce a signal. The functional element can comprise a sensor selected from the group consisting of: a physiologic sensor; a pressure sensor; a strain gauge; a position sensor; a GPS sensor; an accelerometer; a temperature sensor; a magnetic sensor; a chemical sensor; a biochemical sensor; a protein sensor; a flow sensor such as an ultrasonic flow sensor; a gas detecting sensor such as an ultrasonic bubble detector; a sound sensor such as an ultrasound sensor; and combinations thereof. The sensor can comprise a physiologic sensor selected from the group consisting of: a pressure sensor such as a blood pressure sensor; a blood gas sensor; a flow sensor such as a blood flow sensor; a temperature sensor such as a blood or other tissue temperature sensor; and combinations thereof. The sensor can comprise a position sensor configured to produce a signal related to a vessel path geometry (e.g. a 2D or 3D vessel path geometry). The sensor can comprise a magnetic sensor. The sensor can comprise a flow sensor. The system can further comprise an algorithm configured to process the signal produced by the sensor-based functional element. Each functional element can comprise one or more transducers. Each functional element can comprise one or more transducers selected from the group consisting of: a heating element such as a heating element configured to deliver sufficient heat to ablate tissue; a cooling element such as a cooling element configured to deliver cryogenic energy to ablate tissue; a sound transducer such as an ultrasound transducer; a vibrational transducer; and combinations thereof.

In some embodiments, imaging probe 100 comprises an overall length of at least 120 cm, such as at least 160 cm, such as approximately 280 cm. In some embodiments, imaging probe 100 comprises an overall length of no more than 350 cm. In some embodiments, imaging probe 100 comprises a length configured to be inserted into the patient (“insertable length” herein) of at least 90 cm, such as at least 100 cm, such as approximately 145 cm. In some embodiments, imaging probe 100 comprises an insertable length of no more than 250 cm, such as no more than 200 cm. In some embodiments, distal tip 119 comprises a spring tip with a length of at least 5 mm, such as at least 25 mm, such as approximately 15 mm. In some embodiments, distal tip 119 comprises a spring tip with a length of no more than 75 mm, such as no more than 30 mm. In some embodiments, a distal portion of shaft 120 (e.g. window 130) comprises an outer diameter of less than 2 Fr, such as less than 1.4 Fr, such as approximately 1.1 Fr. In some embodiments, a distal portion of shaft 120 (e.g. window 130) comprises an outer diameter of at least 0.5 Fr, such as at least 0.9 Fr. In some embodiments, shaft 120 comprises one or more materials selected from the group consisting of: polyether ether ketone (PEEK); nylon; polyether block amide; nickel-titanium alloy; and combinations of these.

In some embodiments, at least a portion of imaging probe 100 (e.g. the most flexible portion) is configured to safely and effectively be positioned in a radius of curvature as low as 5 mm, 4 mm, 3 mm, 2 mm, and/or 1 mm. In some embodiments optical core 110 comprises an optical fiber with a diameter of less than 120 μm, such as less than 100 μm, such as less than 80 μm, such as less than 60 μm, such as approximately 40 μm. In some embodiments, optical core 110 comprises a numerical aperture of one or more of 0.11, 0.14, 0.16, 0.17, 0.18, 0.20, and/or 0.25. In some embodiments, optical assembly 115 comprises a lens selected from the group consisting of: a GRIN lens; a molded lens; a shaped lens, such as a melted and polished lens; a lens comprising an axicon structure, (e.g. an axicon nano-structure); and combinations of these. In some embodiments, optical assembly 115 comprises a lens with an outer diameter of less than 200 μm, such as less than 170 μm, such as less than 150 μm, such as less than 100 μm, such as approximately 80 μm. In some embodiments optical assembly 115 comprises a lens with a length of less than 3 mm, such as less than 1.5 mm. In some embodiments, optical assembly 115 comprises a lens with a length of at least 0.5 mm, such as at least 1 mm. In some embodiments, optical assembly 115 comprises a lens with a focal length of at least 0.5 mm and/or no more than 5.0 mm, such as at least 1.0 mm and/or no more than 3.0 mm, such as a focal length of approximately 0.5 mm. In some embodiments, optical assembly 115 can comprise longer focal lengths, such as to view structures outside of the blood vessel in which optical assembly 115 is inserted. In some embodiments, optical assembly 115 has a working distance (also termed depth of field, confocal distance, or Rayleigh Range) of up to 1 mm, such as up to 5 mm, such as up to 10 mm, such as a working distance of at least 1 mm and/or no more than 5 mm. In some embodiments, optical assembly 115 comprises an outer diameter of at least 80 m and/or no more than 200 m, such as at least 150 m and/or no more than 170 m, such as an outer diameter of approximately 150 m. In some embodiments, system 10 (e.g. retraction assembly 220) is configured to perform a pullback of imaging probe 100 at a speed of at least 10 mm/sec and/or no more than 300 mm/sec, such as at least 50 mm/sec and/or no more than 200 mm/sec, such as a pullback speed of approximately 100 mm/sec. In some embodiments, system 10 (e.g. retraction assembly 220) is configured to perform a pullback for a distance of at least 25 mm and/or no more than 200 mm, such as at least 25 mm and/or no more than 150 mm, such as a distance of approximately 50 mm. In some embodiments, system 10 (e.g. retraction assembly 220) is configured to perform a pullback over a time period of at least 0.2 seconds and/or no more than 5.0 seconds, such as at least 0.5 seconds and/or no more than 2.0 seconds, such as a time period of approximately 1.0 second. In some embodiments, system 10 (e.g. rotation assembly 210) is configured to rotate optical core 110 at an angular velocity of at least 20 rotations per second and/or no more than 1000 rotations per second, such as at least 100 rotations per second and/or no more than 500 rotations per second, such as an angular velocity of approximately 250 rotations per second. In some embodiments, delivery catheter 80 comprises an inner diameter of at least 0.016″ and/or no more than 0.050″, such as at least 0.016″ and/or no more than 0.027″, such as an inner diameter of approximately 0.021″.

In some embodiments, console 300 comprises imaging assembly 320 that can be configured to provide light to optical assembly 115 (e.g. via optical core 110) and collect light from optical assembly 115 (e.g. via optical core 110). Imaging assembly 320 can include a light source 325. Light source 325 can comprise one or more light sources, such as one or more light sources configured to provide one or more wavelengths of light to optical assembly 115 via optical core 110. Light source 325 is configured to provide light to optical assembly 115 (via optical core 110) such that image data can be collected comprising cross-sectional, longitudinal and/or volumetric information related to a patient site or implanted device being imaged. Light source 325 can be configured to provide light such that the image data collected includes characteristics of tissue within the patient site being imaged, such as to quantify, qualify or otherwise provide information related to a patient disease or disorder present within the patient site being imaged. Light source 325 can be configured to deliver broadband light and have a center wavelength in the range from 350 nm to 2500 nm, from 800 nm to 1700 nm, from 1280 nm to 1310 nm, or approximately 1300 nm (e.g. light delivered with a sweep range from 1250 nm to 1350 nm). Light source 325 can comprise a sweep rate of at least 20 kHz. In some embodiments, light source 325 comprises a sweep rate of at least 100 KHz, such as at least 200 Khz, 300 KHz, 400 KHz, and/or 500 KHz, for example approximately 200 kHz. These faster sweep rates provide numerous advantages (over similar systems comprising slower sweep rates), such as to provide a higher frame rate, as well as being compatible with rapid pullback and rotation rates. For example, the higher sweep rate enables the requisite sampling density (e.g. the amount of luminal surface area swept by the rotating beam) to be achieved in a shorter time, advantageous in most situations and especially advantageous when there is relative motion between the probe and the surface/tissue being imaged such as arteries in a beating heart. Light source 325 bandwidth can be selected to achieve a desired resolution, which can vary according to the needs of the intended use of system 10. In some embodiments, bandwidths are about 5% to 15% of the center wavelength, which allows resolutions of between 20 μm and 5 μm. Light source 325 can be configured to deliver light at a power level meeting ANSI Class 1 (“eye safe”) limits, though higher power levels can be employed. In some embodiments, light source 325 delivers light in the 1.3 μm band at a power level of approximately 20 mW. Tissue light scattering is reduced as the center wavelength of delivered light increases, however water absorption increases. Light source 325 can deliver light at a wavelength approximating 1300 nm to balance these two effects. Light source 325 can be configured to deliver shorter wavelength light (e.g. approximately 800 nm light) to traverse patient sites to be imaged including large amounts of fluid. Alternatively or additionally, light source 325 can be configured to deliver longer wavelengths of light (e.g. approximately 1700 nm light), such as to reduce a high level of scattering within a patient site to be imaged. In some embodiments, light source 325 comprises a tunable light source (e.g. light source 325 emits a single wavelength that changes repetitively over time), and/or a broad-band light source. Light source 325 can comprise a single spatial mode light source or a multimode light source (e.g. a multimode light source with spatial filtering).

Light source 325 can comprise a relatively long effective coherence length, such as a coherence length of greater than 10 mm, such as a length of at least 50 mm, at all frequencies within the bandwidth of the light source. This coherence length capability enables longer effective scan ranges to be achieved by system 10, as the light returning from distant objects to be imaged (e.g. tissue) must remain in phase coherence with the returning reference light, in order to produce detectable interference fringes. In the case of a swept-source laser, the instantaneous linewidth is very narrow (i.e. as the laser is sweeping, it is outputting a very narrow frequency band that changes at the sweep rate). Similarly, in the case of a broad-bandwidth source, the detector arrangement must be able to select very narrow linewidths from the spectrum of the source. The coherence length scales inversely with the linewidth. Longer scan ranges enable larger or more distant objects to be imaged (e.g. more distal tissue to be imaged). Current systems have lower coherence length, which correlates to reduced image capture range as well as artifacts (ghosts) that arise from objects outside the effective scan range.

In some embodiments, light source 325 comprises a sweep bandwidth of at least 30 nm and/or no more than 250 nm, such as at least 50 nm and/or no more than 150 nm, such as a sweep bandwidth of approximately 100 nm. In some embodiments, light source 325 comprises a center wavelength of at least 800 nm and/or no more than 1800 nm, such as at least 1200 nm and/or no more than 1350 nm, such as a center wavelength of approximately 1300 nm. In some embodiments, light source 325 comprises an optical power of at least 5 mW and/or no more than 500 mW, such as at least 10 mW and/or no more than 50 mW, such as an optical power of approximately 20 mW.

System 10 can comprise one or more operably-connecting cables or other conduits, bus 58 shown. Bus 58 can operably connect PIU 200 to console 300, rotation assembly 210 to console 300 (as shown), retraction assembly 220 to console 300, and/or rotation assembly 210 to retraction assembly 220. Bus 58 can comprise one or more optical transmission fibers, wires, traces, and/or other electrical transmission cables, fluid conduits, and combinations of one or more of these. In some embodiments, bus 58 comprises at least an optical transmission fiber that optically couples rotation assembly 210 to imaging assembly 320 of console 300. Additionally or alternatively, bus 58 comprises at least power and/or data transmission cables that transfer power and/or drive signals to one or more of motive elements of rotation assembly 210 and/or retraction assembly 220.

Console 300 can include processing unit 310, which can be configured to perform and/or facilitate one or more functions of system 10, such as one or more processes, energy deliveries (e.g. light energy deliveries), data collections, data analyses, data transfers, signal processing, and/or other functions (“functions” herein). Processing unit 310 can include processor 312, memory 313, and/or algorithm 315, each as shown. Memory 313 can store instructions for performing algorithm 315 and can be coupled to processor 312. System 10 can include an interface, user interface 350, for providing and/or receiving information to and/or from an operator of system 10. User interface 350 can be integrated into console 300 as shown. In some embodiments, user interface 350 can comprise a component separate from console 300, such as a display separate from, but operably attached to, console 300. User interface 350 can include one, two, or more user input and/or user output components. For example, user interface 350 can comprise a joystick, keyboard, mouse, touchscreen, and/or another human interface device, user input device 351 shown. In some embodiments, user interface 350 comprises a display (e.g. a touchscreen display), such as display 352, also shown. In some embodiments, processor 312 can provide a graphical user interface, GUI 353, to be presented on and/or provided by display 352. User interface 350 can include an input and/or output device selected from the group consisting of: a speaker; an indicator light, such as an LED indicator; a haptic feedback device; a foot pedal; a switch such as a momentary switch; a microphone; a camera, for example when processor 312 enables eye tracking and/or other input via image processing; and combinations of these.

In some embodiments, system 10 includes a data storage and processing device, server 400. Server 400 can comprise an “off-site” server (e.g. outside of the clinical site in which patient image data is recorded), such as a server owned, maintained, and/or otherwise provided by the manufacturer of system 10. Alternatively or additionally, server 400 can comprise a cloud-based server. Server 400 can include processing unit 410 shown, which can be configured to perform one or more functions of system 10, such as one or more functions described herein. Processing unit 410 can include one or more algorithms, algorithm 415. Processing unit 410 can comprise a memory (not shown) storing instructions for performing algorithm 415. Server 400 can be configured to receive and store various forms of data, such as: image data, diagnostic data, planning data and/or outcome data described herein, data 420. In some embodiments, data 420 can comprise data collected from multiple patients (e.g. multiple patients treated with system 10), such as data collected during and/or after clinical procedures where image data was collected from the patient via system 10. For example, image data can be collected via imaging probe 100, recorded by processing unit 310 of console 300, and sent to server 400 for analysis. In some embodiments, console 300 and server 400 can communicate over a network, for example, a wide area network such as the Internet. Alternatively or additionally, system 10 can include a virtual private network (VPN) through which various devices of system 10 transfer data.

As described herein, the one or more functions of system 10 performed by processing unit 310 and/or 410 can be performed by either or both processing units. For example, in some embodiments, image data is collected and preprocessed by processing unit 310 of console 300. The preprocessed image data can then be transferred to server 400, where the image data is further processed. The processed image data can then be transferred back to console 300 to be displayed to the operator (e.g. via GUI 353). In some embodiments, a first set of one or more images (“image” or “images” herein) that is based on a first set of image data (e.g. an image processed locally via processing unit 310) is displayed to the operator following the collection of the image data (e.g. in near-real-time), and a second image based on the first set of image data (e.g. an image processed remotely via processing unit 410) is displayed to the operator subsequently (e.g. the first image is displayed while the second image is being processed).

In some embodiments, algorithm 315 is configured to adjust (e.g. automatically and/or semi-automatically adjust) one or more operational parameters of system 10, such as an operational parameter of console 300, imaging probe 100 and/or a delivery catheter 80. Additionally or alternatively, algorithm 315 can be configured to adjust an operational parameter of a separate device, such as injector 20 and/or implant delivery device 30 described herein. In some embodiments, algorithm 315 is configured to adjust an operational parameter based on one or more sensor signals, such as a sensor signal provided by a sensor-based functional element of the present inventive concepts as described herein. Algorithm 315 can be configured to adjust (e.g. automatically adjust and/or recommend the adjustment of) an operational parameter selected from the group consisting of: a rotational parameter such as rotational velocity of optical core 110 and/or optical assembly 115; a retraction parameter of shaft 120 and/or optical assembly 115 such as retraction velocity, distance, start position, end position and/or retraction initiation timing (e.g. when retraction is initiated); a position parameter such as position of optical assembly 115; a line spacing parameter such as lines per frame; an image display parameter such as a scaling of display size to vessel diameter; an imaging probe 100 configuration parameter; an injectate 21 parameter such as a saline to contrast ratio configured to determine an appropriate index of refraction; a light source 325 parameter such as power delivered and/or frequency of light delivered; and combinations of one or more of these. In some embodiments, algorithm 315 is configured to adjust (e.g. automatically adjust and/or recommend the adjustment of) a retraction parameter such as a parameter triggering the initiation of the pullback, such as a pullback that is initiated based on a parameter selected from the group consisting of: lumen flushing (the lumen proximate optical assembly 115 has been sufficiently cleared of blood or other matter that would interfere with image creation); an indicator signal is received from injector 20 (e.g. a signal indicating sufficient flushing fluid has been delivered); a change in image data collected (e.g. a change in an image is detected, based on the image data collected, that correlates to proper evacuation of blood from around optical assembly 115); and combinations of one or more of these. In some embodiments, algorithm 315 is configured to adjust a system 10 configuration parameter related to imaging probe 100, such as when algorithm 315 identifies (e.g. automatically identifies via an RF or other embedded ID) the attached imaging probe 100 and adjusts a system 10 parameter, such as an optical path length parameter, a dispersion parameter, a catheter-type parameter, an “enabled-feature” parameter (e.g. a parameter that locks and/or unlocks the use of a feature of system 10), a calibration parameter (such as an optical length to physical length conversion parameter), and/or other parameter as listed above. In some embodiments, console 300 is configured to record one or more metrics associated with the performance of imaging probe 100, such as a brightness score. These metrics can be encoded onto probe 100 during use (e.g. encoded into an onboard memory of probe 100, such as onto a writeable RFID tag). Additionally or alternatively, fault information can be encoded onto probe 100 (e.g. written onto an RFID tag), such as when a fault occurs and/or is detected by system 10. For example, fault information can include date and time of image loss, and/or other diagnostic information, such as inability to calibrate.

In some embodiments, algorithm 315 is configured to trigger the initiation of a pullback based on a time-gated parameter. In some embodiments, a T-wave trigger (e.g. provided by a separate device) can be provided to console 300 to begin pullback when the low-motion portion of the heart cycle is detected. As an alternative to a T-wave trigger, or in addition to it, motion patterns (e.g. relative motion patterns) can be tracked (e.g. using angiography) between one or more portions (e.g. components or other features) of imaging probe 100 and relatively stable (e.g. non-moving) portions of the patient's anatomy (e.g. ribs, sternum and/or spinal column).

When a console 300 of system 10 is first installed at a clinical site (e.g. a catheter lab), a calibration routine can be performed, such as a calibration routine used to establish the latency between an angiographic system (e.g. second imaging device 15) of the clinical site and other components of system 10. Essentially, an imaging probe 100 is provided, an angiographic system at the clinical site is engaged, and an angiographic image feed is provided to console 300 (e.g. using any standard video connection, analog or digital). Angiographic system-provided video frames are registered according to a clock of console 300, which is used as a reference time frame. A pullback (e.g. in a patient or in a non-patient simulation mode) of imaging probe 100 is initiated (also coordinated by the console 300 clock) and captured by angiography (e.g. device 15). A trained operator (e.g. a clinician and/or technician) can review the angiographic image frames and designate the first frame in which motion was detected. This process establishes the associated latency according to the console 300 clock. The motion detection can also be automated, for example using a neural network or other algorithm (e.g. of algorithm 315 and/or 415) trained to recognize imaging probe 100 movement (e.g. movement of a marker band of imaging probe 100) under angiography.

In some embodiments, a calibration procedure to establish the latency between an angiographic system (e.g. second imaging device 15) and other components of system 10, and an imaging procedure performed during relatively low motion of a heart cycle, includes the following steps. In a first step, angiography is initiated once probe 100 has been inserted into the patient and deployed into the target anatomy. In a second step, system 10 analyzes the relative motion between one or more portions of imaging probe 100 (e.g. motion of a marker band or other imaging probe 100 portion which follows the beating heart of the patient) and more stable features in the image, such as images of the sternum or spinal column. Once a cardiac rhythm has been established and the low motion portion identified (typically 5-10 heart cycles are used for this analysis, which can be velocity vector analysis, neural network analysis, and the like), an indicator is provided, and a system 10 “metronome” is started. System 10 can reference the output of the metronome, such as at the time that radiopaque flushing material is injected to clear the blood from the target area to be imaged, since the one or more portions of imaging probe 100 (e.g. one or more marker bands) can become radio-invisible during this flushing period (e.g. radiopaque portions of probe 100 cannot be differentiated from the flushing material). In an alternative embodiment, a non-radiopaque flushing material can be used (e.g. dextran). In a third step, flushing is started, such as by an operator or in an automated way controlled by system 10. The flushing continues over several heart cycles, such as 3-5 heart cycles. In a fourth step, clearing of the vessel to be imaged is detected by system 10 analyzing one or more of the images produced by system 10. In a fifth step, at the low motion part of the metronome (e.g. a predicted low motion portion of the heart cycle), and accounting for the latency between system 10 components and the angiographic system previously established, a pullback starts. In some embodiments, the pullback will finish in about one-half of a heart cycle or less, such as to cause capture of all or a portion of image data to remain within the low motion portion of the heart cycle. System 10 can be configured to provide a pullback speed of at least 50 mm/sec, such as at least 100 mm/sec, or 200 mm/sec. In a sixth step, the pullback sequence of images, which include minimal motion artifacts, can be provided to the operator and/or used for: CFD calculations (described herein), implant (e.g. stent) length measurements, and the like. The use of image capture during low motion, as described herein, avoids or at least reduces errors associated with motion artifacts, notably longitudinal motion artifacts.

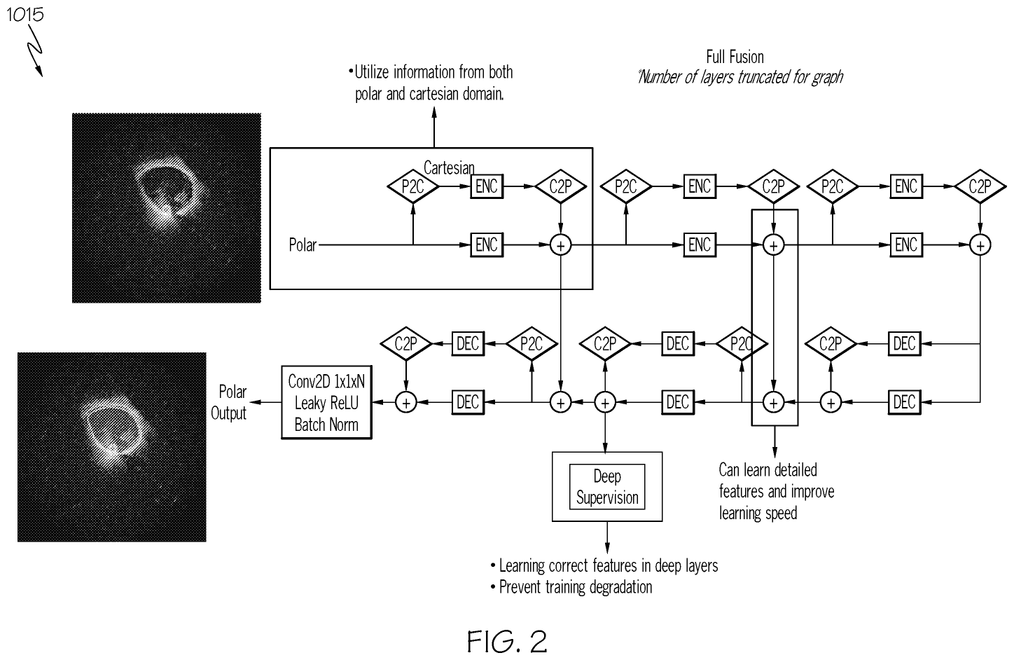

In some embodiments, algorithms 315 and/or 415 (“algorithm 315/415” herein) are configured to perform various image processing of the image data produced by system 10. Algorithm 315/415 can comprise one, two, or more artificial intelligence algorithms configured to perform the various image processing and/or other calculations, as described herein. For example, algorithm 315/415 can comprise neural networks implemented using features of DDNet and/or UNet methodologies, such as features tailored for the processing and segmentation of intravascular image data. In some embodiments, algorithm 315/415 can comprise one or more algorithms of similar configuration as the algorithm described herein in reference to FIG. 2.

System 10 can be configured to allow an operator to modify one or more algorithms of algorithm 315/415. In some embodiments, algorithm 315/415 can comprise one or more biases, such as a bias toward a false positive or a false negative. In some embodiments, algorithm 315/415 comprises a bias toward more accurately identifying larger side-branches at the cost of misidentifying smaller side-branches, as described herein. In some embodiments, system 10 is configured to allow an operator to create and/or modify (e.g. via user interface 350) a bias of one or more algorithms of algorithm 315/415.

Algorithm 315/415 can comprise one or more algorithms that are configured to perform one or more image processing applications selected from the group consisting of: an image quality assessment; procedural device segmentation, such as guide catheter and/or guidewire segmentation; implant segmentation, such as segmentation of endovascular implants such as stents and/or flow-diverters; lumen segmentation, such as segmentation of a vascular lumen; segmentation of side-branches; tissue characterization, such as a characterization of atherosclerotic versus normal; detection of thrombus; and combinations of these.

In some embodiments, algorithm 315/415 comprises various signal and/or image processing algorithms configured to process and/or analyze image data collected by system 10. Using these algorithms, system 10 can be configured to perform an automated quantification of one or more parameters, such as one or more patient parameters (e.g. parameters relating to the health of the patient), one or more image parameters (e.g. parameters relating to the quality of the image data), one or more treatment parameters (e.g. parameters relating to the clinical efficacy and/or technical proficiency of a treatment performed), and combinations of these. For example, system 10 can comprise a metric (e.g. a variable), data metric 525 shown, which can comprise a calculated result that is calculated using, and/or otherwise based on an analysis (e.g. a mathematical analysis) of these various parameters.

Data metric 525 can represent a quantification of the quality of image data, such as a quantification determined by an automated process of system 10. In some embodiments, data metric 525 can comprise a “confidence metric” that represents the quality of the results of an image processing step (e.g. a segmentation process). A data metric 525 comprising a confidence metric can represent a calculated level of accuracy of the image data as determined by system 10 (i.e. the level of “confidence” with which an operator of system 10 can have in the data being presented). In some embodiments, when data metric 525 comprises a confidence metric below a first threshold value (e.g. a value indicating low confidence), system 10 alerts the operator, such as via an indicator displayed to the operator via GUI 353. Additionally or alternatively, system 10 can be configured to not display any image data if a confidence metric related to that image data is below a second threshold value (e.g. a value indicating less confidence than the first threshold value). In some embodiments, system 10 can be configured to display to the operator an alert (e.g. a low confidence data warning) and/or prompt the operator to allow the display of the low confidence image data.

In some embodiments, data metric 525 comprises a quantification of one or more characteristics (e.g. level of apposition or amount of protrusion) describing the interaction between the patient's anatomy and a treatment device (e.g. implant 31) that has been implanted in the patient. For example, system 10 can be configured to analyze image data collected prior to, during, and/or after implantation of an implant, and to determine one or more values of data metric 525 that represent (e.g. correspond to) the interaction between the implant and patient tissue (e.g. the vessel wall, the ostium of one or more side-branches, and/or the neck of one or more aneurysms).

In some embodiments, data metric 525 comprises a metric relating to the healing proximate an implantation site, for example when system 10 is used to collect image data from an implantation site in a follow-up procedure, such as a procedure performed at least one month, at least six months, or at least one year from the implant procedure.

In some embodiments, data metric 525 comprises a metric relating to a predicted outcome of an interventional procedure, such as a metric whose value is calculated and/or updated during the interventional procedure, after the interventional procedure, or both. For example, data metric 525 can be used to provide guidance to the operator by indicating the predicted outcomes of intended (e.g. future) and/or already performed interventions (e.g. based on an analysis of the potential efficacy of the intervention), such as interventions configured to treat brain aneurysms and/or ischemic strokes. For example, the mesh density of a flow diverter covering the neck of an aneurysm can be estimated by system 10 (e.g. based on automated image processing described herein). The mesh density can be used to predict the outcome of the intervention (e.g. long-term dissolution of the aneurysm). Additionally or alternatively, the geometry of the mesh can be used to estimate the angle of optical assembly 115 relative to the surface of the mesh, and to correct the mesh density accordingly. For example, in a bend, the light exiting optical assembly 115 (e.g. the beam of light being transmitted from optical assembly 115) may be along an oblique angle to the mesh surface normal. In this scenario, the mesh pattern will be elongated in the plane of incidence (e.g. the plane defined by the surface normal and the light beam) according to the angle of the light beam. Correcting this elongation to achieve a symmetric pattern can provide the angle of the light beam, and this angle information can be used by system 10 to correct the calculated density of the mesh.

In some embodiments, data metric 525 comprises a metric that informs (e.g. its value is used to recommend or otherwise inform) the patient's clinician to potentially perform an additional (e.g. second) therapeutic procedure on the patient, such as to optimize or at least improve the therapeutic treatment in which at least a first procedure (e.g. an interventional procedure) has already been performed. The additional therapeutic procedure can comprise an interventional procedure selected from the group consisting of: an adjustment to a device (e.g. treatment device 16) implanted in the patient in a previous procedure, such as an adjustment comprising a repositioning, expansion, contraction, and/or other adjustment to the implant; implantation of a device (e.g. device 16) into the patient, whether or not a previous device had been implanted in the patient; a vessel dilation procedure; an atherectomy procedure and/or other procedure in which occlusive material is removed; a coiling or other procedure in which undesired space within the vascular system is occluded; a drug-delivery procedure; and combinations of these.

In some embodiments, system 10 can identify if a myocardial bridge exists over a portion of an imaged vessel. For example, system 10 can automatically detect the presence of a myocardial bridge (e.g. via algorithm 315/415), and/or the data presented to the operator of system 10 can indicate the presence of a myocardial bridge (e.g. such that the operator can draw conclusions based on the data presented). In some embodiments, image data can be collected by system 10 during a pullback procedure in which imaging probe 100 is retracted at a speed in which multiple heart cycles are captured during the pullback, such that the strain on the imaged vessel (e.g. strain caused by motion of the heart) can be analyzed throughout the heart cycle. In some embodiments, system 10 is configured to identify a myocardial bridge by analyzing image data to detect an artifact in the image data indicating the presence of a myocardial bridge (e.g. a signature artifact, similar to an echolucent “halo” that can be seen when imaging a myocardial bridge using intravascular ultrasound).

In some embodiments, system 10 is configured to quantify the quality of image data, such as a quantification determined by an automated process of system 10, such as is described herein. In some embodiments, if the quality of the image data is below a threshold, one or more analytic processes of system 10 (e.g. image analysis described herein) may be disabled, such that the process is not performed on poor quality image data. For example, if image data is analyzed and it is determined (e.g., by system 10 and/or an operator of system 10) that optical assembly 115 started and/or ended within a stent during a pullback procedure, system 10 can be configured to disable subsequent CFD or other calculations described herein based on that poor image data. In some embodiments, system 10 can assess the quality of a purge procedure based on the quality of the image data. For example, system 10 can assess image quality to identify blood ingress into delivery catheter 80, and indicate the need to purge. This analysis can be used for providing feedback to the user in real-time during imaging, such as by displaying a warning message (e.g. “purge catheter”). Similarly, after an image acquisition is completed, system 10 can analyze the image data and display a warning to the user if catheter purge was incomplete. In some embodiments, system 10 can analyze image data to identify blood residuals in the lumen, and to display a warning to the user as well as indicate to the user areas where blood clearance is incomplete. If blood clearance is incomplete in the region of high interest for CFD calculation (such as obscuring frame of reference or a stenosis), a warning can be provided about insufficient image quality for a CFD calculation.

In some embodiments, system 10 is configured to perform various computational fluid dynamics (CFD) and/or optical flow ratio (OFR) calculations using high-resolution image data (e.g. OCT image data) to accurately simulate blood flow in a stenosed artery (e.g. a coronary artery), and to estimate pressure drops through one or more lesions, such as is described herein. These methods provide the user (e.g. interventionalists) with a combined and simultaneous measurement of arterial anatomy and vessel hemodynamic conditions (e.g. “Physio-Anatomy”) in high-resolution, that can be used to better characterize and diagnose the significance of stenosed coronary arteries pre-intervention, as well as following intervention (e.g. post-intervention). This information can be used to provide informed guidance and/or to optimize intervention steps as detailed herein.

Traditional clinical practice is limited to the use of either intravascular imaging (e.g. OCT or IVUS imaging) or physiology measurements (e.g. FFR, iFR, RFR, etc.) at one time, as imaging and physiology measurements can only be acquired using separate instruments (e.g. single purpose catheters). System 10 can be configured to enable capture (e.g. in a single “pullback acquisition”) of both vessel anatomy and physiology. This combined solution has the key advantage of providing intrinsically co-registered anatomy and physiology data (e.g. data captured with a single device), that can be used to better plan and optimize coronary interventions than any of these tools alone.

In some embodiments, CFD simulations performed by system 10 are designed to closely simulate hyperemic conditions, for example as it is done for the acquisition of fractional flow reserve data using a pressure wire. Alternatively or additionally, CFD methods can be used to simulate non-hyperemic conditions, for example similar to the way iFR or RFR catheters are used to collect vessel hemodynamic data. FFR devices typically make a single FFR measurement from a single location distal to all lesions. Using CFD methods of system 10 described herein, blood flow and pressure drops can be more easily evaluated for the entire coronary segment imaged with OCT.

Traditional FFR methodologies suffer from a major limitation due to lesion crosstalk. For example in the case of serial lesions, an FFR acquisition is unable to discern the individual contribution of each lesion. The CFD methods of system 10 described herein will be able to determine the contribution of each lesion, indicating which of the imaged lesions is more significant and which to treat.

System 10 can be configured to achieve a CFD simulation and pressure drop evaluation of a whole arterial segment (e.g. 100 mm or more) in a few seconds (e.g. less than 20 sec) using a simplified quasi-2D and/or 2D solver. If compared to a “full” 3D solver (e.g. a solver configured to implement Navier Stokes equations), a quasi-2D and/or 2D solver allows for an order of magnitude or more reduced computational time, retaining sufficient accuracy for coronary pressure drop evaluation.

In some embodiments, CFD simulations heavily rely on the segmentation of image data (e.g. OCT image data). Segmentation can be obtained through traditional image processing algorithms and/or AI methodologies (e.g. machine learning, deep learning, neural network, and/or other artificial intelligence methodologies). In some embodiments, these methodologies include the various steps of Method 1000, described in reference to FIG. 5 herein, to analyze image data sets (e.g. OCT image data sets) to quantify blood flow and/or pressure drops.

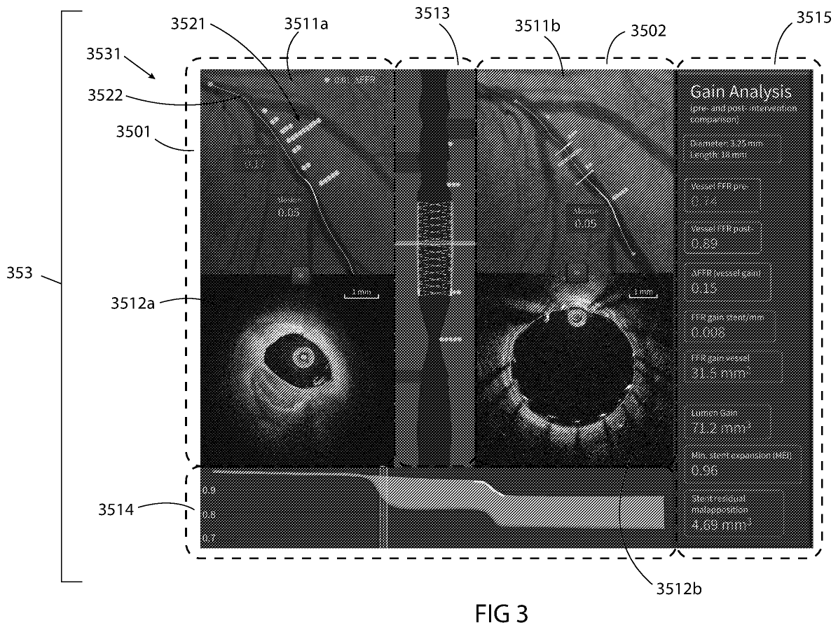

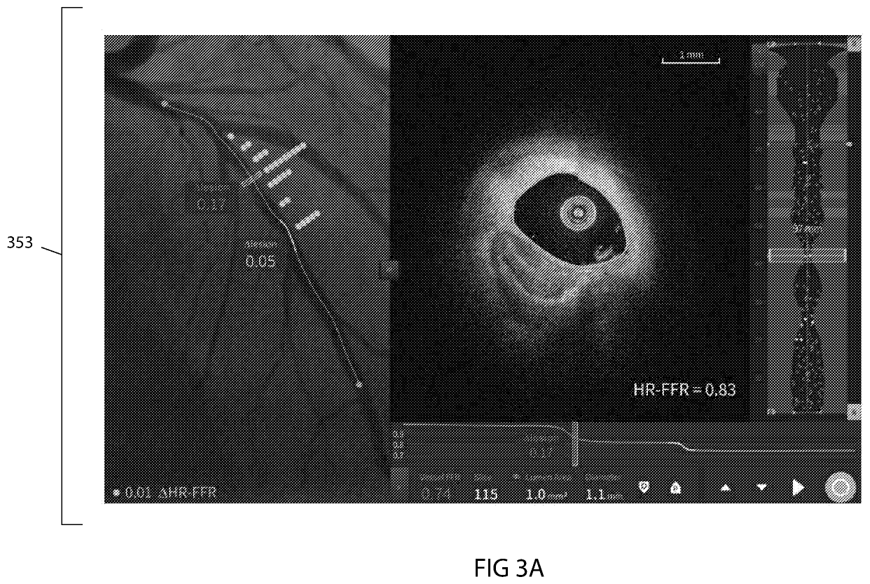

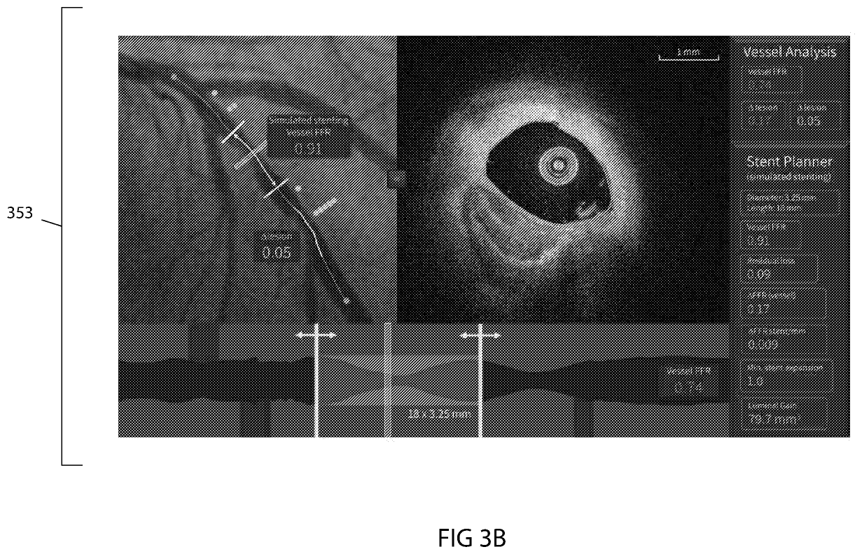

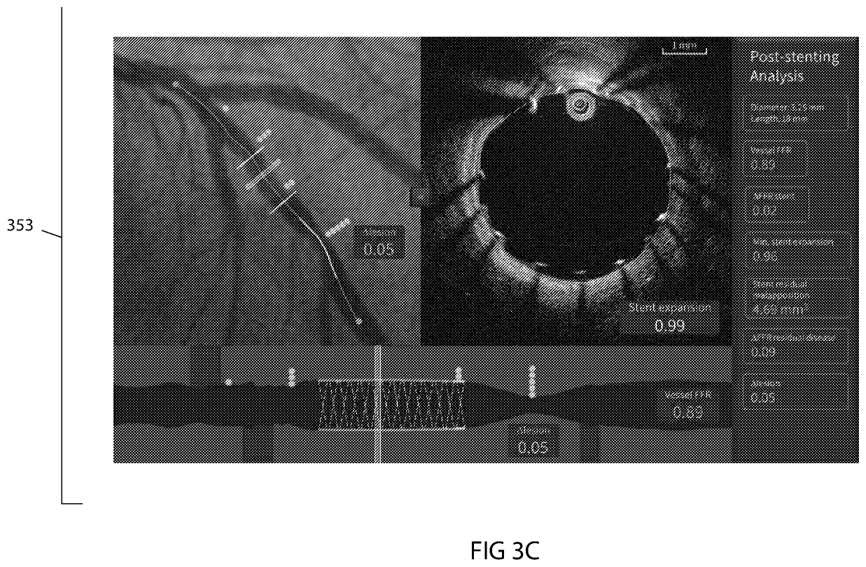

In some embodiments, system 10 comprises a graphical user interface, such as GUI 353 described herein, for example in reference to FIGS. 3A-C. In some embodiments, the GUI is configured to provide the user with an easy and immediate way to obtain and use OCT images and/or simulated physiology data to diagnose coronary stenoses, plan, and optimize coronary interventions. In some embodiments, OCT-FFR “Physio-Anatomy” data can be registered to coronary angiography data to provide a comprehensive tool for interventionalists to accurately plan and guide coronary procedures. Additionally or alternatively, OCT-FFR simulations can be used to create a virtual stenting tool that allows the user of system 10 (e.g. an interventionalist) to simulate the effect of stents of different lengths and diameters over different vessel locations to optimize stent sizing and selection and devise an optimal intervention strategy.

In some embodiments, Physio-Anatomy data can be quantified (e.g. by system 10) by the means of several metrics. For example, these metrics can be used to quantify the effect of the treatment pre-intervention vs. post-intervention (e.g. a “gain” quantification).

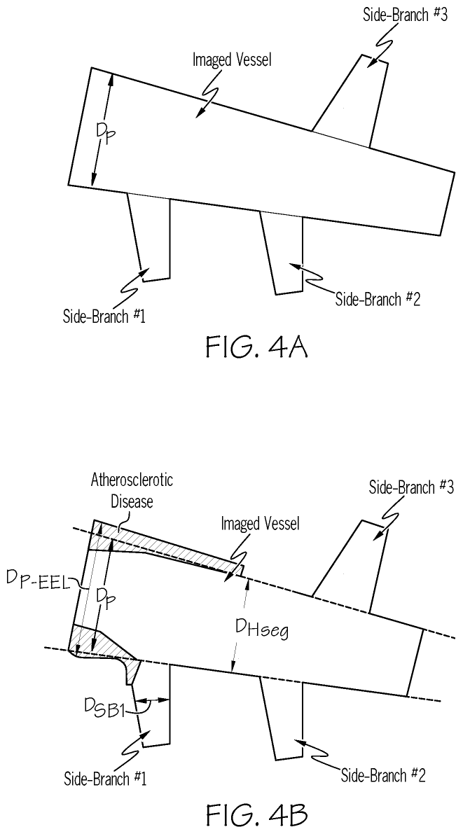

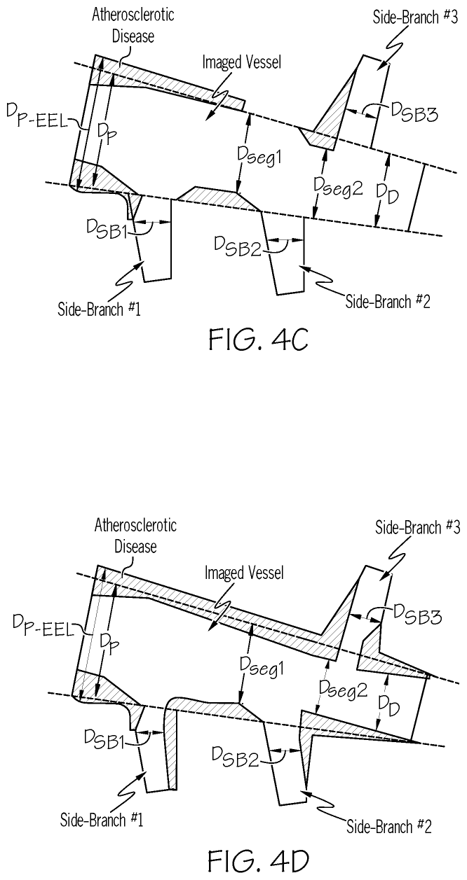

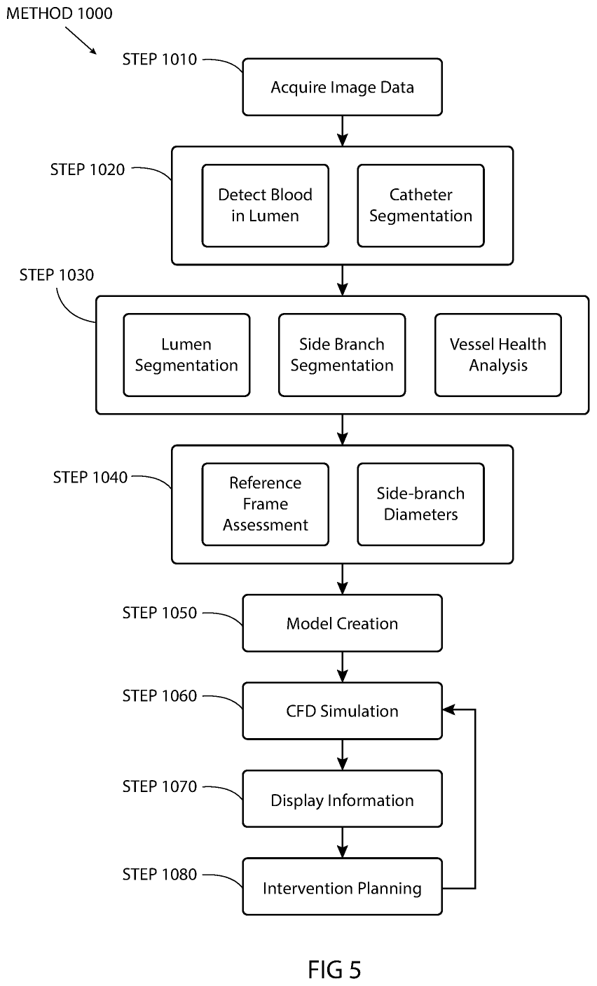

In some embodiments, system 10 is configured to ensure data quality and suitability for CFD calculations. For example, system 10 can be configured to ensure confidence of segmentation results (e.g. side-branch and/or lumen segmentation), by determining a “confidence metric”, such as is described herein. The goal of a confidence metric is to inform the user about potential images with reduced quality where segmentation results are uncertain, allowing for a quick visual review and correction (if needed). In some embodiments, system 10 can be configured to ensure that a complete pullback has been acquired, from a location distal to a lesion to the tip of the guide catheter. In some embodiments, a complete pullback can be defined as: a pullback that captured the entire disease; a pullback that did not start and/or end on a diseased vessel segment; and, if a stent is present, it is imaged in its entirety. If a pullback starts and ends on diseased vessel segments, system 10 can be configured to recover from this situation and provide an accurate CFD measurement. For example, in this scenario, system 10 can identify (e.g. via one or more methodologies described herein) healthy vessel segments and can be configured to use branching laws to estimate vessel diameters and/or areas in proximal and/or distal reference frames, for example as described in reference to FIGS. 4A-4D herein.

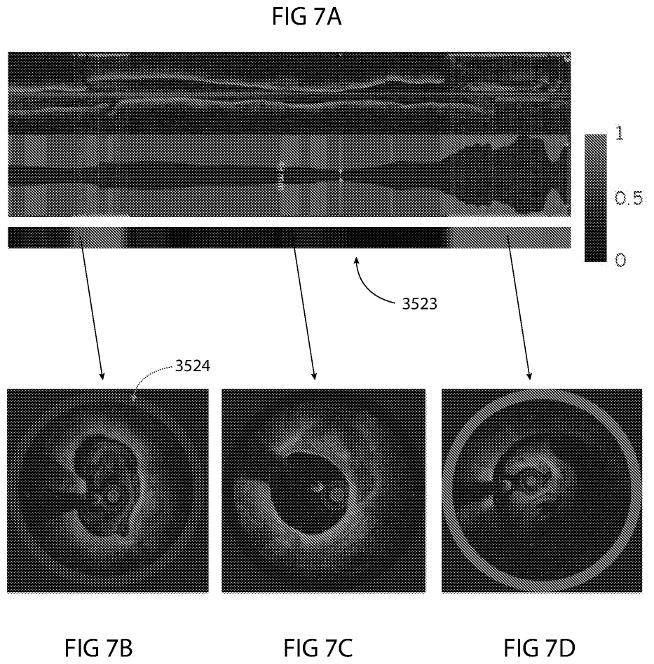

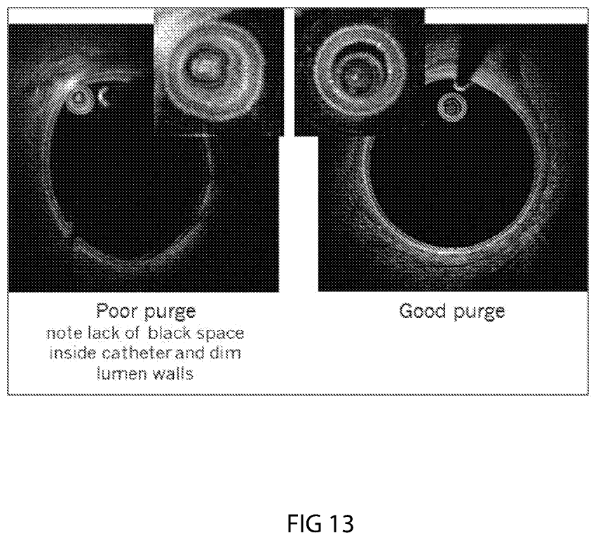

In some embodiments, system 10 is configured to perform an assessment of image quality comprising an assessment of the presence of significant blood residuals in the vessel lumen during a pullback, for example blood that obscures one or more portion of the vessel. System 10 can be configured to perform an assessment as described in reference to FIGS. 7A-7D herein. In some embodiments, system 10 is configured to assess blood that is trapped within a portion of a catheter that is configured to be imaged through (e.g. a portion of a catheter that is configured to be purged with saline before and/or during a pullback), where the trapped blood degrades the image quality. An example of incomplete catheter purging and its effects is shown in FIG. 13 described herein. One or more algorithms of system 10 can be configured to automatically detect degradation in image quality as well as the degree of quality loss, and to warn the user about the poor image quality and potential need to repeat acquisition (e.g. to repeat the pullback). In some embodiments, system 10 is configured to capture one or more angiography images. Analysis of angiography data performed by system 10 can reveal the presence of any significant collateral vessels which may affect the flow of blood within the vessel being imaged, such as one or more “donor” vessel from which blood flows into the vessel being imaged, and/or one or more “recipient” vessels into which blood flows from the vessel being imaged. When significant collateral vessels are present, FFR and CFD calculations might be inaccurate (e.g., falsely low FFR being indicated when the vessel being imaged is a donor vessel having one or more recipient collateral vessels and/or falsely high FFR being indicated when the vessel being imaged is a recipient vessel having one or more donor collateral vessels). In some embodiments, a warning message can be displayed to the user to inform about presence of collateral vessels before a CFD calculation is performed by system 10 and/or before the results are displayed by system 10 to the user.

In some embodiments, system 10 is configured to use various image processing techniques (e.g. as described herein) to help prevent incomplete and/or low-quality image data that can reduce the accuracy of CFD simulations for pressure-drop calculations. An automated determination of image data quality can warn the user of system 10 about potential issues, can help the user in correcting some issues where possible (e.g. help and/or enable the user to fix inaccurate segmentation results), and/or can indicate to the user when a new image data acquisition might be necessary. Automated assessment of data quality can warn and/or provide guidance to the user about moderate quality images and facilitate corrections. Alternatively or additionally, a severe loss of image data quality that cannot be recovered can be displayed to the user, and system 10 can provide guidance on how to improve the image quality (e.g. direct the user to better purge the catheter, and/or to better engage the coronary ostium with the guide catheter) and perform an additional image acquisition.

In some embodiments, system 10 can determine reference diameters (e.g. proximal and distal reference diameters) as well as the size of side-branches (e.g. as described in reference to FIGS. 4A-D herein) and can use this information to calculate an “ideal” and/or “reference” vessel profile to better guide intervention and/or to quantify “stent expansion”. An ideal vessel profile is a metric that can inform a more accurate stent sizing. Stent expansion is a metric that can inform additional steps to optimize stent implantation procedures.

Information collected and/or analyzed by system 10 can be used to provide various functions in a clinical environment. For example, system 10 can be used as a tool to provide training, such as training to a clinician or other user of system 10, and/or can provide equipment diagnostics information in a clinical setting, such as self-diagnostic information and/or diagnostic information related to equipment in the clinical setting that is not a part of system 10. When used in a training scenario, system 10 can be configured to perform an initial and/or periodic assessment of the user of system 10, for example by comparing determinations made by the user (e.g. based on image data gathered by system 10 and input into system 10), to determinations made by system 10 (e.g. by algorithm 315/415) based on similar data (e.g. the same data). For example, system 10 can perform an automated image assessment (e.g. to determine if blood is present during imaging, if a guide catheter is properly positioned during imaging, and/or if a catheter lumen was sufficiently purged during imaging). Based on the automated assessment, system 10 can provide feedback to the user based on the user's operation of system 10 and/or the user's interpretation of the data. For example, system 10 can suggest IQ improvement, provide considerations based on the image quality assessment, and/or provide an overall pullback review.

When used in a diagnostic scenario, system 10 can perform an image quality assessment, and infer (e.g. via algorithm 315/415) from the image quality if a component of system 10 may be the cause of poor image quality. For example, system 10 can detect serviceable issues such as a failing imaging assembly 320 (e.g. from dim image data), poorly connected and/or broken connectors, and/or poor image registration (e.g. caused by NURD or other physical conditions of the catheter). In some embodiments, system 10 is configured to track the usage of various components of the system, for example the number of pullbacks for which an imaging probe 100 and/or an imaging assembly 320 has been used. In some embodiments, system 10 is configured to analyze a first set of image data collected by system 10, as well as a second set of image data from another imaging device (e.g. second imaging device 15), and to analyze (e.g. via algorithm 315/415) the image quality of the second set of image data, such as to provide a diagnostic report of the second imaging device (e.g. to determine if the second device is working properly or is in need of service and/or calibration).

In some embodiments, system 10 is configured to perform an automated review of image data gathered by the system to ensure the image quality is sufficient to perform subsequent calculations based on the image data (e.g. FFR calculations described herein). System 10 can be configured to identify various issues from image data, such as issues selected from the group consisting of: blood in the image, such as caused by inadequate blood clearing; reduced lumen wall confidence; image distortion, such as distortion caused by NURD; lack of guide catheter visualization; insufficient pullback distance, such as less than 40 mm; improper beginning and/or ending points of image data (e.g. starting and/or ending within a stent); and combinations of these.

In some embodiments, system 10 is configured to analyze image data to determine if the patient meets any exclusion criteria (e.g. such that the patient would be excluded from further treatment and/or diagnosis by system 10). Exclusion criteria identified by system 10 can include: presence of a chronic total occlusion (CTO) in the target vessel; severe diffuse disease in the target vessel (e.g. defined as the presence of diffuse, serial gross luminal irregularities present in the majority of the coronary tree); presence of myocardial bridge (MB); target lesion involves the Left Main (e.g. stenosis >50%); an artifact observed in a pre-PCI OCT image for the target lesion or in the event of multiple target lesions; an artifact observed in a pre-PCI OCT image for all target lesions; presence of a target lesion that will need to go through any preparation (including but not limited to balloon dilatation, atherectomy, and the like) prior to pre-PCI OCT imaging and physiology measurement, or in case of multiple target lesions, all target lesions that will need to go through any preparation (including but not limited to balloon dilatation, atherectomy, etc.) prior to pre-PCI OCT imaging and physiology measurement; target lesion and/or significant coronary artery disease (CAD) beyond 60 mm from coronary ostium (e.g. inability to image lesion with OCT in one pullback); incorrect and/or otherwise unsuccessful catheter purge and/or contrast flush; presence of plaque rupture and/or intravascular hematoma in target vessel (visual % diameter stenosis >40%); and combinations of these.

In some embodiments, system 10 is configured to analyze angiography image data to identify a vessel within which imaging probe 100 is positioned (e.g. which vessel image data collected by system 10 represents). In some embodiments, one or more algorithms of system 10 (e.g. algorithm 315/415) is modified based on the vessel being imaged (e.g. automatically modified based on the identification of the vessel being imaged from angiography image data). In some embodiments, system 10 is configured to perform motion correction of OCT image data by analyzing velocity vectors of angiographic image data collected simultaneously with the OCT image data.

In some embodiments, the image processing methodologies of system 10 described herein are configured to automatically perform a process selected from the group consisting of: identify normal and diseased segments of an imaged vessel; identify ideal reference frames for vessel sizing (e.g. to avoid placing a reference segment in a diseased area); optimize scaling laws by avoiding diseased segments as reference diameters; optimize vessel size estimation; and combinations of these.

Referring now to FIG. 2, a graphical representation of a neural network is illustrated, consistent with the present inventive concepts. FIG. 2 depicts an algorithm configured as a neural network, algorithm 1015. Algorithm 315 and/or 415 described herein can each comprise an algorithm that is configured similarly to algorithm 1015 (e.g. when algorithm 1015 is processed by processing unit 310 of console 300 and/or by processing unit 410 of server 400, respectively). In some embodiments, algorithm 315 comprises algorithm 1015 and/or algorithm 415 comprises algorithm 1015. In some embodiments, algorithm 1015 comprises a machine learning, deep learning, neural network, and/or other artificial intelligence algorithm (“AI algorithm” herein) that has been trained by a first processing unit (e.g. processing unit 410) and is configured to process data (e.g. image data) using a second processing unit (e.g. processing unit 310 of console 300). System 10 can be configured to allow an operator to modify one or more algorithms of algorithm 1015. In some embodiments, algorithm 1015 comprises a bias, such as a bias toward a particular result, such as a bias toward a false positive, a bias toward a false negative, or other bias. In these embodiments, system 10 can be configured to allow an operator to create and/or modify (e.g. via user interface 350) a bias of one or more algorithms of algorithm 1015.

Algorithm 1015 can process image data in multiple domains, for example in both polar and cartesian image domains. In some embodiments, algorithm 1015 processes data in two, three, or more image domains. Algorithm 1015 can be configured to process image data in multiple domains by performing image data conversions at each encoding and/or decoding step of algorithm 1015. In some embodiments, algorithm 1015 only requires input of image data in a single image domain, and algorithm 1015 converts the image data from the single domain into one or more additional domains. Algorithm 1015 can be configured to process image data in one or more image domains selected from the group consisting of: the polar domain; the cartesian domain; the longitudinal domain; the en-face image domain; a domain generated by calculating image features, such as first and/or second order features, image texture, image entropy, homogeneity, correlation, contrast, energy, and/or any other image feature; and combinations of these.

In some embodiments, algorithm 1015 is configured to prevent training degradation, including overfitting and improve network generalization.

In some embodiments, algorithm 1015 is configured to learn detailed features (e.g. detailed features of image data). In these embodiments, the learning speed of algorithm 1015 can be improved (e.g. by skipping layers). For example, algorithm 1015 can comprise an algorithm that has been trained to perform at least one process, where the training was completed in less than one week, such as less than one day, such as less than 12 hours.

In some embodiments, algorithm 1015 comprises multiple AI algorithms, where each of the multiple algorithms are configured (e.g. trained) to perform a single image processing application, for example a first algorithm is trained to perform a lumen segmentation application, and a second algorithm is trained to perform a side-branch segmentation application. Alternatively or additionally, algorithm 1015 can comprise a single algorithm that is trained to perform two, three, or more image processing applications, such as a single algorithm comprising two or more modules, each module trained to perform an image segmentation process. For example, an algorithm 1015 comprising a neural network or other AI algorithm can include modules trained to perform both lumen segmentation and side-branch segmentation. In some embodiments, algorithm 1015 is configured to “skip” one or more layers of its neural network to perform one of multiple trained image processing applications (e.g. each module of algorithm 1015 only uses the layers of the neural network that are required to perform the segmentation). In some embodiments, algorithm 1015 comprises one or more modules configured to quantify key features of the image data, for example image data comprising high resolution, three-dimensional image data. Key features quantified by algorithm 1015 can include: features of the vascular anatomy and/or morphology; the vessel lumen; ostium of one or more side-branches; atherosclerotic disease; ideal lumen profile (e.g. as described herein); ideal stent expansion (e.g. as described herein); and combinations of theses. Algorithm 1015 can comprise an AI or other algorithm that is configured to calculate computational fluid dynamics (“CFD”) of an imaged vessel, for example to quantify blood flow and/or pressure drops along the length of an imaged vessel.

Referring now to FIG. 3, an embodiment of a graphical user interface for displaying image data and guiding vascular intervention is illustrated, consistent with the present inventive concepts. System 10 can comprise a kit of tools for assisting a clinician and/or other operator in planning, performing, and/or assessing a vascular intervention (e.g. a cardiac or a neurovascular intervention). GUI 353 can comprise various display areas (e.g. portions of a display) where information is presented to the operator in an arrangement configured to assist in various aspects of an interventional procedure, such as are described herein. Display areas can be rendered on GUI 353 in various arrangements, “workspaces” herein. FIG. 3 illustrates a workspace arranged to display pre-intervention image data alongside post-intervention image data, workspace 3531. Workspace 3531 comprises a pre-intervention display area, workspace area 3501, and a post-intervention display area, workspace area 3502. GUI 353 can render one or more images (e.g. static and/or video images) of angiographic and/or other two-dimensional projections of data (e.g. non-OCT image data) within one or more display areas, such as display areas 3511a and 3511b shown. Display areas 3511a and 3511b can be rendered in workspace areas 3501 and 3502, respectively, and can display pre-intervention and post-intervention data (e.g. angiographic or other 2D data), respectively. Additionally or alternatively, GUI 353 can render one or more images of luminal cross section data (e.g. OCT data) within one or more display areas, such as display areas 3512a and 3512b shown. Display areas 3512a and 3512b can be rendered in workspace areas 3501 and 3502, respectively, and can display pre-intervention and post-intervention data (e.g. OCT data), respectively.

In some embodiments, GUI 353 can render one or more images representing a vascular lumen profile within one or more display areas, such as display area 3513 shown. Display area 3513 can be rendered on workspace 3531, for example between workspace areas 3501 and 3502, as shown. The lumen profile data shown in display area 3513 can represent pre-intervention and/or post-intervention data. In some embodiments, the lumen profile data comprises data calculated by one or more algorithms of system 10, as described herein, such as a calculation performed on image data collected by probe 100 and/or another component of system 10, also as described herein (e.g. OCT and/or non-OCT image data).

In some embodiments, GUI 353 can render one or more images comprising data graphs within one or more display areas, such as display area 3514 shown. Display area 3514 can be rendered on workspace 3531, for example at a location below workspace areas 3501 and 3502, as shown. Graphs shown in display area 3514 can represent pre-intervention and/or post-intervention data. For example, a graph comparing pre-intervention fractional flow reserve (FFR) data along the length of a vessel to post-intervention FFR data can be illustrated as shown in FIG. 3.

In some embodiments, GUI 353 can display numerical data in one or more display areas, such as display area 3515 shown. Display area 3515 can be rendered on workspace 3531, for example along one side of the workspace as shown. Data displayed in display area 3515 can comprise data calculated from image data collected by system 10, and can be selected from the group consisting of: dimensions of an imaged vessel, such as the length and/or average diameter of the vessel; vessel tapering; pre-intervention FFR; intra-intervention FFR; post-intervention FFR; delta FFR; FFR gain per length (e.g. mm) of stent implanted; FFR gain for the imaged vessel; lumen area gained post-intervention; the minimum expansion index (MEI) for an implanted stent; a value corresponding to stent residual malposition; and combinations of these.

GUI 353 can display one or more overlays relative to the data displayed within the display areas described herein. For example, GUI 353 can display overlay 3521 that visually represents the locations along the length of an imaged lumen where a drop in FFR is determined. Overlay 3521 can comprise multiple unitary indicators (e.g. dots as shown), each indicator representing a delta in the calculated FFR, as shown in display areas 3511a,b and 3513. For example, each dot represented in overlay 3521 can represent a delta of 0.01 FFR (e.g. the calculated FFR). In some embodiments, GUI 353 displays an overlay 3522 that visually indicates the locations in which OCT image data was collected through a vessel (e.g. relative to displayed angiographic image data). Overlay 3522 can comprise a line (as shown) that is rendered relative to 2D image data, as shown in display areas 3511a,b. In some embodiments, the properties (e.g. graphical properties) of the line of overlay 3522 can be varied along its length to represent additional data, for example to represent FFR data along the length of the line. For example, the color of the line can be varied (e.g. red to indicate an unhealthy portion of the imaged vessel, green to indicate a healthy portion), the thickness can be varied, and/or other line properties can be varied to represent data calculated by system 10 based on the recorded OCT image data or other data.

In some embodiments, one or more overlays are displayed relative to OCT image data that is being displayed via GUI 353, such as data displayed in display areas 3512a,b. In some embodiments, these one or more overlays represent segmentation data determined by an algorithm of system 10 (e.g. algorithm 1015), for example one or more overlays representing lumen segmentation, side-branch segmentation, and/or device segmentation.

Images and/or other data displayed to the operator via GUI 353 can be used to aid and/or guide the operator (e.g. a clinician) to perform a cardiac, neurological, and/or other interventional procedure, as described herein. System 10 can be configured to calculate various data metrics (e.g. via algorithms 315, 415, and/or 1015 described herein) which can be displayed to aid the operator. For example, prior to an interventional procedure (e.g. a stenting procedure), system 10 can calculate an “ideal lumen profile” (e.g. an approximation by system 10 of the lumen of a vessel segment, if no disease were present), such as by identifying diseased segments of a blood vessel, and estimating the healthy lumen profile from data collected proximal to and/or distal to the diseased segment, and/or by measuring the degree of tapering of identified side-branches (e.g. using a “branch law”, such as Murray's law). In some embodiments, after a stenting procedure system 10 can calculate an “ideal stent expansion”, for example an optimized or otherwise desirable stent expansion relative to an ideal lumen profile calculated prior to the stenting procedure. In some embodiments, an ideal stent expansion can be determined using similar processes to those used in determining an ideal lumen profile. In some embodiments, system 10 compares pre-intervention image data to post-intervention image data to identify and adjust for any changes in the appearance, diameter, and/or other characteristics of the vessel (e.g. the ostia of side-branches of the vessel) that may have been caused by the intervention (e.g. angioplasty and/or stent implantation). Such changes in vessel characteristics may alter the ideal lumen profile calculated pre-intervention and/or post-intervention. System 10 can be configured to adjust for discrepancies between pre-intervention and post-intervention ideal lumen profiles by adjusting the post-intervention data based on side-branch diameters calculated using the pre-intervention data. By adjusting the side-branch diameters calculated from post-intervention data to match the relative diameters calculated from pre-intervention data, more accurate calculation and/or comparison between the pre-intervention and post-intervention data can be performed by system 10.

Referring additionally to FIGS. 3A-3C, additional embodiments of a graphical user interface for displaying image data and guiding vascular intervention are illustrated, consistent with the present inventive concepts. The embodiments of GUI 353 shown in FIGS. 3A-3C can comprise similar workspace and/or display areas to those described in reference to FIG. 3 herein arranged similarly and/or in a different layout on GUI 353. The layout of the various workspace and/or display areas shown can be arranged to optimize the workflow intended for the user when the various embodiments of GUI 353 are displayed.

FIG. 3A shows an example of GUI 353 displayed to the user to enable the user to review the native state of an imaged vessel (e.g. the pre-intervention state). In some embodiments, OCT image data of a vessel (e.g. a coronary and/or a neurovascular vessel) is collected and analyzed by system 10 to simulate blood flow and/or to estimate the pressure drops through one or more lesions present within the vessel (e.g. FFR values). In some embodiments, simulations are performed using quasi-2D, 2D, and/or 3D models of the imaged vessel generated by system 10. These models can be based on OCT image data alone and/or a combination of OCT image data and other image data (e.g. angiographic image data). These models can include data correlating to the vessel lumen, side-branches, vessel wall characteristics, such as the presence of plaque along the vessel wall, and/or other characteristics of the imaged vessel.

As shown in FIG. 3A, FFR values can be displayed over 2D and/or 3D representations of OCT and/or angiographic image data. In some embodiments, pressure drop values and/or blood flow values are displayed. In some embodiments, FFR values are displayed as a graph, for example a graph where graphical properties of the graph (e.g. the color) are varied to highlight data of particular importance (e.g. areas of higher pressure drops). FFR data can be displayed as measured values and/or as gradients. In some embodiments, data is displayed graphically (e.g. a visual representation of numeric data), for example as shown on the right of FIG. 3A, FFR data can be displayed as dots relative to a 2D display of the lumen profile. In some embodiments, differential FFR values (e.g. comparing pre-intervention and post-intervention FFR values) can be calculated and displayed, for example as measured values and/or as gradients.

FIG. 3B shows an example of GUI 353 displayed to the user to enable simulated stenting (or other treatment) of an imaged vessel. System 10 can be configured to simulate and predict the outcome of performing a treatment procedure on an imaged vessel. For example, the user can input the desired parameters of an intervention (e.g. stenting of the imaged vessel), and system 10 can project the outcome of the treatment based on the input parameters and display the predicted results to the user. In some embodiments, system 10 is configured to analyze the image data (e.g. via algorithms 315, 415, and/or 1015 described herein) and suggest parameters of an intervention (e.g. to suggest where the imaged vessel should be stented). Treatment parameters can be selected from the group consisting of: treatment location (e.g. placement of a stent); device length; device diameter; the number of devices to be implanted; other device properties; and combinations of these. In some embodiments, system 10 is configured to perform multiple simulations to determine the “best” treatment strategy. For example, system 10 can automatically (e.g. using an AI algorithm) iterate various treatment options to identify the best option, and/or can run simulations as initiated by the user based on parameters varied by the user via GUI 353. GUI 353 can provide tools for the user to manipulate the placement and/or other parameters of a virtual stent, such that system 10 can predict the outcome of the treatment based on the user's placement of the virtual stent.

In some embodiments, GUI 353 can display “virtual” measurements (e.g. CFD, FFR, or other flow measurements) based on the predicted outcome of the planned treatment. For example, GUI 353 can display “pre” and predicted “post” treatment values, and/or delta values, for example ΔFFR values. System 10 can be configured to predict luminal gain and/or stent expansion based on the virtual stenting. In some embodiments, system 10 can provide suggestions for the preparation of an imaged vessel for a treatment procedure (e.g. in the case of calcified plaques).

FIG. 3C shows an example of GUI 353 displayed to the user to enable the user to review post-intervention image data (e.g. image data collected by system 10 after a stent has been implanted into a previously imaged vessel). Post-intervention image data can be collected part way through an interventional procedure (e.g. after one or more stents have been implanted, while more stents are still to be implanted) and/or at the end of an interventional procedure (e.g. after all planned stents have been implanted). System 10 can be configured to analyze post-intervention image data to determine the effect of the treatment performed. In some embodiments, the information displayed post-intervention can inform the user if additional treatment should be performed. System 10 can be configured to calculate CFD and/or FFR values based on the post-intervention image data. This information can help the user to determine if a pressure drop is present within a stented segment of the vessel and/or caused by stenosis outside of the stented segment. In some embodiments, system 10 can analyze and/or display information relating to the implantation of one or more stents, for example if the stent was properly expanded. In some embodiments, GUI 353 can display a virtual representation of an imaged stent relative to the image data (e.g. relative to angiographic data, cross-sectional OCT data, and/or a representation of the lumen profile).

In some embodiments, as shown in FIG. 3, pre- and post-intervention image data can be displayed to the user in a side-by-side arrangement. GUI 353 can display various metrics calculated by system 10 that quantify changes to the imaged vessel following treatment. For example, FFR gain can be quantified (e.g. ΔFFR) comparing pre- and post-intervention FFR values. Additionally or alternatively, FFR gain per length (mm) of stent can be quantified by system 10. In some embodiments, stent expansion and/or volume of malposition can be quantified and displayed to the user.

Referring now to FIGS. 4A-4D, anatomic views of a vessel showing various levels of atherosclerosis are illustrated, consistent with the present inventive concepts. In some embodiments, system 10 is configured to analyze image data (e.g. intravascular (IV) image data collected by system 10 as described herein) to estimate the pressure drop within a blood vessel using computational fluid dynamics (CFD) techniques. Boundary equations for CFD models can require the identification of a proximal frame of reference that is used by system 10 to quantify the proximal vessel diameter, diameter D_{P} shown. The proximal frame of reference can typically be identified in an image data set as a non-diseased proximal frame (e.g. a frame of image data where the imaged vessel is free of disease). FIG. 4A shows an imaged vessel free of disease. However, in many clinical cases, the main vessel shows disease (e.g. atherosclerosis) and the identified diameter does not represent the “true vessel size” (e.g. the healthy vessel diameter D_{P} shown in FIG. 4B would be underestimated, due to a vessel negative remodeling).

In some embodiments, system 10 is configured to analyze intravascular image data to determine the external elastic laminae (EEL), diameter D_{P-ELL} shown, however, due to the uncertainty of positive and/or negative remodeling in presence of atherosclerotic disease, D_{P-EEL} may often overestimate the true vessel size.

In some embodiments, system 10 is configured to use one or more vessel scaling laws to estimate the true vessel size (e.g. to determine a more accurate estimate than is provided by diameter D_{P-ELL}). Image data (e.g. OCT images captured by system 10) can be used to find a vessel segment that is not showing atherosclerotic disease (e.g. plaque), for example the segment with diameter D_{Hseg} shown, and use information relative to that segment to subsequently estimate the true vessel lumen of adjacent segments. Algorithm 1015 of system 10 (e.g. a machine learning or other AI algorithm) can be configured to automatically identify vessel segments that are free of disease. In some embodiments, algorithm 1015 can be biased toward preferentially identifying presence of disease. Alternatively, algorithm 1015 can be biased toward preferentially identifying lack of disease being present.

In some embodiments, system 10 (e.g. via algorithm 1015) can be configured to estimate diameter D_{P} based on diameter D_{Hseg} of a healthy segment of the vessel, as well as other data available to system 10 (e.g. data calculated by and/or imported into system 10). For example, system 10 can use diameter D_{SB1} of side-branch #1, shown, as well as diameter D_{Hseg} to accurately estimate D_{P}. System 10 can implement a variety of different scaling laws, for example the scaling law based on the use of 7/3 power:

$${D}_{P}^{7 / 3} = {D}_{Hseg}^{7 / 3} + {D}_{SB 1}^{7 / 3}$$

In certain scenarios, however, diffuse atherosclerotic disease can affect a longer portion of the imaged vessel, as well as the side-branches of the imaged vessel. For example, as shown in FIG. 4C, both diameters D_{seg1} and D_{SB3} do not represent their respective true diameters due to the presence of plaque in those portions of the vessels. The ostium of side-branches are often also affected in vessels showing diffuse disease.

In some embodiments, system 10 can analyze image data recorded non-invasively, for example X-ray and/or fluoroscopic image data to determine one or more of the diameters illustrated (e.g. diameter D_{SB3}). In some embodiments, image data collected from outside of the main vessel (e.g. fluoroscopic image data) can better visualize a side-branch along its entire length (e.g. better than intravascular imaging modalities described herein). In some embodiments, system 10 is configured to combine and register non-invasive image data with intravascular image data, and use the combined data to calculate one or more vessel diameters.

Alternatively, system 10 can be configured to calculate one or more vessel diameters using only intravascular image data (e.g. OCT image data collected by system 10). In some embodiments, one or more scaling laws can be applied by system 10 to multiple diameters to optimize the final estimation of all diameters and reduce errors. For example:

$${D}_{P}^{7 / 3} = {D}_{SB 1}^{7 / 3} + {D}_{seg 1}^{7 / 3}$$

$${D}_{seg 1}^{7 / 3} = {D}_{SB2}^{7 / 3} + {D}_{seg 2}^{7 / 3}$$

$${D}_{seg 2}^{7 / 3} = {D}_{SB 3}^{7 / 3} + {D}_{D}^{7 / 3}$$

can be iterated in an “optimization loop” in different ways using various mathematical techniques to reduce the discrepancy between the estimated diameters calculated by system 10.

Intravascular image data can provide information on plaque distribution that can be used by system 10 to assign weights (e.g. confidence labels) for the optimization process. For example, diameter D_{D} (diameter of the distal reference frame shown in FIG. 4C) represents a vessel segment not showing atherosclerotic disease and as such, D_{D}, D_{SB1} and D_{SB2} can be labelled with “high confidence” for the optimization process, whereas D_{seg1}, D_{seg2} and D_{SB3} are labelled with “low confidence”, (e.g. labelled by an algorithm of system 10).

FIG. 4D illustrates diffuse disease in a vessel and its side-branches. In some cases, if diffuse disease is affecting almost the totality of the intravascular image data, it may not be possible to reliably estimate the “true vessel size” from IV imaging alone. In some embodiments, system 10 can identify this scenario based on automated identification of plaques and can alert the user that a specific image data set may not be used for a reliable CFD pressure-drop calculation. Additionally or alternatively, one or more machine learning and/or other image processing methodologies can be used to automatically assess image quality, and in case of a low image quality image data (e.g. OCT image data) acquisition, a similar alert can be displayed to the user.

Referring now to FIG. 5, a method of procedure planning based on data collected and/or analyzed by the system is illustrated, consistent with the present inventive concepts. Method 1000 can be performed using the various devices of system 10 described herein. In Step 1010, image data is acquired, such as image data representing a vessel of a patient. For example, OCT image data can be recorded via a pullback procedure as described herein. Alternatively or additionally, raw and/or pre-processed data can be imported into system 10 for analysis by the system, such that the analyzed image data can be displayed to the user to assist in procedural planning.

In Steps 1020 through 1040, image processing can be performed by system 10. System 10 can comprise one or more algorithms (e.g. algorithms 315, 415, and/or 1015 described herein) for processing the image data. In some embodiments, one or more of the algorithms can comprise a bias, such as a bias as described herein.

In Step 1020, system 10 can assess the quality of the acquired image data. For example, system 10 can comprise one or more algorithms configured to assess the presence of blood in a lumen, and/or to perform catheter segmentation.

In Step 1030, system 10 can perform one or more image analyses. For example, system 10 can comprise one or more algorithms configured to perform analyses selected from the group consisting of: lumen segmentation; side-branch segmentation; vessel health analysis; and combinations of these.

In Step 1040, system 10 can calculate one or more boundary conditions based on the image data. For example, system 10 can comprise one more algorithms configured to identify reference frames of image data and/or to determine the diameters of one or more imaged side-branches.

Following the image processing performed in Steps 1020-1040, in Step 1050, system 10 can generate one or more digital models of the imaged vessel. For example, system 10 can generate a high-resolution 3D model of the imaged vessel (e.g. a model including at least a portion of one or more side-branches of the imaged vessel).

In Step 1060, system 10 can perform one or more CFD simulations to estimate various properties of the imaged vessel. For example, system 10 can perform a CFD calculation, such as described herein.

In Step 1070, various information collected and/or calculated by system 10 can be displayed to the user (e.g. via GUI 353 described herein). For example, the 3D model of the imaged vessel can be displayed to the user, along with CFD values calculated along the length of the vessel (e.g. blood flow and/or pressure drop values). System 10 can be configured to display different image data and/or system generated models simultaneously (e.g. side by side), and/or as a merged display, such as when one data type is shown overlaid on another. In some embodiments, system 10 can display both angiography image data and OCT image data.

In Step 1080, intervention planning can be performed. For example, system 10 can automatically and/or semi-automatically (e.g. via an algorithm of system 10) determine one or more interventional actions that may be performed to the imaged vessel (e.g. if disease was detected in the previous steps of method 1000). Additionally or alternatively, system 10 can be configured to provide one or more tools (e.g. via GUI 353 described herein) for the user to virtually treat the imaged vessel (e.g. to virtually insert a stent), or otherwise plan interventional actions. Method 1000 can return to step 1060 to recalculate the properties of the imaged vessel, for example while incorporating the projected outcomes of the planned interventional actions. Method 1000 can loop (e.g. at the user's discretion) through steps 1060, 1070, and 1080, such as to simulate and assess various interventional options. After an intervention plan has been determined, the user may perform the determined intervention. In some embodiments, method 1000 can be repeated after an intervention has been performed, such as to assess the outcomes of the intervention.

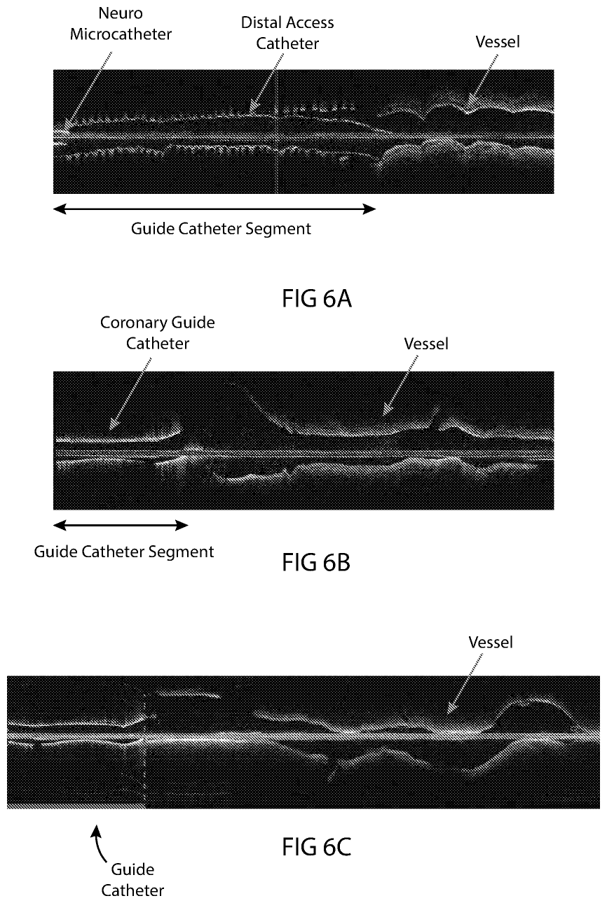

Referring now to FIGS. 6A-C, various OCT images of vessels and guide catheters are illustrated, consistent with the present inventive concepts. In some embodiments, a pullback imaging procedure ends with the optical assembly (e.g. optical assembly 115 described herein) within the proximal guide catheter (e.g. a neurovascular microcatheter, a distal access catheter, a neuro sheath, a balloon catheter, or the like, “guide catheter” herein). In these embodiments, a portion of the OCT image data represents a portion of the guide catheter (e.g. the portion of the guide catheter through which the optical assembly was retracted while imaging). For example, in some embodiments, a large portion (up to 30-40%, or more) of the total image data set can be recorded inside the guide catheter, for example as shown in FIGS. 6A and 6B. The guide catheter can either partially obscure the vessel wall and/or devices (e.g. stents) from the image, and/or can obscure the vessel from the image in its entirety. For example, depending on the construction of the guide catheter, the imaging catheter may or may not be able to image through it. For example, various guide catheters may comprise opaque plastic, transparent plastic, one or more metallic braids, and/or other features that may affect the ability of system 10 to image a vessel through the guide catheter. Guide catheters are used for a wide variety of vascular interventions, such as coronary, neurovascular, and peripheral artery interventions.

When processing an image data set (e.g. an image data set acquired by system 10), it is advantageous to identify the portion of the image data representing the vessel and/or implantable devices, and the portion of the image data collected from inside the guide catheter. In some embodiments, the guide catheter is identified and manually selected by the user. Alternatively, automated detection can be implemented (e.g. via an algorithm of system 10). Automated detection of the guide catheter can decrease the number of user interactions required to analyze an image data set.

In some embodiments, after the portion of the image data set collected from inside the guide catheter is identified (e.g. automatically identified), it can be filtered out (i.e. excluded) from further analysis. In some embodiments, identification of the guide catheter is used to provide additional information to the user, for example the user can be alerted to incomplete image acquisition if the guide catheter is not detected, and/or the user can be alerted to incorrect guide catheter placement if an implanted device (e.g. a stent) ends within the guide catheter.

In some embodiments, identifying the portion of image data comprising the guide catheter helps identify the region of interest of the image data set for further processing, and can increase the accuracy of image processing of the region of interest, for example image processing selected from the group consisting of: segmentation of intravascular devices, such as stents, flow diverters, coils, and/or other intravascular devices; segmentation of the lumen, side-branches, plaques, wall dissections, thrombus, and/or other lumen characteristics; computational fluid dynamics (CFD) calculations, such as calculations that identify pressure drops, flow characteristics, and the like; and combinations of these. Excluding image data comprising the guide catheter can provide several advantages. For example, the exclusion of data can optimize and reduce the overall image data set processing time (e.g. with less data to process subsequent image processing algorithms can be more efficient). As another example, removing this data can reduce the number of false positives (or negatives) when automatically identifying vascular and/or device features such as features selected from the group consisting of: lumen area: plaques; stenosis; device features (e.g. struts of stents, flow diverters, and the like); side-branches; intraluminal thrombus; and combinations of these. In some embodiments, system 10 can more accurately reconstruct lumen morphology in 3D (for example for fluid dynamics calculations) when the guide catheter is excluded from the image data. In some embodiments, if a large side-branch is detected in close proximity to the distal end of the guide catheter and/or blood clearance is determined (e.g. by an algorithm of system 10) to be suboptimal, a warning can be given to the user about incorrect placement of the guide catheter and/or about the suboptimal blood clearance. In some embodiments, the algorithm comprises a bias that preferentially determines incorrect placement is present (such as to sometimes allow replacement of the guide when not necessary, but avoiding scenarios in which the guide is improperly placed but not detected and left in an undesired location).

In some embodiments, the guide catheter is automatically identified using traditional signal and image processing algorithms. For example, the guide catheter can be identified by analyzing the intensity profile and/or pixel intensity of intravascular 1D, 2D, and 3D images and A-scan lines. In some embodiments, image data can be analyzed using one or more pattern recognition algorithms and/or geometrical transformations such as a Hough transform and/or image cross-correlation. Alternatively or additionally, the guide catheter can be identified using Artificial Intelligence (AI) methodologies as described herein, such as methodologies selected from the group consisting of: a 2D convolutional encoder network; a Dual-Domain encoder network; other types of neural networks, including various different types of convolutional networks; a combination of traditional signal and image processing algorithms with AI algorithms; and combinations of these. In some embodiments, image processing algorithms of system 10 (e.g. algorithms 315, 415, and/or 1015 described herein) are used to pre-process and/or post-process image data and/or results determined using various artificial intelligence algorithms.

In some embodiments, an algorithm of system 10 is configured to identify a guide catheter in one or more 2D cross-sectional OCT images (e.g. B-mode images) in polar and/or cartesian format, and/or in one or more longitudinal view (e.g. l-mode) images. The 2D method returns a probability measure that a given slice (e.g. a 2D OCT cross sectional image) contains a guide catheter or not. This method can be applied iteratively and/or using a binary search pattern to find the start (e.g. first occurrence), and/or the end (e.g. the last occurrence) of the guide catheter in the image data set. For example, an algorithm of system 10 can comprise a ‘DD2Net Full Fusion Classifier architecture’ that takes advantage of both polar and cartesian information to determine the probability of the presence of a guide catheter in a frame of an image data set. An identified guide catheter can be displayed to the user using various techniques, for example on 2D cross-sectional OCT images, or on 2D longitudinal view (l-view), such as shown in FIG. 6C, and/or with 3D visualization techniques (such as are described herein).

Referring now to FIGS. 7A-7D, images to be displayed to a user representing OCT image data and image quality are illustrated, consistent with the present inventive concepts. The images shown in FIGS. 7A-7D can be displayed to the user via a graphical user interface, such as GUI 353 described herein. FIG. 7A shows OCT image data displayed as a longitudinal view, as well as a representation of profile of the imaged lumen. In some embodiments, an image data quality indicator can be displayed relative to the displayed OCT image data, indicator 3523 shown. FIGS. 7B-7D show OCT image data displayed as cross-sectional views. The arrows shown indicate the relation between FIGS. 7B-7D and the longitudinal data displayed in FIG. 7A. In some embodiments, an image data quality indicator can be displayed relative to the cross-sectional OCT image data, indicator 3524 shown. Indicators 3523 and/or 3524 can indicate image data quality, for example image data quality as assessed by system 10, as described herein. In some embodiments, portions of the image determined to have poor image data quality are highlighted to warn the user, such as is shown (e.g. highlighted with a color such as red). Alternatively or additionally, portions of the image with good image data quality can be highlighted. In some embodiments, a scale can be displayed, such as a scale configured to indicate the values of the information displayed by indicators 3523 and/or 3524.

In some embodiments, system 10 is configured to automatically assess the quality of image data collected by the system. The quality of the acquired data (e.g. OCT image data collected by system 10) depends on many factors related to imaging acquisition. For example, low quality image data can be observed when OCT data acquisition happens in conditions of incomplete blood clearance. In some embodiments, system 10 comprises an automated machine learning based algorithm configured to automatically classify images based on their quality and probability to contain blood. System 10 can be configured to adjust one or more image processing procedures described herein based on this classification, such as to prevent or at least limit incorrect segmentation and/or automated analysis of low-quality frames or image data. Additionally or alternatively, system 10 can provide indications to the user about potential low-quality acquisition for an improved clinical workflow for OCT image data analysis.

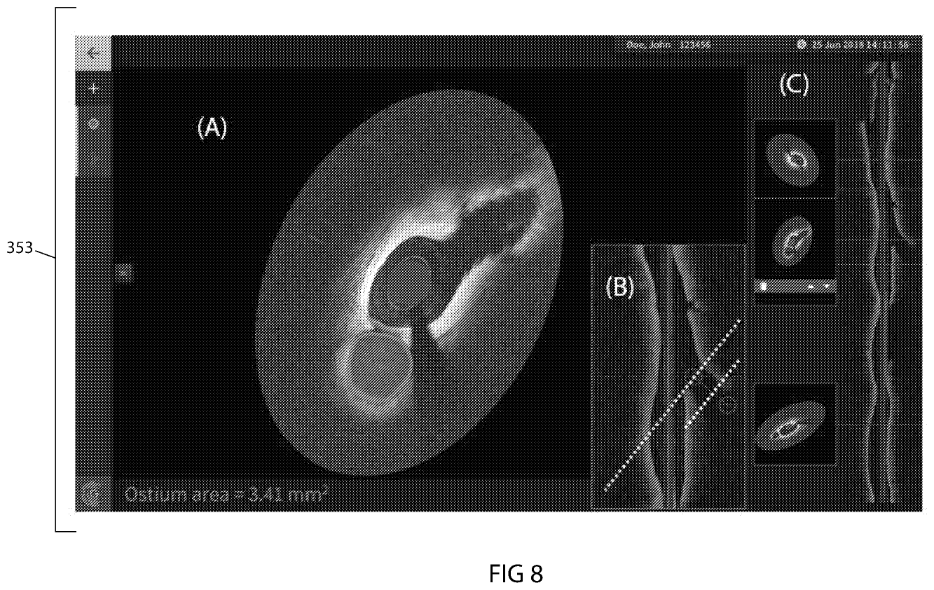

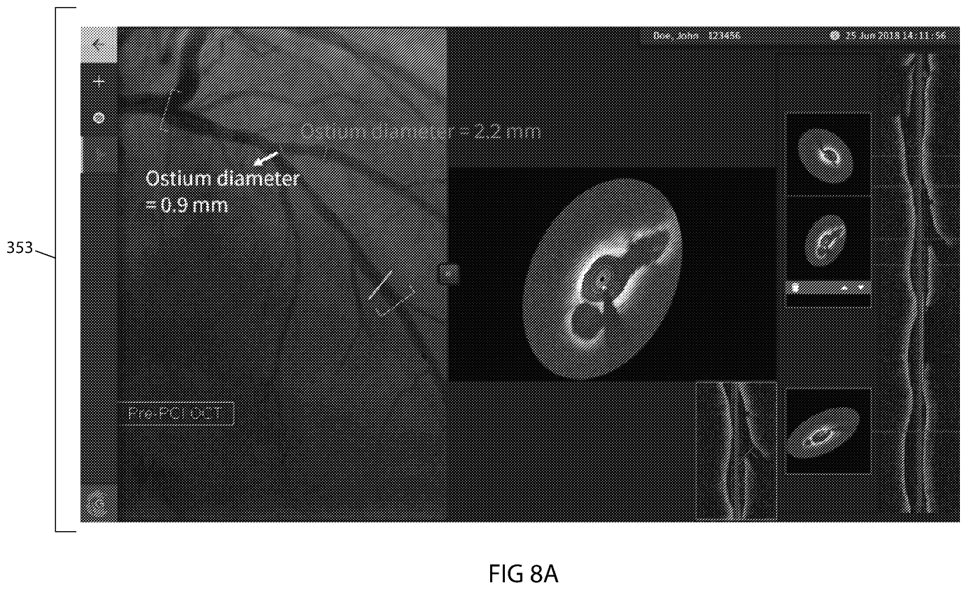

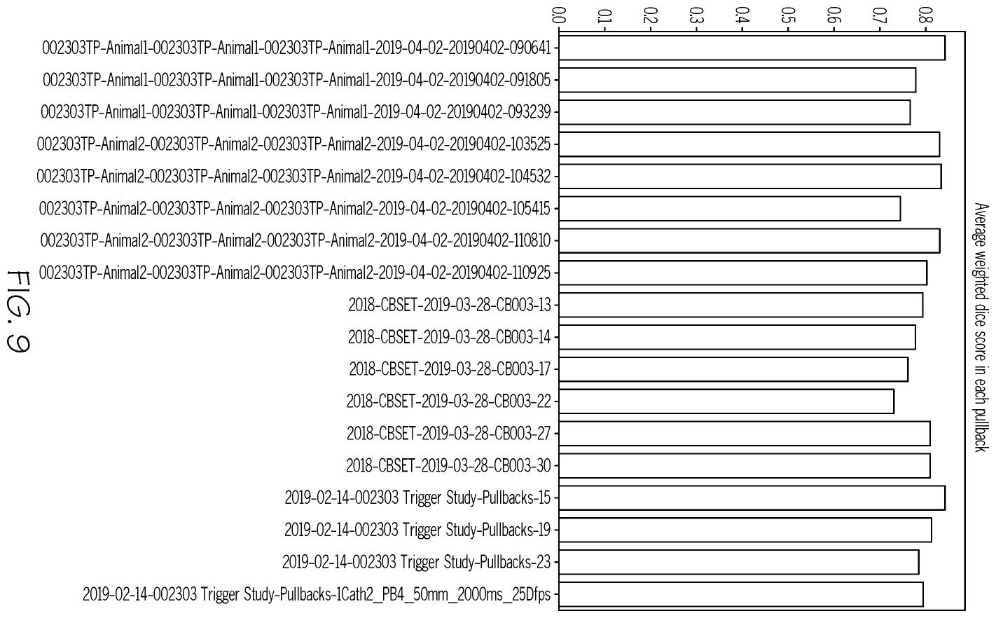

Referring now to FIG. 8, an embodiment of a graphical user interface for displaying image data and allowing a user to review information determined by the system based off of the image data is illustrated, consistent with the present inventive concepts. GUI 353 of FIG. 8 can be similar to GUI 353 described herein. GUI 353 can be configured to enable the user to review, approve, and/or edit the results of one or more image processing steps that have been performed by system 10 (e.g. performed by an algorithm of system 10 as described herein). For example, system 10 can be configured to identify any side-branches of an imaged vessel, and/or to determine one or more properties of the identified side-branches, such as the diameter of the branches. GUI 353 can display the calculated information for user review. For example, workspace B displays a line indicating the calculated side-branch angulation and cut plane relative to a longitudinal display of the OCT image data. Workspace A includes an indicator showing the perimeter side-branch ostium projection relative to a cross-sectional display of the OCT image data. Workspace C indicates the various side-branches detected relative to another longitudinal display of the OCT image data.

In some embodiments, GUI 353 enables the user to review the various automatically identified side-branches by selecting each branch from workspace C, and review the data displayed in workspaces A and/or B. In some embodiments, if the user agrees with the displayed information relating to an automatically identified side-branch, the user can approve (or otherwise confirm) the information. In some embodiments, the user confirms the information displayed relative to a selected side-branch with a single input (e.g. a single “click”). GUI 353 can also enable the user to edit the displayed information (e.g. to override the automatically generated information). For example, in workspace A, the user can edit the presented image of the side-branch ostium perimeter previously estimated by system 10. Additionally or alternatively, in workspace B the user can modify the angulation and/or the cut plane. In some embodiments, after one or more user modifications to the data, system 10 calculates and/or recalculates values based on the edited data. For example, if the user adjusts the side-branch ostium perimeter, system 10 can calculate the area based off of the user modified perimeter. Additionally, if the user adjusts the angulation and/or the cut plane, system 10 can recalculate the ostium perimeter based off of the user modified angulation. Using workspace C, the user can review all identified side-branches, and correct false positives and/or false negatives by removing and/or adding side-branch identifications, respectively. In some embodiments, system 10 is configured to only display side-branches that have been identified to have a diameter above a threshold, for example a diameter greater than 1 mm.

Referring additionally to FIG. 8A, another embodiment of a graphical user interface for displaying image data and allowing a user to review information determined by the system based off of the image data is illustrated, consistent with the present inventive concepts. In FIG. 8A, angiography image data is displayed alongside workspaces A, B, and C shown in FIG. 8. System 10 can be configured to register OCT image data to angiography image data, such that features (e.g. side-branches) identified by analysis of the OCT image data can be identified in the angiography image. In some embodiments, system 10 can be configured (e.g. via algorithms 315, 415, and/or 1015 described herein) to analyze both OCT image data and angiography image data to identify features of the OCT imaged vessel. Analysis of different types of image data (e.g. manual analysis by a user and/or automatic analysis performed by system 10) can produce more accurate results compared to analysis of a single image data type. For example, if a side-branch ostium is heavily diseased, such that it is difficult to determine the diameter via OCT image data, angiography image data can allow for a more accurate estimate of the side-branch diameter (e.g. by analyzing a portion of the side-branch otherwise not visible in the OCT image data). In some embodiments, the information calculated, displayed to, and/or confirmed by the user as described herein can be utilized by system 10 to perform subsequent analyses, for example to perform CFD calculations as described herein (e.g. calculations based off of the identified vessel and/or side-branch diameters).

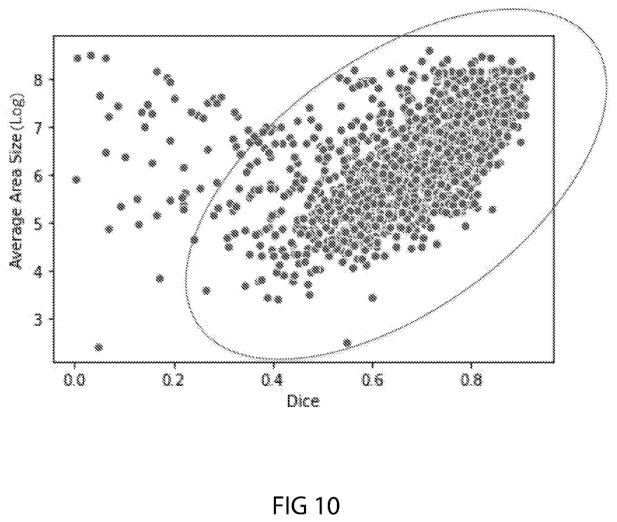

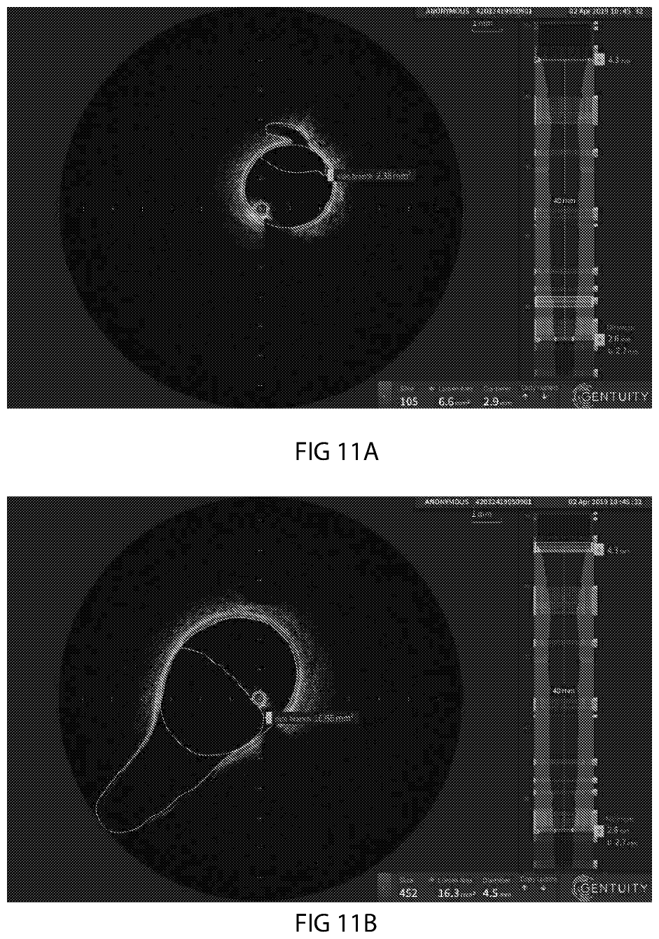

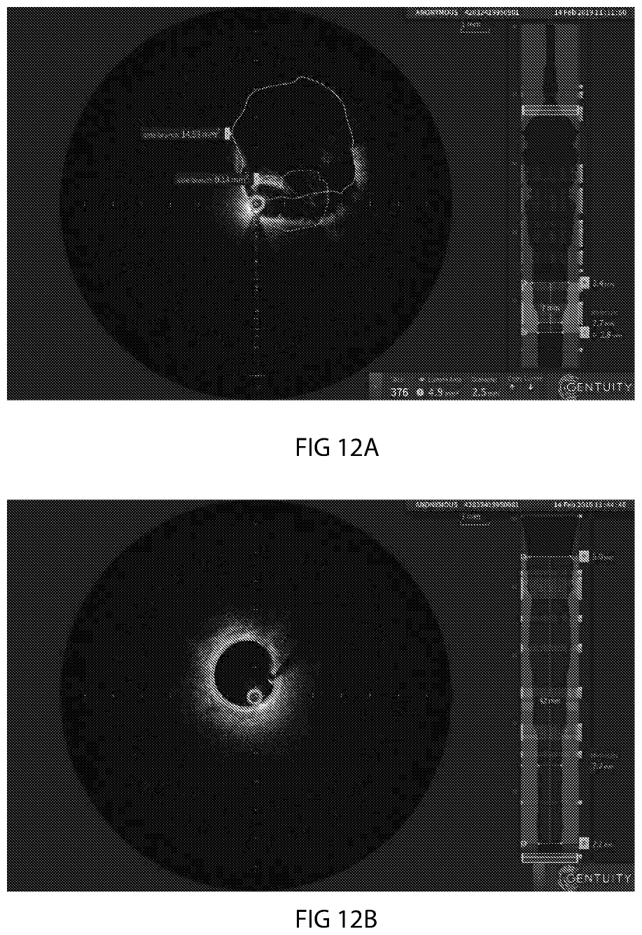

Referring now to FIGS. 9-12B, various representations of data collected by the applicant are illustrated, consistent with the present inventive concepts. In some embodiments, system 10 comprises an AI algorithm, such as algorithm 1015 described herein. Algorithm 1015 can be trained to perform side-branch segmentation (e.g. to identify one or more side-branches of an imaged vessel by analyzing image data). In some embodiments, algorithm 1015 comprises a DD2Net Full Fusion architecture. Applicant has trained and tested such an algorithm, with training data comprising image data collected from approximately 70 pullbacks, including approximately 24,000 images. Applicant evaluated the algorithm using a Weighted Dice Score across more than 1500 images that included a side-branch. A sample of the results is shown in FIG. 9. Applicant testing showed an average dice score of 0.81. FIG. 10 shows a correlation between dice score and the average area of the side-branch detected, where larger areas generally resulted in a higher dice score (e.g. a better segmentation performed by the algorithm). FIGS. 11A and 11B illustrate segmentation of relatively small and relatively large side-branches, respectively. In some embodiments, algorithm 1015 is biased toward more accurately identifying larger side-branches at the cost of misidentifying smaller side-branches, as larger side-branches have a greater effect on CFD or flow calculations based off of the segmented data. In some embodiments, algorithm 1015 comprises a threshold for identifying side-branches, for example a size threshold where side-branches smaller than the threshold are ignored by algorithm 1015. For example, algorithm 1015 can be configured to ignore side-branches with a diameter smaller than 2 mm, such as smaller than 1 mm, such as smaller than 0.5 mm. Outliers in the data shown in FIG. 10 are generally caused by poor image quality, for example the image shown in FIG. 12A illustrates a poorly identified side-branch in an image with very poor quality. FIG. 12B illustrates a high-quality image for reference. In some embodiments, an algorithm of system 10 (e.g. algorithm 1015) is configured to identify poor image quality, and to alert the user that the results of further processing of that image (e.g. segmentation results) may have a low confidence value, as described herein.

System 10 can be configured to generate a 3D model of one or more imaged vessels including one or more side-branches of that vessel. In some embodiments, the model is generated at least in part based off of segmentation (e.g. side-branch segmentation) performed by algorithm 1015 as described herein. System 10 can be configured to generate the model using various surface generation algorithms, for example a “marching cubes” algorithm. In some embodiments, system 10 can include one or more software toolkits for modeling tissue, for example the Vascular Modelling Toolkit (VMTK).

Referring now to FIG. 13, OCT image data showing the results of poor catheter purging and good catheter purging is illustrated, consistent with the present inventive concepts. FIG. 13 shows a side-by-side comparison of reduced quality images (images on the left portion of FIG. 13) due to incomplete catheter purging, and good quality images (images on the right portion of FIG. 13) due to a complete purge having been performed. Speckles representing presence of blood between the two catheter sheaths can be seen in the left catheter image magnification, whereas black space between the two sheaths in the right image denotes full purge of blood (i.e. presence of flush media between the sheaths).

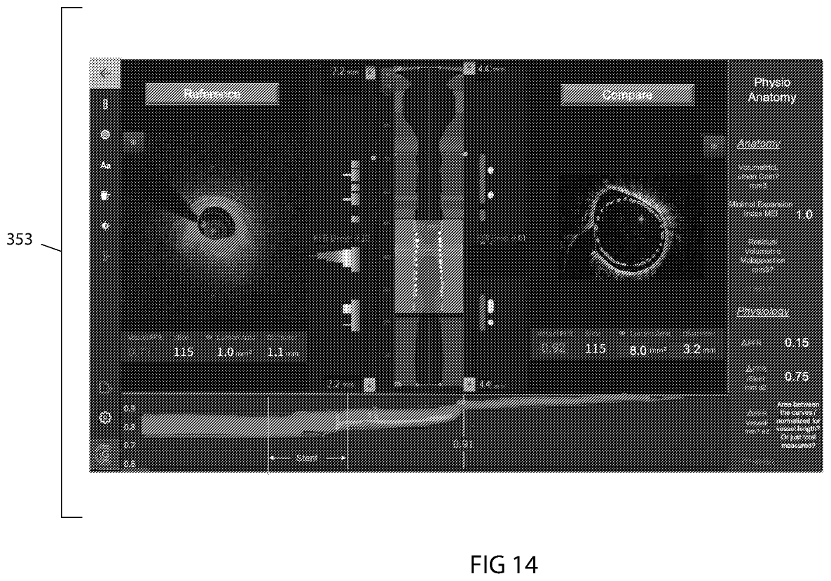

Referring now to FIG. 14, another embodiment of a graphical user interface for displaying image data and guiding vascular intervention is illustrated, consistent with the present inventive concepts. GUI 353 of FIG. 14 can be similar to GUI 353 described herein. In the clinical setting, assessment of anatomic and/or physiologic parameters of the patient has been demonstrated to support better physician decision making, and frequently leads to better outcomes, as well as decreased cost of care. Due to cost and complexity of the tools currently available to provide these assessments (e.g. individual tools each configured to provide a unique piece of anatomic and/or physiologic information), they are inconsistently utilized. Furthermore, when these tools are used together, they can increase procedural time, cost, complexity, and/or risk, and they do not provide the information in an integrated, readily comprehensible manner. System 10 of the present inventive concepts is configured to provide a better understanding of both anatomy and physiology of the patient, which will result in better procedural planning, enhanced safety, and improved efficacy.

GUI 353 can provide a single interface comprising multiple workspaces (e.g. workspace area 3501 and/or 3502 describe herein), where the user can select a workspace of interest and data displayed elsewhere (e.g. in other workspaces) is synched automatically to the workspace of interest (e.g. a time index and/or a location index can be adjusted in the workspace of interest and updated in other workspaces to display correlating data). In some embodiments, a pre-intervention lumen profile is displayed in an overlay fashion relative to a post-intervention lumen profile. GUI 353 can be configured such that the user can toggle a workspace between different types of image data, for example between OCT and angiography image data. In some embodiments, when the data is toggled between data types, the two data sets are synchronized. GUI 353 can be configured such that the user can toggle a workspace between similar image data types collected at different times and/or at different locations (e.g. pre and post intervention). In some embodiments, a first set of information (e.g. pre-intervention side-branch information) is displayed relative to a second set of information (e.g. post-intervention lumen profile information). GUI 353 can provide a procedural planning interface, where the clinician can perform virtual stenting, such as is described herein. GUI 353 can display a lumen profile determined by analyzing image data collected by system 10, as well as an “ideal” lumen profile, calculated by system 10, as described herein. GUI 353 can display one or more pressure curves, such as pre-intervention pressure curves calculated by system 10, and/or predicted post-intervention pressure curves calculated based on the virtual stenting (e.g. based on the length and placement of the virtual stent).

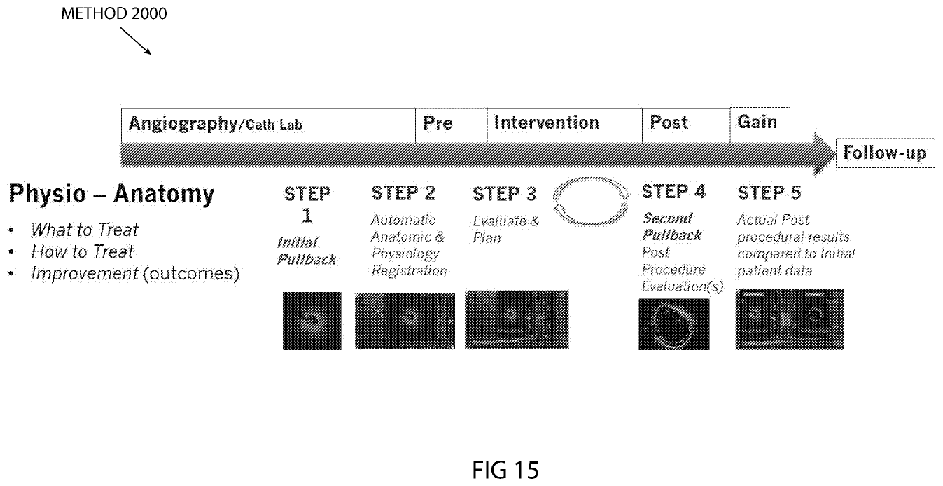

Referring now to FIG. 15, a method of treating a patient including planning and evaluating a treatment plan is illustrated, consistent with the present inventive concepts. Method 2000 can be performed using the various devices of system 10 described herein. In step 1, image data of a patient site is collected by system 10 in an initial (e.g. pre-intervention) pullback procedure. In step 2, system 10 performs anatomic and/or physiologic assessments (e.g. automatically) of the image data, as described herein. In step 3, the clinician, via GUI 353 of system 10, evaluates the assessment data provided by system 10 and plans an interventional treatment based on the data provided, and then performs the interventional treatment following the plan. In step 4, additional image data is collected by system 10 in a second pullback procedure. The image data collected in the second pullback is analyzed by system 10 and/or the clinician. In some embodiments, steps 3 and 4 are repeated, for example until a desired treatment outcome has been achieved. In step 5, post procedural results can be compared to pre-intervention data.

In step 1, an initial pullback can comprise a pullback of approximately 100 mm collected over approximately 2 seconds. System 10 can be configured to perform an initial image quality assessment, such as an assessment of lumen clearing, location of the guide catheter, and/or the identification of healthy segments of the imaged vessel (each as described herein). In some embodiments, system 10 is configured to identify any side-branches of the imaged vessel. System 10 can be configured to allow the user to review and/or edit the identified side-branches, such as is described herein.

In step 3, system 10 can be configured to model results of a virtual treatment (e.g. virtual stenting) to predict the outcome of the treatment. For example, system 10 can model the FFR gain that would be achieved from implanting a stent with optimal expansion of the stent. System 10 can provide the model based on the length and implantation location of the stent (e.g. as input by the clinician into system 10).

In step 4, a second pullback can comprise a pullback of approximately 100 mm collected over approximately 2 seconds. System 10 can be configured to perform an initial image quality assessment, such as an assessment of the imaging of the stent, location of the guide catheter, and/or the identification of healthy segments of the imaged vessel (each as described herein). System 10 can be configured to calculate actual treatment results based on the image data, and to compare those results to the modeled results calculated in step 3. In some embodiments, system 10 is configured to identify improvement opportunities, such as modifications that can be made to the implanted stent (e.g. further expansion of the stent), and/or where additional stents or other treatments could be performed. In some embodiments, the user selects one or more reference frames within the image data, such as to do a side-by-side comparison of various vessel locations pre and post intervention.

In step 5, relative metrics between pre-intervention data and post-intervention data can be displayed to the user, such as FFR gain.

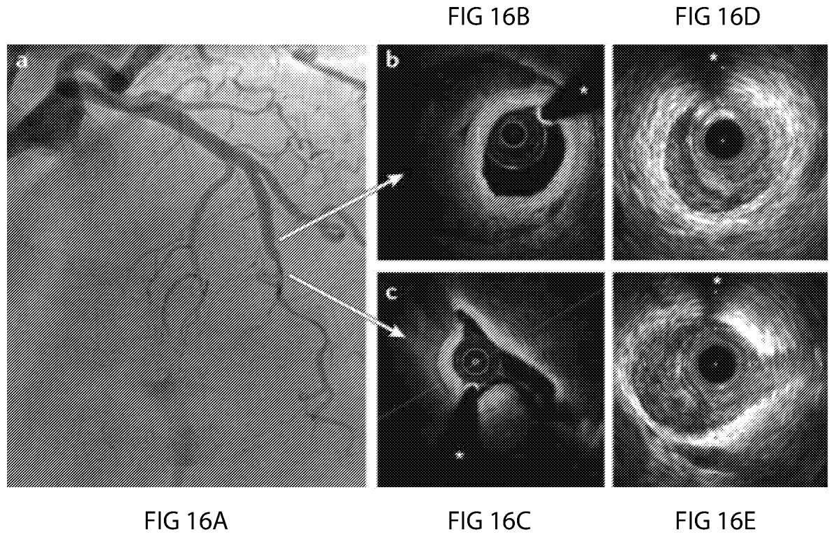

Referring now to FIGS. 16A-E, examples of various types of image data are illustrated, consistent with the present inventive concepts. FIG. 16A shows an angiography image, comprising a relatively low resolution 2D projection. FIGS. 16B and 16C show slices of OCT images recorded within a vessel shown in FIG. 16A. FIGS. 16D and 16E show similar slices of IVUS images recorded within the same vessel.

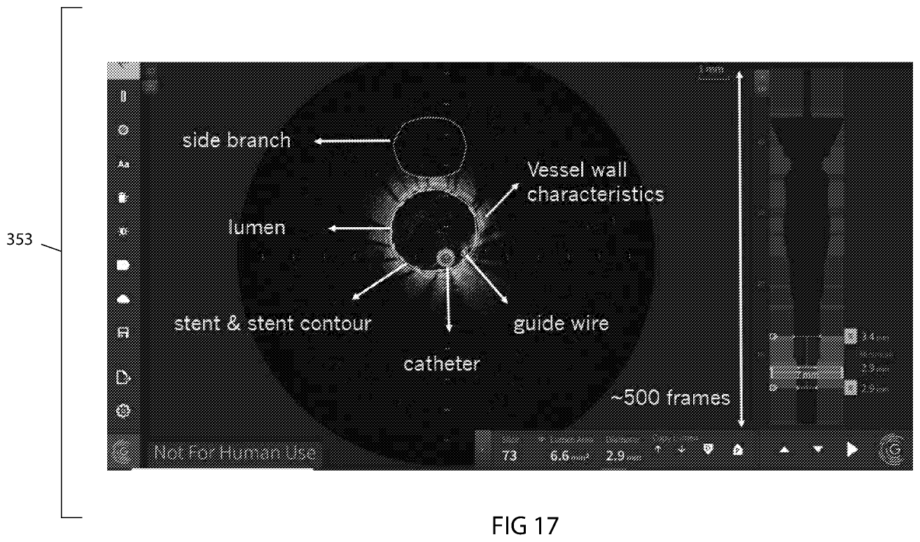

Referring now to FIG. 17, an embodiment of a graphical user interface for displaying image features automatically identified by an image processing algorithm is illustrated, consistent with the present inventive concepts. In some embodiments, algorithm 1015 is configured to analyze image data and segment one or more features identified within the data. As shown in FIG. 17, algorithms 1015 can be configured to segment one or more features selected from the group consisting of: one or more side-branches; lumen walls; stent struts; stent contour; a portion of a catheter; a portion of a guide wire; vessel wall characteristics; and combinations of these. In some embodiments, algorithm 315/415 comprises a machine learning algorithm, such as a convolutional neural network (CNN). A CNN can comprise a neural network with deep layers, and/or a neural network that applies a convolution calculation. In some embodiments, CNN algorithms are shift invariant, space invariant, and/or are sensitive to edges. In some embodiments, system 10 comprises a CNN or other machine learning algorithm that has been trained using image data collected by system 10. Training data can comprise image data sets that have been augmented to provide a balanced training set. For example, low quality images can be duplicated to create a balance between high- and low-quality images. Images comprising side-branches can be duplicated to create a balance between images containing and not containing side-branches. Images comprising stents and/or other devices can be duplicated to create a balance between images containing and not containing various devices. In some embodiments, one or more images (e.g. each image) of the training data set is randomly shifted, zoomed, and/or rotated.

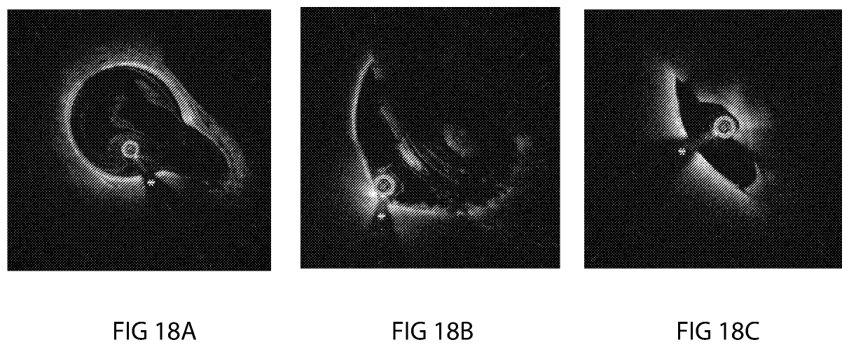

Referring now to FIGS. 18A-18C, preprocessed examples of image data with varying levels of blood in each image are illustrated, consistent with the present inventive concepts. In FIG. 18A, there is blood in the image, but the image is overall clear. In FIG. 18B, there is a significant amount of blood in the image, but enough data is clear such that a lumen profile can be inferred, such as from adjacent frames. In FIG. 18C, the image is nearly fully blocked by blood. In some embodiments, system 10 is configured to detect the presence of blood in an image, such as is described herein. In some embodiments, system 10 classifies an image as having blood or not (e.g. a binary classification relative to a threshold amount of blood). Alternatively or additionally, the image can be classified by a percentage or other metric related to the amount of blood in the image. In some embodiments, algorithm 1015 comprises a CNN configured to detect the presence of blood in a frame of image data. Algorithm 1015 can be configured to consider image data from adjacent frames. The output of the CNN can comprise a probability map (e.g. the probability of the presence of blood in each frame of image data).

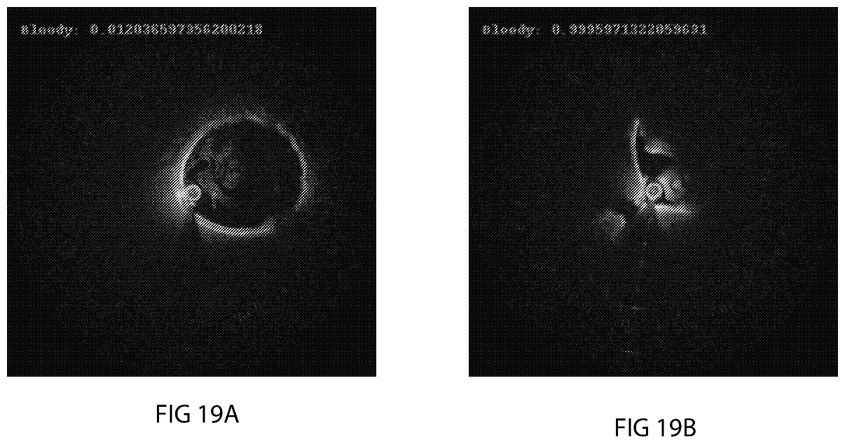

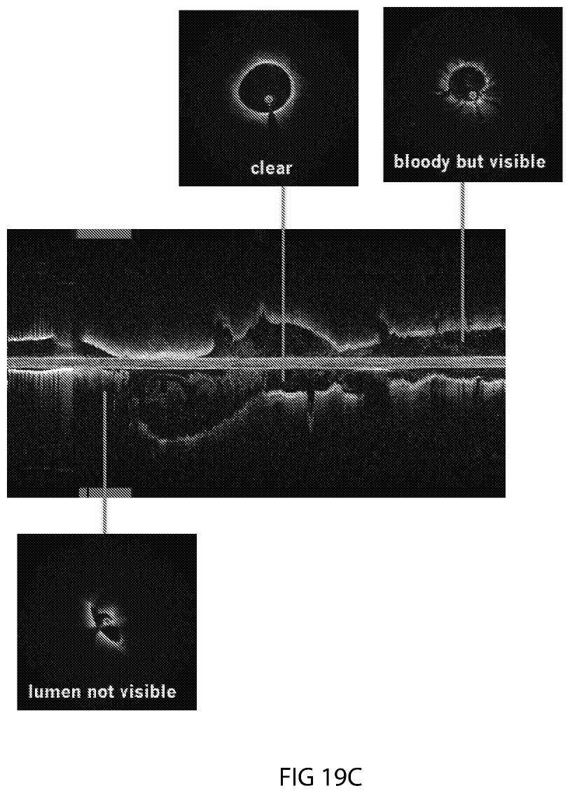

Referring additionally to FIGS. 19A-C, additional OCT images are illustrated, consistent with the present inventive concepts. FIGS. 19A and 19B show OCT image slices with a calculated probability of blood in the image displayed relative to each image. The probability shown in FIGS. 19A and 19B was calculated using algorithm 1015 described herein. FIG. 19C shows frames along a lumen gram with varying amounts of blood in each frame.

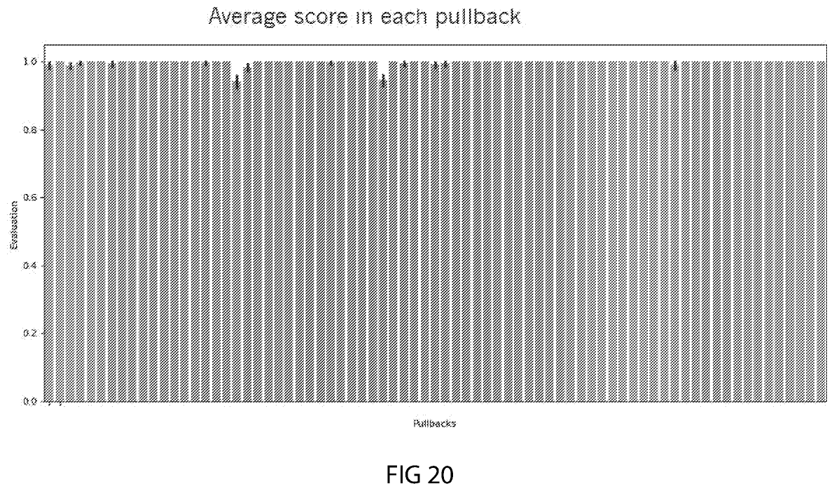

Referring now to FIG. 20, results of testing performed by the applicant are illustrated, consistent with the present inventive concepts. Applicant has trained and tested a CNN algorithm for blood detection, with a training data set comprising image data collected from 70 pullbacks. Applicant evaluated the algorithm, and found a 99.57% accuracy, with a sensitivity of 98.0% and specificity of 99.6%. The testing yielded a false positive result of 0.38% (105 images) and false negative of 0.05% (14 images).

Applicant has also trained and tested the same CNN algorithm for guide catheter detection, with a training data set comprising image data collected from 70 pullbacks. Applicant evaluated the algorithm, and found a 99.99% accuracy, with a sensitivity or 99.99%, and a specificity of 100%.

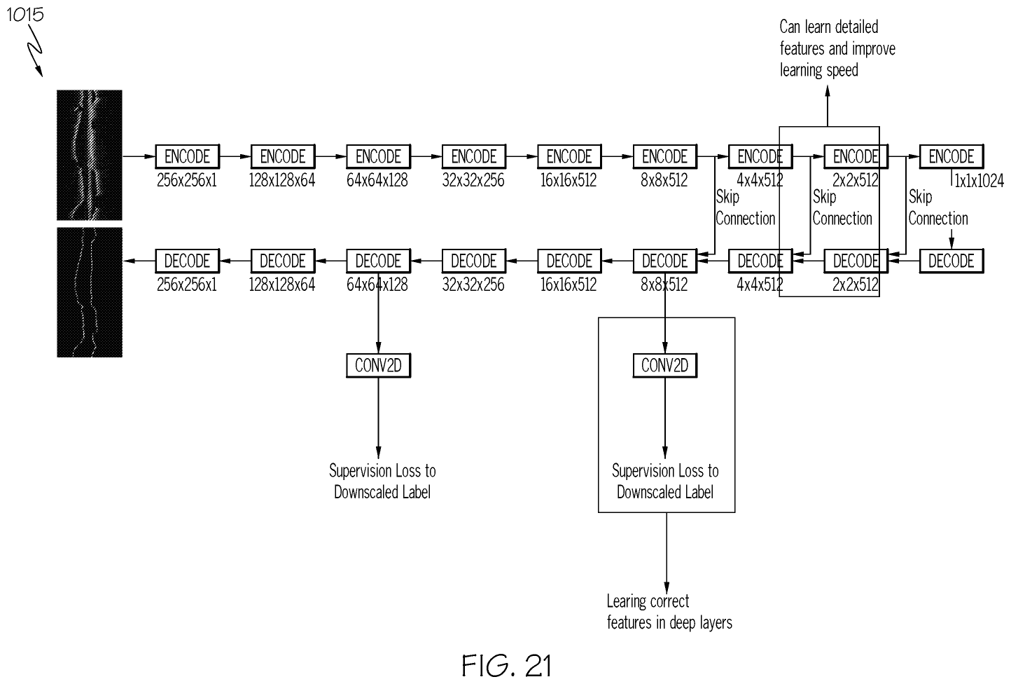

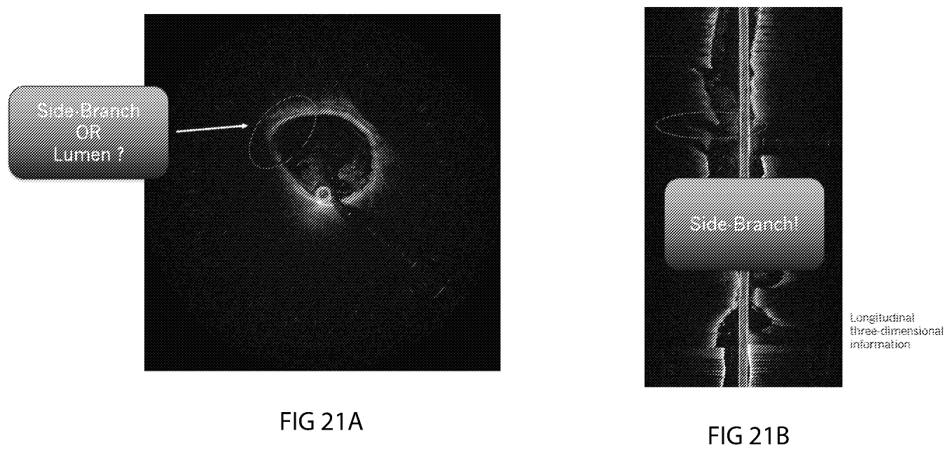

Referring now to FIG. 21, a graphical representation of a neural network is illustrated, consistent with the present inventive concepts. FIG. 21 depicts an algorithm configured as a neural network, such as algorithm 1015 described herein. In some embodiments, algorithm 1015 comprises a neural network configured to identify the boundaries of an imaged lumen (e.g. lumen segmentation) by analyzing longitudinal information (e.g. a longitudinal method), as shown in FIG. 21. Alternatively or additionally, algorithm 1015 can comprise a neural network configured to perform lumen segmentation using a polar and cartesian dual domain model for analyzing individual image slices, such as is described in reference to FIG. 2 and otherwise herein. In some embodiments, algorithm 1015 is configured to perform lumen segmentation using both a dual domain method as well as a longitudinal method. By analyzing image data using multiple methods, a more robust solution can be achieved. For example, without information interpreted from a longitudinal model, algorithm 1015 may not be able to distinguish between the main arterial lumen and the lumen of a side-branch, for example as demonstrated by FIGS. 21A and 21B.

Referring additionally to FIGS. 21A and 21B, an image frame and longitudinal image data are illustrated, respectively, consistent with the present inventive concepts. FIG. 21A shows an image frame comprising a portion that is difficult to distinguish between a wall of the lumen of the imaged vessel and a portion of a side-branch of the imaged vessel. FIG. 21B shows longitudinal image data for the same vessel, which indicates the unknown portion of the image frame in fact comprises a portion of a side-branch. By combining both domains, algorithm 1015 provides a more robust lumen segmentation.

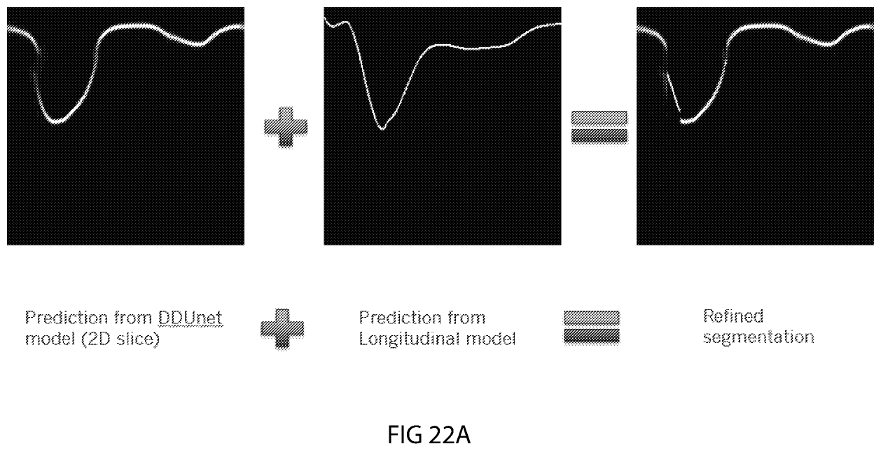

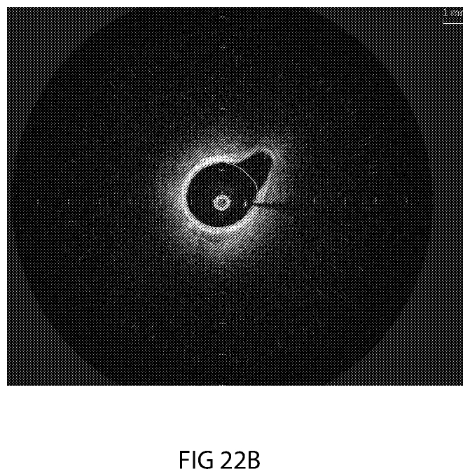

Referring additionally to FIGS. 22A and 22B, a representation of the combined method segmentation and an example of segmented image data are illustrated, respectively, consistent with the present inventive concepts. As shown in FIG. 22A, lumen segmentation performed by analyzing individual frames of image data (e.g. a 2D slice of image data) can be combined with segmentation performed by analyzing a longitudinal model of image data to produce a refined segmentation result. FIG. 22B shows a frame of image data comprising a portion of a side-branch, where the segmented lumen follows the lumen profile and not the side branch profile.

In some embodiments, algorithm 1015 (e.g. algorithm 1015 of FIG. 21 and/or FIG. 2) is configured to skip one or more layers of its neural network to perform one of multiple trained image processing applications (e.g. each module of algorithm 1015 only uses the layers of the neural network that are required to perform the segmentation).

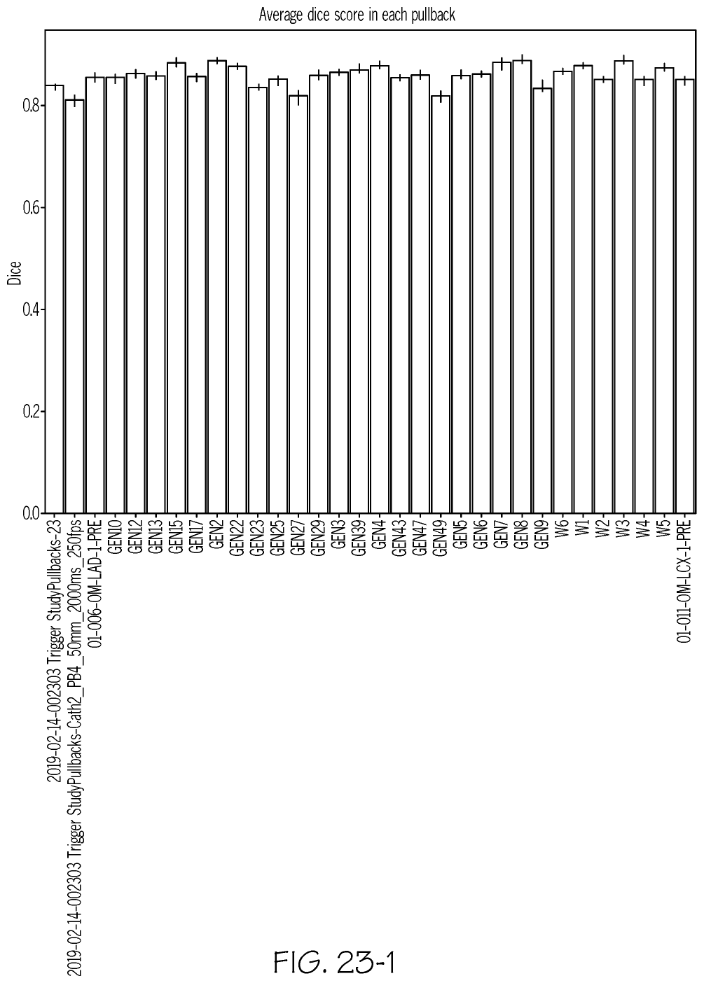

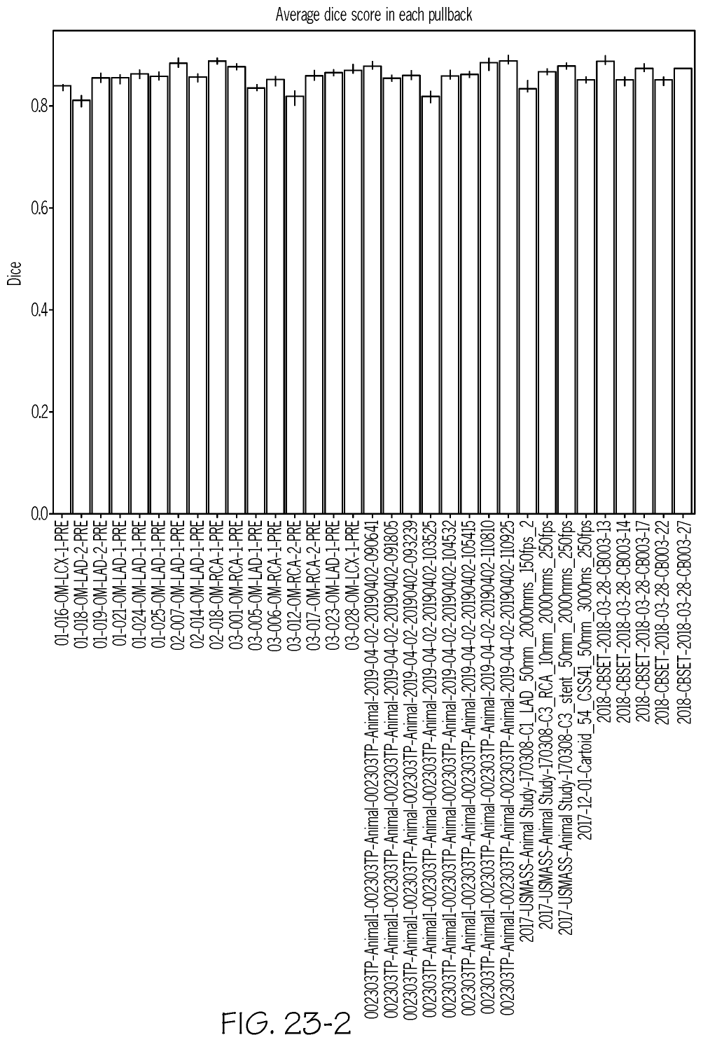

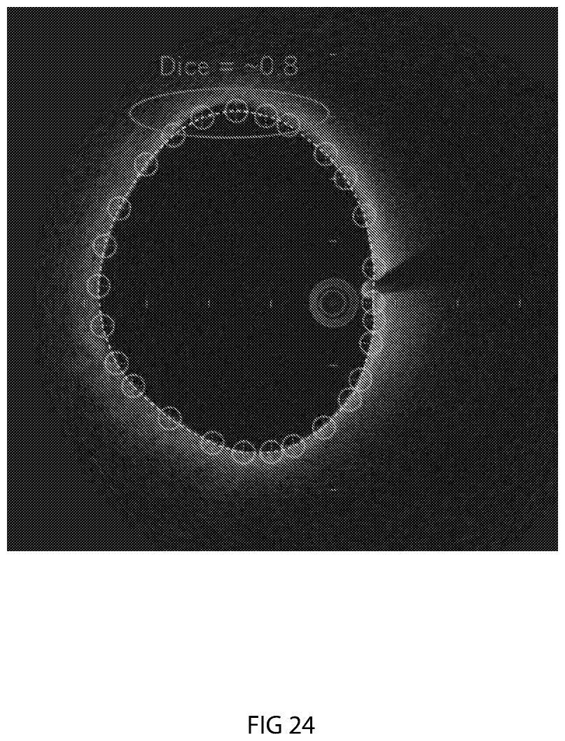

Referring now to FIGS. 23 and 24, various representations of data collected by the applicant are illustrated, consistent with the present inventive concepts. In some embodiments, system 10 comprises an AI algorithm, such as algorithm 1015 described herein. Algorithm 1015 can be trained to perform lumen segmentation, such as is described herein. Applicant has trained and tested such an algorithm, with training data comprising image data collected from 65 pullbacks. Applicant evaluated the algorithm using a Weighted Dice Score. A sample of the results are shown in FIG. 23. FIG. 24 shows an example of a segmented image with a dice score of approximately 0.8. Applicant testing showed that 90% of segmented images resulted in a dice score of greater than 0.795.

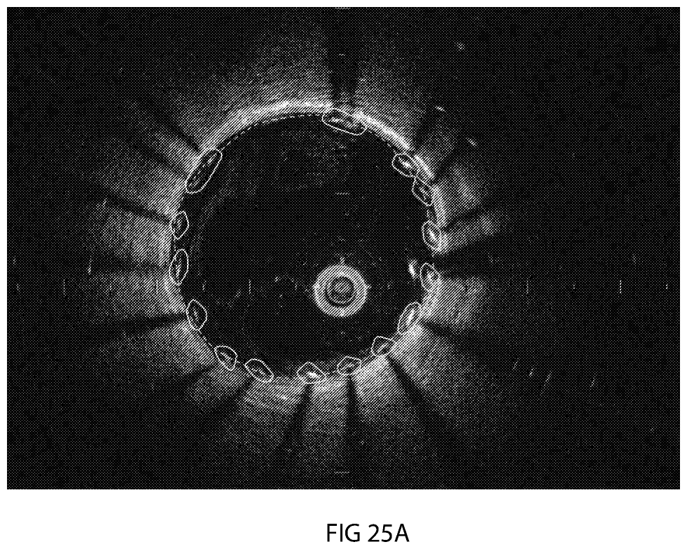

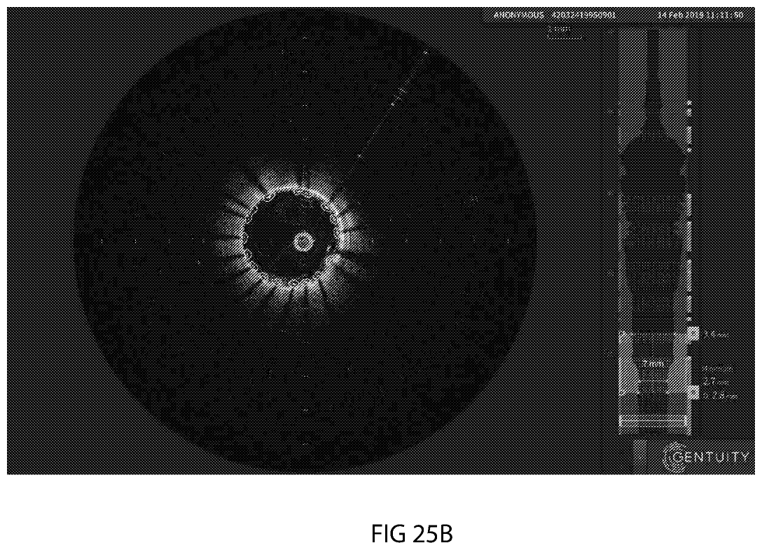

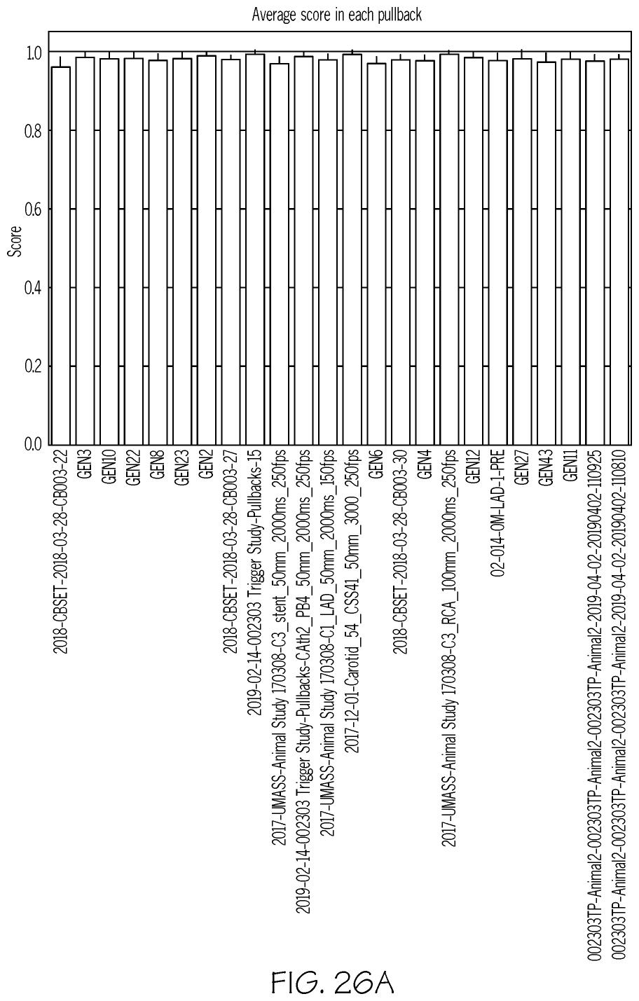

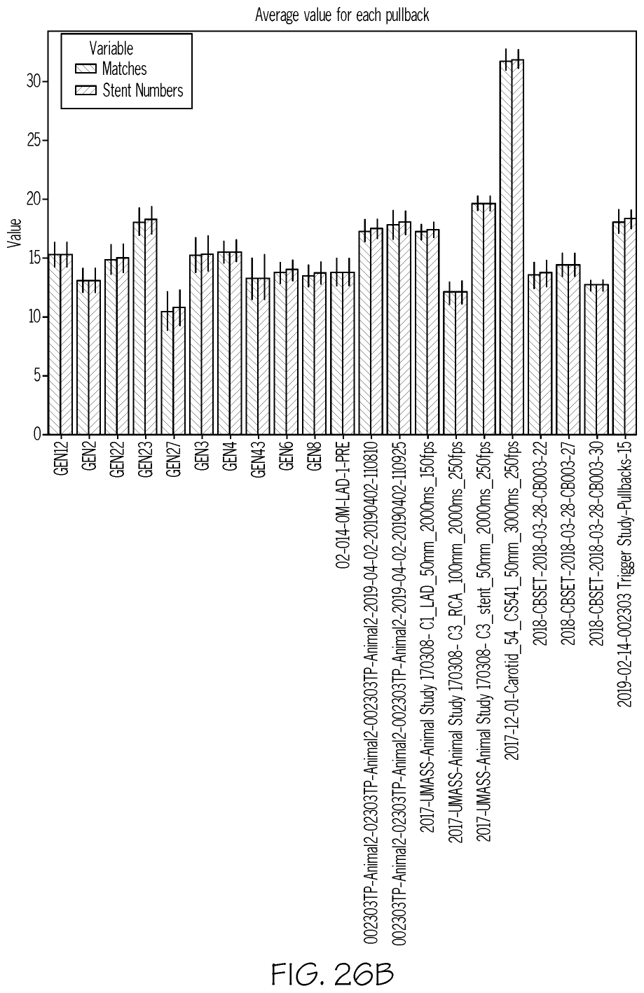

Referring now to FIGS. 25A-26B, various representations of data collected by the applicant are illustrated, consistent with the present inventive concepts. In some embodiments, system 10 comprises an AI algorithm, such as algorithm 1015 described herein. Algorithm 1015 can be trained to perform stent detection, such as to automatically quantify one or more stent features, such as stent area and/or apposition. Algorithm 1015 can be configured to depict and quantify side-branch coverage, and/or to quantify stent healing. In some embodiments, algorithm 1015 comprises a DD2Net Full Fusion architecture (e.g. similar to algorithm 1015 described in reference to FIG. 9 herein). Applicant has trained and tested such an algorithm, with training data comprising image data collected from 70 pullbacks, including approximately 24,000 images. Examples of segmented images are shown in FIGS. 25A and 25B. Applicant evaluated the stent segmentation algorithm by calculating the percentage of actual stent struts that were segmented relative to the total number of stent struts in each image frame (e.g. as determined manually and/or by other methods). Testing resulted in an average score of greater than 99.2 across 24 pullbacks. FIG. 26B shows a comparison of an average number of identified struts to actual struts for a sample of pullbacks tested. Testing showed a false positive rate of 0.46%, and a false negative rate of 0.15%.

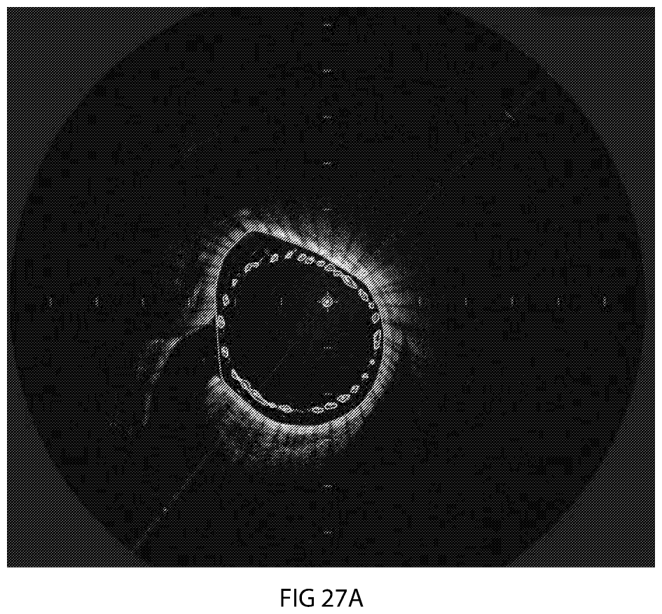

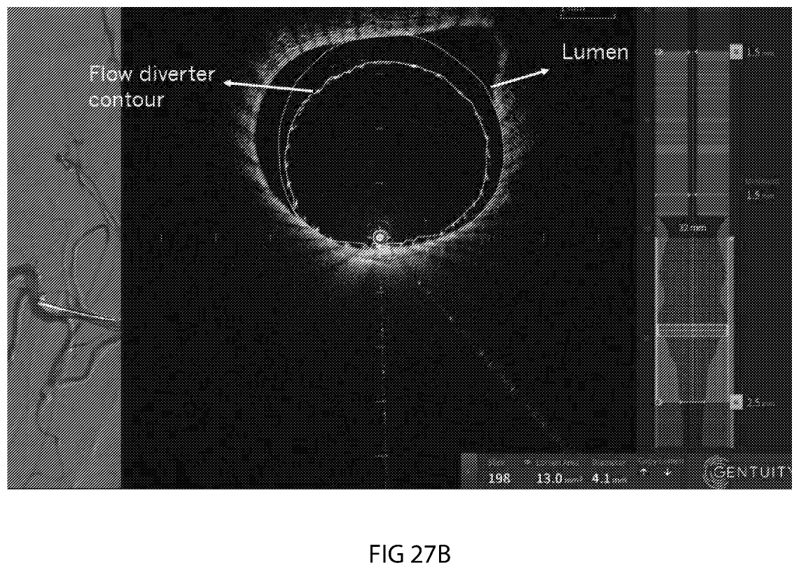

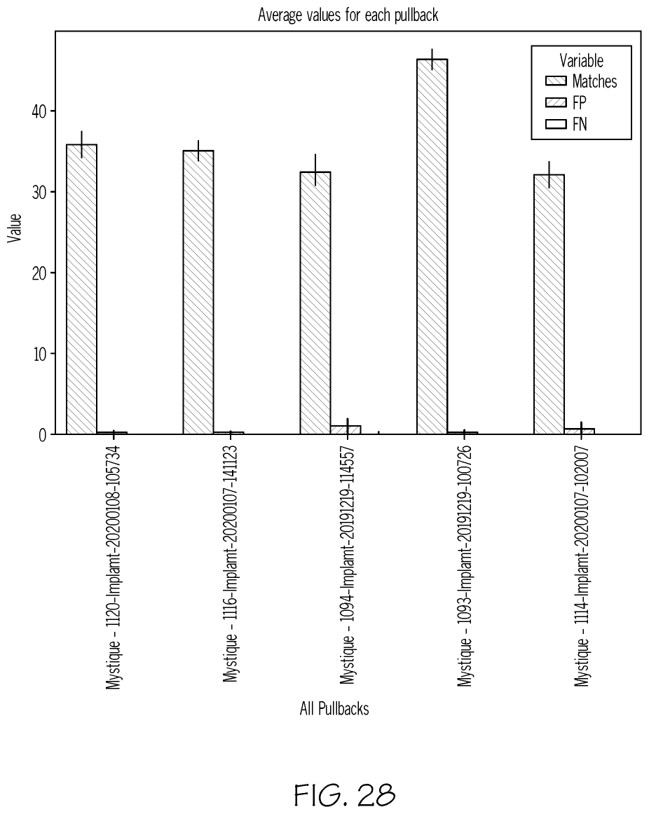

Referring now to FIGS. 27A-28, various representations of data collected by the applicant are illustrated, consistent with the present inventive concepts. In some embodiments, system 10 comprises an AI algorithm, such as algorithm 1015 described herein. Algorithm 1015 can be trained to perform flow diverter detection, such as to automatically identify the coverage by a flow diverter of an aneurysm and/or to identify malposition. Applicant has trained and tested such an algorithm, with training data comprising image data collected from 5 pullbacks, including approximately 3,500 images. Examples of segmented images are shown in FIGS. 27A and 27B. Applicant evaluated the diverter segmentation algorithm by calculating the percentage of actual diverter struts that were segmented relative to the total number of diverter struts in each image frame (e.g. as determined manually and/or by other methods). Testing resulted in an average score of greater than 97.1% across 5 pullbacks. FIG. 28 shows the average matches identified in each pullback along with false positives and false negatives. Testing showed a false positive rate of 0.9% and a false negative rate of 0.05%.

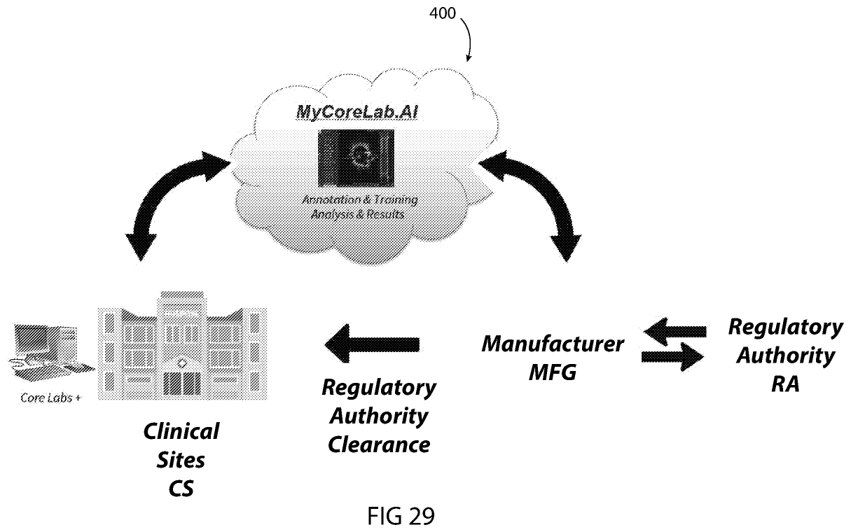

Referring now to FIG. 29, a method of capturing image data, applying AI algorithms on the data to develop improved medical procedures, and obtaining regulatory authority clearance of these procedures is illustrated, consistent with the present inventive concepts. Diagnostic and/or therapeutic medical procedure data, including OCT image data and other clinical data collected by system 10 and other medical devices, is collected at one or more (e.g. many) clinical sites (CS). This collected medical procedure data, “MP data” herein, can be transferred to a centralized data storage and/or processing location, such as server 400 shown and described herein (e.g. a cloud-based server as shown in FIG. 29). MP Data can be transferred from server 400 to one or more clinical sites CS. MP Data (e.g. from multiple patients treated at multiple clinical sites CS) can be analyzed via one or more algorithms of system 10 (e.g. an AI algorithm, such as algorithm 1015 as described herein), such as to generate improved treatment plans for the diagnosis and/or therapeutic treatment of future patients. Output of these AI algorithms can be prepared by the manufacturer MFG of system 10 into one or more regulatory submissions to be submitted to one or more regulatory authorities RA, as shown in FIG. 29. Once a regulatory clearance is achieved, these treatment plans generated by one or more AI algorithms of system 10 can be provided to the clinical sites CS for use in future medical procedures. For example, a regulatory cleared AI algorithm 1015 can comprise an algorithm that analyzes collected MP data (e.g. at least OCT data) and near-immediately provides feedback to the clinician comprising a diagnosis, treatment plan, and/or other medical information for the clinician. In other words, at the time at least some of the analyzed data was collected (e.g. the most recent data was collected), this feedback to the clinician can be provided by system 10, avoiding the need for the MP data to be transferred to an offsite location, analyzed, and returned to the clinical site CS (e.g. avoiding a delay of hours or days).

In some embodiments, MP Data is encrypted before being transferred between server 400 and a clinical site CS, such as to protect patient confidentiality. In some embodiments, each clinical site CS can be assigned a unique private encryption key, such as to prevent (or at least impede) a first site CS from receiving MP data (e.g. accidentally or nefariously) from a second site CS and being able to decrypt the data (e.g. without the unique key). In some embodiments, system 10 encrypts MP data sufficiently to comply with patient privacy laws, for example HIPAA laws.

MP Data captured by system 10 and processed via one or more AI algorithms 1015 of system 10 can include OCT data (e.g. HF-OCT data), angiography data, FFR data, and/or flow data, such as data collected in a pre-treatment procedure, a treatment procedure, and/or a post-treatment follow up procedure. In some embodiments, algorithm 1015 comprises an AI algorithm configured to analyze image data to identify and/or otherwise characterize one or more of: a vessel lumen (e.g. the luminal wall); one or more side-branches; one or more inserted devices (e.g. guide catheter, imaging catheter and/or guidewire); and combinations of these. In some embodiments, algorithm 1015 comprises an AI algorithm configured to analyze image data to identify and/or otherwise characterize one or more of: stenosis (e.g. left main stenosis); diffuse disease; an aneurysm; and combinations of these.

In some embodiments, system 10 can be configured (e.g. via an AI-based algorithm 1015) as a “virtual clinical specialist” or “remote clinical specialist”. For example, system 10 can be configured to perform a procedure assessment, such as a procedure assessment comprising analysis of OCT image data, angiography image data, or both. In these embodiments, system 10 can be configured to assess one or more of: length of pullback; efficacy of a flush procedure; guide catheter engagement; the clearing of blood distal to a lesion; and combinations of these. In these embodiments, system 10 can be configured to provide “real-time coaching” to one or more users of system 10. System 10 can be configured (e.g. via an AI-based algorithm 1015) to provide enhanced image interpretation, such as to redirect clinician time to distinctly human tasks (e.g. interpersonal decision making and/or creative tasks).

In some embodiments, system 10 can be configured (e.g. via an AI-based algorithm 1015) as a “virtual service technician”. For example, system 10 can be configured to analyze (e.g. automatically analyze) image brightness. System 10 can be configured to identify trends across catheters used in one or more clinical procedures. System 10 can be configured to detect an issue with a system 10 component.

In some embodiments, system 10 can be configured (e.g. via an AI-based algorithm 1015) to enhance user (e.g. clinician) performance and/or otherwise improve medical procedure outcomes, such as by: enhancing clinician image interpretation capability; reducing variation between clinical practices; improving procedural success for infrequent users of system 10; and/or minimizing errors.

In some embodiments, system 10 can be configured (e.g. via an AI-based algorithm 1015) to provide predictive information, such as when algorithm 1015 provides predictive indexes of information such as: stent implantation index data; flow diverter implantation index data; coil implantation index data; and combinations of these.

In some embodiments, MP data stored on server 400 (e.g. anonymized MP data) can be accessed by third parties, such as clinical sites CS, and other research collaborators of manufacturer MFG. In some embodiments, a financial transaction is associated with the access to the data and/or receipt of an analysis of the data (e.g. as performed by an AI-based algorithm 1015), such as when the financial transaction comprises a payment made to the manufacturer of system 10. The MP data stored on server 400 can provide a large data moat for the manufacturer of system 10.

In some embodiments, system 10 is configured to encode image data with information related to the processing of the image data. For example, system 10 can include a standard imaging probe 100, and an enhanced imaging probe 100, such as an enhanced probe that encodes image data collected with information enabling advanced image processing. The embedded information can enable or disable analysis features of system 10 based on the imaging probe 100 that was used to collect the image data. In some embodiments, system 10 identifies the type of imaging probe 100 being used by an RFID tag incorporated in the probe.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1. An imaging system for a patient comprising:
an imaging probe, comprising:
an elongate shaft comprising a proximal end, a distal portion, and a lumen extending between the proximal end and the distal portion;
a rotatable optical core comprising a proximal end and a distal end, wherein at least a portion of the rotatable optical core is positioned within the lumen of the elongate shaft; and
an optical assembly positioned proximate the distal end of the rotatable optical core, the optical assembly configured to direct light to tissue to be imaged and to collect reflected light from the tissue to be imaged;
an imaging assembly constructed and arranged to optically couple to the imaging probe, the imaging assembly configured to emit light into the imaging probe and to receive the reflected light collected by the optical assembly; and
a processing unit comprising a processor and a memory coupled to the processor, the memory configured to store instructions for the processor to perform an algorithm;
wherein the system is configured to record image data based on the reflected light collected by the optical assembly,
wherein the image data comprises data collected from a segment of a blood vessel during a pullback procedure,
wherein the algorithm comprises an artificial intelligence algorithm,
wherein the artificial intelligence algorithm is trained to perform a side-branch segmentation, and wherein the algorithm achieves an average Weighted Dice Score of at least 0.81; and
wherein the algorithm is configured to analyze the image data.

2. The system of claim 1, wherein the image data comprises OCT image data.

3. The system of claim 1, wherein the algorithm is configured to calculate computational fluid dynamics of the segment of the blood vessel.

4. The system of claim 1, wherein the algorithm is configured to segment the image data.

5. The system of claim 4, wherein the segmentation is selected from the group consisting of: procedural device segmentation; guide catheter segmentation;
guidewire segmentation; implant segmentation; endovascular implant segmentation;
flow-diverter segmentation; lumen segmentation; side-branch segmentation; and
combinations thereof.

6. The system of claim 4, wherein the algorithm comprises a neural network trained to perform the segmentation.

7. The system of claim 1, wherein the algorithm is configured to produce a confidence metric configured to represent a quality of results of an image processing step.

8. The system of claim 1, wherein the artificial intelligence algorithm comprises at least one a machine learning algorithm, a deep learning algorithm, or a neural network.

9. The system of claim 1, wherein the algorithm comprises a neural network and is configured to skip one or more layers of the neural network.

10. The system of claim 1, wherein the algorithm comprises a single neural network trained to perform two or more image segmentation processes.

11. The system of claim 1, wherein the algorithm is configured to receive image data in a single image domain, and wherein the algorithm is further configured to convert the image data into one or more additional image domains.

12. The system of claim 1, wherein the algorithm is configured to process the image data in one or more image domains selected from the group consisting of: the polar domain; the cartesian domain; the longitudinal domain; the en-face image domain; a domain generated by calculating image features, such as first and/or second order features, image texture, image entropy, homogeneity, correlation, contrast, energy, and/or any other image feature; and combinations thereof.

13. The system of claim 1, further comprising a graphical user interface configured to be displayed to a user.

14. The system of claim 13, wherein the graphical user interface is configured to provide an image data quality indicator.

15. The system of claim 14, wherein the image data quality indicator is displayed relative to a cross-sectional OCT image.

16. The system of claim 13, wherein the graphical user interface is configured to enable a user to review results of an image processing step.

17. The system of claim 16, wherein the graphical user interface is further configured to enable a user to approve the results of the image processing step.

18. The system of claim 16, wherein the graphical user interface is further configured to enable a user to edit the results of the image processing step.

19. The system of claim 16, wherein the algorithm comprises an artificial intelligence algorithm, and wherein the image processing step is performed by the artificial intelligence algorithm.

20. The system of claim 13, wherein the graphical user interface comprises multiple workspaces, and wherein the data displayed in each workspace is synchronized.

21. The system of claim 20, wherein the data is synchronized by a time index.

22. The system of claim 20, wherein the data is synchronized by a location index.

23. The system of claim 1, wherein the system is configured to collect image data prior to an interventional procedure and after the interventional procedure.

24. The system of claim 23, wherein the algorithm is configured to compare a pre-intervention image data and a post-intervention image data and to quantify an effect of the interventional procedure.

25. The system of claim 1, wherein the algorithm comprises a bias.

26. The system of claim 25, further comprising a user interface, wherein the bias can be entered and/or modified via the user interface.
